(19) United States
(12) Patent Application Publication
(10) Pub. No.: US 2026/0007173 A1
(43) Pub. Date: Jan. 8, 2026

(54) SMOKING SUBSTITUTE SYSTEM

(71) Applicant: Imperial Tobacco Limited, Bristol (GB)

(72) Inventors: Kate Ferrie, Liverpool (GB); Ross Shenton, Liverpool (GB); Pete Lomas, Liverpool (GB); Chris Lord, Liverpool (GB); Samantha Murray, Liverpool (GB); Tom Sudlow, Liverpool (GB)

(21) Appl. No.: 19/184,969

(22) Filed: Apr. 21, 2025

Related U.S. Application Data
(63) Continuation of application No. 17/481,889, filed on Sep. 22, 2021, now Pat. No. 12,329,201.
Application No. 17/481,889 is a continuation of application No. PCT/EP2020/056769, filed on Mar. 13, 2020.
Application No. PCT/EP2020/056769 is a continuation of application No. PCT/EP2020/056772, filed on Mar. 13, 2020.
Application No. PCT/EP2020/056772 is a continuation of application No. PCT/EP2020/056776, filed on Mar. 13, 2020.
Application No. PCT/EP2020/056776 is a continuation of application No. PCT/EP2020/056777, filed on Mar. 13, 2020.
Application No. PCT/EP2020/056777 is a continuation of application No. PCT/EP2020/056782, filed on Mar. 13, 2020.
Application No. PCT/EP2020/056782 is a continuation-in-part of application No. PCT/EP2020/056784, filed on Mar. 13, 2020.
Application No. PCT/EP2020/056784 is a continuation of application No. PCT/EP2020/056786, filed on Mar. 13, 2020.
Application No. PCT/EP2020/056786 is a continuation of application No. PCT/EP2020/056788, filed on Mar. 13, 2020.
Application No. PCT/EP2020/056788 is a continuation of application No. PCT/EP2020/056792, filed on Mar. 13, 2020.
Application No. PCT/EP2020/056792 is a continuation of application No. PCT/EP2020/056818, filed on Mar. 13, 2020.
Application No. PCT/EP2020/056818 is a continuation of application No. PCT/EP2020/056822, filed on Mar. 13, 2020.
Application No. PCT/EP2020/056822 is a continuation of application No. PCT/EP2020/056823, filed on Mar. 13, 2020.
Application No. PCT/EP2020/056823 is a continuation of application No. PCT/EP2020/056825, filed on Mar. 13, 2020.
Application No. PCT/EP2020/056825 is a continuation of application No. PCT/EP2020/056836, filed on Mar. 13, 2020.
Application No. PCT/EP2020/056836 is a continuation of application No. PCT/EP2020/056837, filed on Mar. 13, 2020.
Application No. PCT/EP2020/056837 is a continuation of application No. PCT/EP2020/056838, filed on Mar. 13, 2020.
Application No. PCT/EP2020/056838 is a continuation of application No. PCT/EP2020/056854, filed on Mar. 13, 2020.
Application No. PCT/EP2020/056854 is a continuation of application No. PCT/EP2020/056861, filed on Mar. 13, 2020.
Application No. PCT/EP2020/056861 is a continuation of application No. PCT/EP2020/056863, filed on Mar. 13, 2020.
Application No. PCT/EP2020/056863 is a continuation of application No. PCT/EP2020/056868, filed on Mar. 13, 2020.
Application No. PCT/EP2020/056868 is a continuation of application No. PCT/EP2020/056870, filed on Mar. 13, 2020.

(30) Foreign Application Priority Data
Mar. 22, 2019 (EP) 19020137.6
Mar. 22, 2019 (EP) 19020138.4
Mar. 22, 2019 (EP) 19020142.6
Mar. 22, 2019 (EP) 19020147.5
Mar. 22, 2019 (EP) 19020150.9
Mar. 22, 2019 (EP) 19020153.3
Mar. 22, 2019 (EP) 19020155.8
Mar. 22, 2019 (EP) 19020156.6
Mar. 22, 2019 (EP) 19020158.2
Mar. 22, 2019 (EP) 19020159.0
Mar. 22, 2019 (EP) 19020164.0
Mar. 22, 2019 (EP) 19020168.1
Mar. 22, 2019 (EP) 19020169.9
Mar. 22, 2019 (EP) 19020173.1
Mar. 22, 2019 (EP) 19020176.4
Mar. 22, 2019 (EP) 19020179.8
Mar. 22, 2019 (EP) 19020183.0
Mar. 22, 2019 (EP) 19020185.5
Mar. 22, 2019 (EP) 19020189.7
Mar. 22, 2019 (EP) 19020197.0
Mar. 22, 2019 (EP) 19020201.0
Mar. 22, 2019 (EP) 19020203.6
Mar. 22, 2019 (EP) 19020206.9
Mar. 22, 2019 (EP) 19020209.3
Mar. 22, 2019 (EP) 19020210.1
Mar. 22, 2019 (EP) 19020212.7
Mar. 22, 2019 (EP) 19020213.5
Mar. 22, 2019 (EP) 19020216.8
Mar. 22, 2019 (EP) 19020223.4
Feb. 14, 2020 (EP) 20157500.8

(51) Int. Cl. A24F 40/46; A24F 40/20; A24F 40/70; A46B 15/00; A61M 11/04; B25B 27/14; H05B 3/06

(52) U.S. Cl. CPC A24F 40/46; A24F 40/20; A24F 40/70; A46B 15/0055; B25B 27/14; H05B 3/06; A46B 2200/3073; A61M 11/042; A61M 2209/10

(57) ABSTRACT

A smoking substitute device having a body, a heating element extending from the body and a cap removably attached to the body. The cap is movable between a first position and a second position along a longitudinal axis of the body. In the first position the heating element is concealed in the cap and in the second position the heating element is at least partially exposed.

### CROSS REFERENCE TO RELATED APPLICATIONS/INCORPORATION BY REFERENCE STATEMENT

This application is a continuation of U.S. nonprovisional utility patent application Ser. No. 17/481,889 filed on 22 Sep. 2021, which is a United States nonprovisional utility patent application claiming benefit to all of the following international patent applications: international application no. PCT/EP2020/056769 filed on 13 Mar. 2020, which claims priority to EP 19020153.3 filed on 22 Mar. 2019 and to EP 20157500.8 filed on 14 Feb. 2020; international application no. PCT/EP2020/056772 filed on 13 Mar. 2020, which claims priority to EP 19020150.9 filed on 22 Mar. 2019; international application no. PCT/EP2020/056776 filed on 13 Mar. 2020, which claims priority to EP 19020137.6 filed on 22 Mar. 2019, EP 19020138.4 filed on 22 Mar. 2019, EP 19020159.0 filed on 22 Mar. 2019, EP 19020173.1 filed on 22 Mar. 2019, EP 19020176.4 filed on 22 Mar. 2019, EP 19020185.5 filed on 22 Mar. 2019, EP 19020189.7 filed on 22 Mar. 2019, EP 19020210.1 filed on 22 Mar. 2019, EP 19020213.5 filed on 22 Mar. 2019, and EP 19020169.9 filed on 22 Mar. 2019; international application no. PCT/EP2020/056777 filed on 13 Mar. 2020, which claims priority to EP 19020183.0 filed on 22 Mar. 2019; international application no. PCT/EP2020/056782 filed on 13 Mar. 2020, which claims priority to EP 19020179.8 filed on 22 Mar. 2019; international application no. PCT/EP2020/056784 filed on 13 Mar. 2020, which claims priority to EP 19020216.8 filed on 22 Mar. 2019; international application no. PCT/EP2020/056786 filed on 13 Mar. 2020, which claims priority to EP 19020212.7 filed on 22 Mar. 2019; international application no. PCT/EP2020/056788 filed on 13 Mar. 2020, which claims priority to EP 19020209.3 filed on 22 Mar. 2019; international application no. PCT/EP2020/056792 filed on 13 Mar. 2020, which claims priority to EP 19020203.6 filed on 22 Mar. 2019; international application no. PCT/EP2020/056818 filed on 13 Mar. 2020, which claims priority to EP 19020168.1 filed on 22 Mar. 2019; international application no. PCT/EP2020/056822 filed on 13 Mar. 2020, which claims priority to EP 19020155.8 filed on 22 Mar. 2019; international application no. PCT/EP2020/056823 filed on 13 Mar. 2020, which claims priority to EP 19020156.6 filed on 22 Mar. 2019; international application no. PCT/EP2020/056825 filed on 13 Mar. 2020, which claims priority to EP 19020159.0 filed on 22 Mar. 2019; international application no. PCT/EP2020/056836 filed on 13 Mar. 2020, which claims priority to EP 19020164.0 filed on 22 Mar. 2019; international application no. PCT/EP2020/056837 filed on 13 Mar. 2020, which claims priority to EP 19020223.4 filed on 22 Mar. 2019; international application no. PCT/EP2020/056838 filed on 13 Mar. 2020, which claims priority to EP 19020158.2 filed on 22 Mar. 2019; international application no. PCT/EP2020/056854 filed on 13 Mar. 2020, which claims priority to EP 19020147.5 filed on 22 Mar. 2019; international application no. PCT/EP2020/056861 filed on 13 Mar. 2020, which claims priority to EP 19020197.0 filed on 22 Mar. 2019; international application no. PCT/EP2020/056863 filed on 13 Mar. 2020, which claims priority to EP 19020142.6 filed on 22 Mar. 2019; international application no. PCT/EP2020/056868 filed on 13 Mar. 2020, which claims priority to EP 19020201.0 filed on 22 Mar. 2019; and international application no. PCT/EP2020/056870 filed on 13 Mar. 2020, which claims priority to EP 19020206.9 filed on 22 Mar. 2019. The entire contents of each of the above referenced applications are hereby incorporated herein by reference in their entirety.

### FIELD OF THE DISCLOSURE

The present invention relates to a smoking substitute system and particularly, although not exclusively, to a smoking substitute system comprising a smoking substitute device and a tool for the device.

The present disclosure also relates to a smoking substitute system and particularly, although not exclusively, to a smoking substitute system comprising a smoking substitute device and an aerosol-forming article.

### BACKGROUND

The smoking of tobacco is generally considered to expose a smoker to potentially harmful substances. It is generally thought that a significant amount of the potentially harmful substances is generated through the heat caused by the burning and/or combustion of the tobacco and the constituents of the burnt tobacco in the tobacco smoke itself.

Conventional combustible smoking articles, such as cigarettes, typically comprise a cylindrical rod of tobacco comprising shreds of tobacco which is surrounded by a wrapper, and usually also a cylindrical filter axially aligned in an abutting relationship with the wrapped tobacco rod. The filter typically comprises a filtration material which is circumscribed by a plug wrap. The wrapped tobacco rod and the filter are joined together by a wrapped band of tipping paper that circumscribes the entire length of the filter and an adjacent portion of the wrapped tobacco rod. A conventional cigarette of this type is used by lighting the end opposite to the filter, and burning the tobacco rod. The smoker receives mainstream smoke into their mouth by drawing on the mouth end or filter end of the cigarette.

Combustion of organic material such as tobacco is known to produce tar and other potentially harmful by-products. There have been proposed various smoking substitute systems (or “substitute smoking systems”) in order to avoid the smoking of tobacco.

Such smoking substitute systems can form part of nicotine replacement therapies aimed at people who wish to stop smoking and overcome a dependence on nicotine.

Smoking substitute systems include electronic systems that permit a user to simulate the act of smoking by producing an aerosol (also referred to as a “vapor”) that is drawn into the lungs through the mouth (inhaled) and then exhaled. The inhaled aerosol typically bears nicotine and/or flavorings without, or with fewer of, the odor and health risks associated with traditional smoking.

In general, smoking substitute systems are intended to provide a substitute for the rituals of smoking, whilst providing the user with a similar experience and satisfaction to those experienced with traditional smoking and with combustible tobacco products. Some smoking substitute systems use smoking substitute articles (also referred to as a “consumable”) that are designed to resemble a traditional cigarette and are cylindrical in form with a mouthpiece at one end.

The popularity and use of smoking substitute systems has grown rapidly in the past few years. Although originally marketed as an aid to assist habitual smokers wishing to quit tobacco smoking, consumers are increasingly viewing smoking substitute systems as desirable lifestyle accessories.

There are a number of different categories of smoking substitute systems, each utilizing a different smoking substitute approach.

One approach for a smoking substitute system is the so-called Heated Tobacco (“HT”) approach in which tobacco (rather than an “e-liquid”) is heated or warmed to release vapor. HT is also known as “heat not burn” (“HNB”). The tobacco may be leaf tobacco or reconstituted tobacco. The vapor may contain nicotine and/or flavorings. In the HT approach the intention is that the tobacco is heated but not burned, i.e., the tobacco does not undergo combustion.

A typical HT smoking substitute system may include a device and a consumable. The consumable may include the tobacco material. The device and consumable may be configured to be physically coupled together. For example, the consumable may be inserted into a cavity or heating chamber and thereby establishing physical contact with a heating element located in the cavity. In use, heat may be imparted to the tobacco material by a heating element of the device, wherein airflow through the tobacco material causes components in the tobacco material to be released as vapor. A vapor may also be formed from a carrier in the tobacco material (this carrier may for example include propylene glycol and/or vegetable glycerin) and additionally volatile compounds released from the tobacco. The released vapor may be entrained in the airflow drawn through the tobacco.

As the vapor passes through the consumable (entrained in the airflow) from the location of vaporization to an outlet of the consumable (e.g., a mouthpiece), the vapor cools and condenses to form an aerosol for inhalation by the user. The aerosol will normally contain the volatile compounds.

In HT smoking substitute systems, heating as opposed to burning the tobacco material is believed to cause fewer, or smaller quantities, of the more harmful compounds ordinarily produced during smoking. Consequently, the HT approach may reduce the odor and/or health risks that can arise through the burning, combustion and pyrolytic degradation of tobacco.

During use, physical contact between the heating element and the tobacco material causes residue to form on the surface of the heating element. Therefore, HT devices often require frequent cleaning with a specialized tool to avoid residue built up on the heater surface. For example, the user may require to clean the heating element with the use of a brush or a disposable solvent swap. However, currently available HT systems may only provide access to the heating element through an opening towards at the end of the cavity. Further, the user may require removing a cap covering said opening to gain access to the heating element prior to cleaning. Such arrangement may be inconvenient.

Therefore, some users may only clean the heating element once the device has consumed a given number of consumables, e.g., twenty (20) consumables, when the residue built up becomes detrimental to the experience, e.g., when a burnt taste is perceivable.

It is often the case that residual debris left by a consumable remains within the body of a HNB device after use. For example, pieces of tobacco may become dislodged from the consumable during use, falling into the cavity of the device in which the consumable resides. In some cases, the debris may remain in contact with the heater inside the device, which is a safety risk since the debris could eventually burn or ignite. This could also impair the flavor of a subsequent consumable. It is often difficult for a user to properly clean the heating element between smoking cycles to ensure that such debris is removed.

There may be a need for improved design of smoking substitute devices, in particular HT smoking substitute devices, to enhance the user experience and improve the function of the HT smoking substitute system.

The present disclosure has been devised in the light of the above considerations.

### SUMMARY OF THE DISCLOSURE

First Mode: A Smoking Substitute System with a Smoking Substitute Device Having a Cap Movable Between Two Positions

At its most general, a first mode of the present invention relates to a smoking substitute system with a smoking substitute device having a cap movable between two positions to selectively conceal or expose a heating element of the smoking substitute device. This may allow the user to physically access and clean the heating element in a more convenient manner, and thereby facilitate a more frequent cleaning routine. The present invention also relates to a tool for removing the cap form the device, and thereby preventing inadvertent removal of the cap. The tool may further comprise a cleaning means to conveniently allow the user to clean the heating element once the cap is removed by the tool.

According to a first aspect of the first mode of the present invention, there is provided a smoking substitute device having a body, a heating element extending from the body and a cap removably attached to the body. The cap is movable between a first position and a second position along a longitudinal axis of the body. In the first position the heating element is concealed in the cap and in the second position the heating element is at least partially exposed.

For example, in the first position, the cap may cover a window or an opening at the sidewall of the body that extends into a transverse cavity containing the heating element, and thereby conceals the heating element. In the second position, the cap is moved or slide to a location where it may no longer cover the opening, and thereby the heating element may be at least partially exposed through the opening. More specifically, the opening may be located adjacent to exposed portion of the heating element and therefore it may provide physical access to said exposed portion of the heating element.

By providing a device comprising a cap movable between two positions, the heating element may be cleaned in a more convenient manner. For example, when the cap is moved to the second position, the heating element may be exposed through a window or opening from the side of the device, as such said heating element may be visually inspected or cleaned through said opening. Advantageously, the user may thereby carry out a brief cleaning at the heating element without requiring a dedicated cleaning tool. For example, the user may simply blow through the opening or physically shaking, tilting and/or tapping the device to dislodge loose debris that are formed on the heating element. The user may also physically clean the exposed portion of the heating element, e.g., a base of the heating element, with the use of a tool, e.g., a brush. Further, the smoking substitute as disclosed herein may prolong the usability of the device before it requires deep cleaning or other such maintenance.

Optional features will now be set out. These are applicable singly or in any combination with any aspect of the first mode.

Optionally, the cap may be retainable on the body in the second position by a retaining means. Optionally, the retaining means may be any suitable retaining means, for example interference fit or latch mechanism. Advantageously, said retaining means may allow the cap to be positioned and retained in the second position during visual inspection and/or cleaning, and may stop it from moving further along the longitudinal axis once it reaches the second position, thereby it may prevent the cap from being inadvertently removed from the body.

Optionally, the retaining means comprises at least one flexible locking arm extending from the body, and a locking protrusion disposed on the at least one locking arm. The locking protrusion may be configured to engage a slot defined in the cap to retain the cap on the body. Optionally, the locking protrusion may be retained in the slot when the cap moves between the first position and the second position. Advantageously, the locking protrusion may slide along the slot when the cap moves from the first position to the second position, and thereby prevents relative rotation between the cap and the body.

Optionally, the body defines a transverse cavity that opens through a first side wall of the body, the opening may be juxtaposed with a base of the heating element to at least partially expose the base of the heating element when the cap is in the second position. For example, the opening at the side wall of body leads to said transverse cavity. The transverse cavity may be juxtaposed lateral to the base of the heating element, or in other words, the transverse cavity may open in a direction orthogonal to the longitudinal axis of the body.

Optionally, the transverse cavity may extend from the first side wall of the body to a second side wall opposite to the first side wall. In other words, the transverse cavity may be a through hole extending through both the first side wall and the second side wall of the body. Advantageously, this may allow loose debris to be effectively discharged from the opening or through hole.

Optionally, the locking protrusion is configured to prevent separation of the cap from the device by abutting an end of the slot once the cap has moved to the second position, as such blocking further movement of the cap. In other words, the locking protrusion may prevent detachment of the cap by blocking movement of the cap by abutting a peripheral surface of the cap defining the slot when the cap is in the second position.

Optionally, the cap may define a cavity for receiving at least a portion of an aerosol-forming article. Optionally, the slot may be connected with or open to the cavity.

In a second aspect of the first mode according to the present invention, there is provided a tool for separating the cap from the body of the smoking substitute device is disclosed. The tool may be used to separate or dislodge the cap from the device for a deep cleaning of the heating element. The tool has unlocking means at one end and a cleaning means at another end. The tool may provide for a compact and easy maintenance of the smoking substitute device of the smoking substitute system of the present disclosure.

The tool may have at least one unlocking arm and an unlocking protrusion disposed on the unlocking arm. The unlocking protrusion may be configured to displace a corresponding locking protrusion disposed on a locking arm extending from the body of the device to disengage the locking protrusion from a slot in the cap. Advantageously, the tool prevents the inadvertent removal of the cap from the body of the device.

Optionally, the tool further comprises a cleaning means for cleaning the heating element. Advantageously, the cleaning means to conveniently allow the user to physically clean the heating element once the cap is removed by the tool, and thereby allowing the heating element to be better clean, e.g., to “deep clean” the heating element. Optionally, the cleaning means comprises at least one cleaning bristle. Advantageously, in use the bristle may scrape on the surface of the heating element, and thereby it may allow the heating element to be cleaned in a more efficient manner.

Optionally, the tool further comprises a central rod, the at least one unlocking arm extending along a longitudinal axis of the central rod in a first direction and the cleaning means extending in a second direction opposite to the first direction.

Optionally, the tool comprises a collar around the central rod having the unlocking arm extended in the first direction, the collar being movable between an insertion position and an unlocking position, wherein in the insertion position the at least one unlocking arm is allowed to flex and in the unlocking position the central rod prevents the flexing of the unlocking arm. The collar may comprise a ring-shaped collar. The collar may be positioned concentrically on the central rod.

Optionally, the tool further comprises a first cover configured to cover the at least one unlocking arm and a second cover configured to cover the cleaning means.

Optionally, the tool having an external profile similar to that of an aerosol-forming article for a smoking substitute system.

The device may comprise an elongate body. An end of the elongate body may be configured for engagement with an aerosol-forming article. For example, the body may be configured for engagement with a heated tobacco (HT) consumable (or heat-not-burn (HNB) consumable). The terms “heated tobacco” and “heat-not-burn” are used interchangeably herein to describe a consumable that is of the type that is heated rather than combusted (or are used interchangeably to describe a device for use with such a consumable). The device may comprise a cavity that is configured for receipt of at least a portion of the consumable (i.e., for engagement with the consumable). The aerosol-forming article may be of the type that comprises an aerosol former (e.g., carried by an aerosol-forming substrate).

The body may define a transverse cavity extending orthogonal to the longitudinal axis of the body. The transverse cavity may extend laterally and may be located on the body such that at least the base of the heating element is juxtaposed with the transverse cavity.

Further, at least one locking arm may extend from the body. The locking arms may lock or retain the cap with the body. The locking arms may be provided with a locking protrusion at a distal end. The locking protrusion may extend transversely to the longitudinal axis of the body. The locking arms may be positioned such that when the cap is mounted on the body, the locking arms may engage the cap to retain the cap on the body.

The cap may be provided with a slot extending along the longitudinal axis of the body (when the cap is retained on the body), and the locking protrusions may be configured to engage the slot. The slot may be elongated such that the cap may be moved or slide relative to the body along the longitudinal axis of the body. The locking protrusion may have an abutment surface to engage a peripheral surface of the cap that defines the slot to retain or lock the cap with the body.

The cap may be movable between a first position and a second position. When the cap is in the first position, the cap may conceal the heating element. When the cap is in the second position, the heating element may be at least partially exposed, e.g., through a window or opening at the side wall of the body. When the heating element is partially exposed, the heating element may be examined visually to ascertain if cleaning of the heating element is required. If required, when the cap is in the second position, the heating element may be at least partly cleaned by blowing air through the opening or simply shaking, tilting and or tapping the device gently to dislodge and remove the debris.

The smoking substitute system of the present disclosure may further include a tool for separation of the cap from the body. The tool may be configured to displace the locking arms to enable separation of the cap from the body. The tool may comprise at least one unlocking arm. The unlocking arms may be adapted to engage the locking arms to displace the locking arms for separating the cap from the body. Each unlocking arm may be provided with an unlocking protrusion. The unlocking protrusion may extend in a direction orthogonal to the longitudinal axis of the unlocking arm. The unlocking protrusions may be adapted to engage the locking protrusions to displace the locking protrusions for releasing the cap from the body.

The tool may further include a central rod. A collar may be positioned concentrically on the central rod. The collar may be placed movably on the rod such that the collar moves relative to the central rod along a longitudinal axis of the central rod. The unlocking arms may extend from the collar along the longitudinal axis of the central rod. The collar may be movable on the central rod between an insertion portion and an unlocking position. In the insertion position, the central rod may be kept away from the unlocking protrusions and the unlocking arms may flex radially inwards relative to the longitudinal axis of the central rod. In the unlocking position, the central rod may move adjacent to the unlocking protrusions to prevent flexing of the unlocking arms in a direction radially inwards relative to the longitudinal axis of the central rod. The collar may be biased to move towards the insertion position using any suitable means such as a coil spring.

The tool may be configured for insertion into the cavity. The unlocking protrusions may be configured such that when the unlocking arms are inserted into the cavity, the unlocking arm displaces the locking arms to release engagement of the locking arms from the slots. The unlocking protrusions may have dimensions that interfere with the width of the cavity. In order to allow insertion of the unlocking arms in the cavity, in the insertion position, the central rod may be spaced from the distal ends of the unlocking arms to allow the distal ends of the unlocking arms to flex radially inwards to enable insertion of the unlocking arms with the unlocking protrusions into the cavity. The flexing may be achieved when the unlocking protrusions abut and slide against an inner surface of cap defining the internal cavity. The unlocking protrusions, as shown in the embodiment illustrated, may be provided with tapered surfaces to guide the flexing movement of the unlocking arms in and out from the cavity and the slots.

The tool may further comprise a cleaning means for cleaning the heating element. The cleaning means may be in form of cleaning bristles. The cleaning bristles may be rubbed on the outer surface of the heating element to clean or scrap off any debris or residuals from the heating element.

The tool may include a first cover to cover the unlocking arms when not in use. Further, a second cover may be provided to cover the cleaning bristles when not in use.

The device may comprise a heater for heating the aerosol-forming article. The heater may comprise a heating element, which may be in the form of a rod that extends from the body of the device. The heating element may extend from the end of the body that is configured for engagement with the aerosol-forming article.

The heater (and thus the heating element) may be rigidly mounted to the body. The heating element may be elongate so as to define a longitudinal axis and may, for example, have a transverse profile (i.e., transverse to a longitudinal axis of the heating element) that is substantially circular (i.e., the heating element may be generally cylindrical). Alternatively, the heating element may have a transverse profile that is rectangular (i.e., the heater may be a “blade heater”). The heating element may alternatively be in the shape of a tube (i.e., the heater may be a “tube heater”). The heating element may take other forms (e.g., the heating element may have an elliptical transverse profile). The shape and/or size (e.g., diameter) of the transverse profile of the heating element may be generally consistent for the entire length (or substantially the entire length) of the heating element.

The heating element may be between 15 mm and 25 mm long, e.g., between 18 mm and 20 mm long, e.g., around 19 mm long. The heating element may have a diameter of between 1.5 mm and 2.5 mm, e.g., a diameter between 2 mm and 2.3 mm, e.g., a diameter of around 2.15 mm.

The heating element may be formed of ceramic. The heating element may comprise a core (e.g., a ceramic core) comprising Al2O3. The core of the heating element may have a diameter of 1.8 mm to 2.1 mm, e.g., between 1.9 mm and 2 mm. The heating element may comprise an outer layer (e.g., an outer ceramic layer) comprising Al2O3. The thickness of the outer layer may be between 160 μm and 220 μm, e.g., between 170 μm and 190 μm, e.g., around 180 μm. The heating element may comprise a heating track, which may extend longitudinally along the heating element. The heating track may be sandwiched between the outer layer and the core of the heating element. The heating track may comprise tungsten and/or rhenium. The heating track may have a thickness of around 20 μm.

The heating element may be located in the cavity (of the device), and may extend (e.g., along a longitudinal axis) from an internal base of the cavity towards an opening of the cavity. The length of the heating element (i.e., along the longitudinal axis of the heater) may be less than the depth of the cavity. Hence, the heating element may extend for only a portion of the length of the cavity. That is, the heating element may not extend through (or beyond) the opening of the cavity.

The heating element may be configured for insertion into an aerosol-forming article (e.g., a HT consumable) when an aerosol-forming article is received in the cavity. In that respect, a distal end (i.e., distal from a base of the heating element where it is mounted to the device) of the heating element may comprise a tapered portion, which may facilitate insertion of the heating element into the aerosol-forming article. The heating element may fully penetrate an aerosol-forming article when the aerosol-forming article is received in the cavity. That is, the entire length, or substantially the entire length, of the heating element may be received in the aerosol-forming article.

The heating element may have a length that is less than, or substantially the same as, an axial length of an aerosol-forming substrate forming part of an aerosol-forming article (e.g., a HT consumable). Thus, when such an aerosol-forming article is engaged with the device, the heating element may only penetrate the aerosol-forming substrate, rather than other components of the aerosol-forming article. The heating element may penetrate the aerosol-forming substrate for substantially the entire axial length of the aerosol forming-substrate of the aerosol-forming article. Thus, heat may be transferred from (e.g., an outer circumferential surface of) the heating element to the surrounding aerosol-forming substrate, when penetrated by the heating element. That is, heat may be transferred radially outwardly (in the case of a cylindrical heating element) or e.g., radially inwardly (in the case of a tube heater).

Where the heater is a tube heater, the heating element of the tube heater may surround at least a portion of the cavity. When the portion of the aerosol-forming article is received in the cavity, the heating element may surround a portion of the aerosol-forming article (i.e., so as to heat that portion of the aerosol-forming article). In particular, the heating element may surround an aerosol forming substrate of the aerosol-forming article. That is, when an aerosol-forming article is engaged with the device, the aerosol forming substrate of the aerosol-forming article may be located adjacent an inner surface of the (tubular) heating element. When the heating element is activated, heat may be transferred radially inwardly from the inner surface of the heating element to heat the aerosol forming substrate.

The cavity may comprise a (e.g., circumferential) wall (or walls) and the (tubular) heating element may extend around at least a portion of the wall(s). In this way, the wall may be located between the inner surface of the heating element and an outer surface of the aerosol-forming article. The wall (or walls) of the cavity may be formed from a thermally conductive material (e.g., a metal) to allow heat conduction from the heating element to the aerosol-forming article. Thus, heat may be conducted from the heating element, through the cavity wall (or walls), to the aerosol-forming substrate of an aerosol-forming article received in the cavity.

In some embodiments the device may comprise a cap disposed at the end of the body that is configured for engagement with an aerosol-forming article. Where the device comprises a heater having a heating element, the cap may at least partially enclose the heating element. The cap may be moveable between an open position, or a second position, in which access is provided to the heating element, and a closed position in which the cap at least partially encloses the heating element. The cap may be slidably engaged with the body of the device, and may be sliceable between the open and closed positions.

The cap may define at least a portion of the cavity of the device. That is, the cavity may be fully defined by the cap, or each of the cap and body may define a portion of the cavity. Where the cap fully defines the cavity, the cap may comprise an aperture for receipt of the heating element into the cavity (when the cap is in the closed position). The cap may comprise an opening to the cavity. The opening may be configured for receipt of at least a portion of an aerosol-forming article. That is, an aerosol-forming article may be inserted through the opening and into the cavity (so as to be engaged with the device).

The cap may be configured such that when an aerosol-forming article is engaged with the device (e.g., received in the cavity), only a portion of the aerosol-forming article is received in the cavity. That is, a portion of the aerosol-forming article (not received in the cavity) may protrude from (i.e., extend beyond) the opening. This (protruding) portion of the aerosol-forming article may be a terminal (e.g., mouth) end of the aerosol-forming article, which may be received in a user's mouth for the purpose of inhaling aerosol formed by the device.

The device may comprise a power source or may be connectable to a power source (e.g., a power source separate to the device). The power source may be electrically connectable to the heater. In that respect, altering (e.g., toggling) the electrical connection of the power source to the heater may affect a state of the heater. For example, toggling the electrical connection of the power source to the heater may toggle the heater between an on state and an off state. The power source may be a power store. For example, the power source may be a battery or rechargeable battery (e.g., a lithium-ion battery).

The device may comprise an input connection (e.g., a USB port, Micro USB port, USB-C port, etc.). The input connection may be configured for connection to an external source of electrical power, such as a mains electrical supply outlet. The input connection may, in some cases, be used as a substitute for an internal power source (e.g., battery or rechargeable battery). That is, the input connection may be electrically connectable to the heater (for providing power to the heater). Hence, in some forms, the input connection may form at least part of the power source of the device.

Where the power source comprises a rechargeable power source (such as a rechargeable battery), the input connection may be used to charge and recharge the power source.

The device may comprise a user interface (UI). In some embodiments the UI may include input means to receive operative commands from the user. The input means of the UI may allow the user to control at least one aspect of the operation of the device. In some embodiments the input means may comprise a power button to switch the device between an on state and an off state.

In some embodiments the UI may additionally or alternatively comprise output means to convey information to the user. In some embodiments the output means may comprise a light to indicate a condition of the device (and/or the aerosol-forming article) to the user. The condition of the device (and/or aerosol-forming article) indicated to the user may comprise a condition indicative of the operation of the heater. For example, the condition may comprise whether the heater is in an off state or an on state. In some embodiments, the UI unit may comprise at least one of a button, a display, a touchscreen, a switch, a light, and the like. For example, the output means may comprise one or more (e.g., two, three, four, etc.) light-emitting diodes (“LEDs”) that may be located on the body of the device.

The device may further comprise a puff sensor (e.g., airflow sensor), which form part of the input means of the UI. The puff sensor may be configured to detect a user drawing on an end (i.e., a terminal (mouth) end) of the aerosol-forming article. The puff sensor may, for example, be a pressure sensor or a microphone. The puff sensor may be configured to produce a signal indicative of a puff state. The signal may be indicative of the user drawing (an aerosol from the aerosol-forming article) such that it is e.g., in the form of a binary signal. Alternatively, or additionally, the signal may be indicative of a characteristic of the draw (e.g., a flow rate of the draw, length of time of the draw, etc.).

The device may comprise a controller, or may be connectable to a controller that may be configured to control at least one function of the device. The controller may comprise a microcontroller that may e.g., be mounted on a printed circuit board (PCB). The controller may also comprise a memory, e.g., non-volatile memory. The memory may include instructions, which, when implemented, may cause the controller to perform certain tasks or steps of a method. Where the device comprises an input connection, the controller may be connected to the input connection.

The controller may be configured to control the operation of the heater (and e.g., the heating element). Thus, the controller may be configured to control vaporization of an aerosol forming part of an aerosol-forming article engaged with the device. The controller may be configured to control the voltage applied by power source to the heater. For example, the controller may be configured to toggle between applying a full output voltage (of the power source) to the heater and applying no voltage to the heater. Alternatively, or additionally, the control unit may implement a more complex heater control protocol.

The device may further comprise a voltage regulator to regulate the output voltage supplied by the power source to form a regulated voltage. The regulated voltage may subsequently be applied to the heater.

In some embodiments, where the device comprises a UI, the controller may be operatively connected to one or more components of the UI. The controller may be configured to receive command signals from an input means of the UI. The controller may be configured to control the heater in response to the command signals. For example, the controller may be configured to receive “on” and “off” command signals from the UI and, in response, may control the heater so as to be in a corresponding on or off state.

The controller may be configured to send output signals to a component of the UI. The UI may be configured to convey information to a user, via an output means, in response to such output signals (received from the controller). For example, where the device comprises one or more LEDs, the LEDs may be operatively connected to the controller. Hence, the controller may be configured to control the illumination of the LEDs (e.g., in response to an output signal). For example, the controller may be configured to control the illumination of the LEDs according to (e.g., an on or off) state of the heater.

Where the device comprises a sensor (e.g., a puff/airflow sensor), the controller may be operatively connected to the sensor. The controller may be configured to receive a signal from the sensor (e.g., indicative of a condition of the device and/or engaged aerosol-forming article). The controller may be configured to control the heater, or an aspect of the output means, based on the signal from the sensor.

The device may comprise a wireless interface configured to communicate wirelessly (e.g., via Bluetooth (e.g., a Bluetooth low-energy connection) or Wi-Fi) with an external device. Similarly, the input connection may be configured for wired connection to an external device so as to provide communication between the device and the external device. The external device may be a mobile device. For example, the external device may be a smart phone, tablet, smart watch, or smart car. An application (e.g., app) may be installed on the external device (e.g., mobile device). The application may facilitate communication between the device and the external device via the wired or wireless connection.

The wireless or wired interface may be configured to transfer signals between the external device and the controller of the device. In this respect, the controller may control an aspect of the device in response to a signal received from an external device. Alternatively, or additionally, an external device may respond to a signal received from the device (e.g., from the controller of the device).

In a third aspect of the first mode, there is provided a system (e.g., a smoking substitute system) comprising the device according to the first aspect of the first mode and an aerosol-forming article. The aerosol-forming article may comprise an aerosol-forming substrate at an upstream end of the aerosol-forming article. The article may be in the form of a smoking substitute article, e.g., heated tobacco (HT) consumable (also known as a heat-not-burn (HNB) consumable).

As used herein, the terms “upstream” and “downstream” are intended to refer to the flow direction of the vapor/aerosol i.e., with the downstream end of the article/consumable being the mouth end or outlet where the aerosol exits the consumable for inhalation by the user. The upstream end of the article/consumable is the opposing end to the downstream end.

The aerosol-forming substrate is capable of being heated to release at least one volatile compound that can form an aerosol. The aerosol-forming substrate may be located at the upstream end of the article/consumable.

In order to generate an aerosol, the aerosol-forming substrate comprises at least one volatile compound that is intended to be vaporized/aerosolized and that may provide the user with a recreational and/or medicinal effect when inhaled. Suitable chemical and/or physiologically active volatile compounds include the group consisting of: nicotine, cocaine, caffeine, opiates and opioids, cathine and cathinone, kavalactones, mysticin, beta-carboline alkaloids, salvinorin A together with any combinations, functional equivalents to, and/or synthetic alternatives of the foregoing.

The aerosol-forming substrate may comprise plant material. The plant material may comprise least one plant material selected from the list including Amaranthus dubius, Arctostaphylos uva-ursi (Bearberry), Argemone mexicana, Amica, Artemisia vulgaris, Yellow Tees, Galea zacatechichi, Canavalia maritima (Baybean), Cecropia mexicana (Guamura), Cestrum noctumum, Cynoglossum virginianum (wild comfrey), Cytisus scoparius, Damiana, Entada rheedii, Eschscholzia californica (California Poppy), Fittonia albivenis, Hippobroma longiflora, Humulus japonica (Japanese Hops), Humulus lupulus (Hops), Lactuca virosa (Lettuce Opium), Laggera alata, Leonotis leonurus, Leonurus cardiaca (Motherwort), Leonurus sibiricus (Honeyweed), Lobelia cardinalis, Lobelia inflata (Indian-tobacco), Lobelia siphilitica, Nepeta cataria (Catnip), Nicotiana species (Tobacco), Nymphaea alba (White Lily), Nymphaea caerulea (Blue Lily), Opium poppy, Passiflora incamata (Passionflower), Pedicularis densiflora (Indian Warrior), Pedicularis groenlandica (Elephant's Head), Salvia divinorum, Salvia dorrii (Tobacco Sage), Salvia species (Sage), Scutellaria galericulata, Scutellaria lateriflora, Scutellaria nana, Scutellaria species (Skullcap), Sida acuta (Wireweed), Sida rhombifolia, Silene capensis, Syzygium aromaticum (Clove), Tagetes lucida (Mexican Tarragon), Tarchonanthus camphoratus, Tumera diffusa (Damiana), Verbascum (Mullein), Zamia latifolia (Maconha Brava) together with any combinations, functional equivalents to, and/or synthetic alternatives of the foregoing.

The plant material may be tobacco. Any type of tobacco may be used. This includes, but is not limited to, flue-cured tobacco, burley tobacco, Maryland Tobacco, dark-air cured tobacco, oriental tobacco, dark-fired tobacco, perique tobacco and rustica tobacco. This also includes blends of the above-mentioned tobaccos.

The tobacco may comprise one or more of leaf tobacco, stem tobacco, tobacco powder, tobacco dust, tobacco derivatives, expanded tobacco, homogenized tobacco, shredded tobacco, extruded tobacco, cut rag tobacco and/or reconstituted tobacco (e.g., slurry recon or paper recon).

The aerosol-forming substrate may comprise a gathered sheet of homogenized (e.g., paper/slurry recon) tobacco or gathered shreds/strips formed from such a sheet.

The aerosol-forming substrate may comprise one or more additives selected from humectants, flavorants, fillers, aqueous/non-aqueous solvents and binders.

The flavorant may be provided in solid or liquid form. It may include menthol, licorice, chocolate, fruit flavor (including e.g., citrus, cherry etc.), vanilla, spice (e.g., ginger, cinnamon) and tobacco flavor. The flavorant may be evenly dispersed throughout the aerosol-forming substrate or may be provided in isolated locations and/or varying concentrations throughout the aerosol-forming substrate.

The aerosol-forming substrate may be formed in a substantially cylindrical shape such that the article/consumable resembles a conventional cigarette. It may have a diameter of between 5 and 10 mm e.g., between 6 and 9 mm or 6 and 8 mm e.g., around 7 mm. It may have an axial length of between 10 and 15 mm e.g., between 11 and 14 mm such as around 12 or 13 mm.

The article/consumable may comprise at least one filter element. There may be a terminal filter element at the downstream/mouth end of the article/consumable.

The or at least one of the filter element(s) (e.g., the terminal filter element) may be comprised of cellulose acetate or polypropylene tow. The at least one filter element (e.g., the terminal filter element) may be comprised of activated charcoal. The at least one filter element (e.g., the terminal element) may be comprised of paper. The or each filter element may be at least partly (e.g., entirely) circumscribed with a plug wrap e.g., a paper plug wrap.

The terminal filter element (at the downstream end of the article/consumable) may be joined to the upstream elements forming the article/consumable by a circumscribing tipping layer e.g., a tipping paper layer. The tipping paper may have an axial length longer than the axial length of the terminal filter element such that the tipping paper completely circumscribes the terminal filter element plus the wrapping layer surrounding any adjacent upstream element.

In some embodiments, the article/consumable may comprise an aerosol-cooling element which is adapted to cool the aerosol generated from the aerosol-forming substrate (by heat exchange) before being inhaled by the user.

The article/consumable may comprise a spacer element that defines a space or cavity between the aerosol-forming substrate and the downstream end of the consumable.

The spacer element may comprise a cardboard tube. The spacer element may be circumscribed by the (paper) wrapping layer.

In a fourth aspect of the first mode, there is provided kit comprising the device according to the first aspect of the first mode and the tool according to the second aspect of the first mode.

The invention includes the combination of the aspects and preferred features of the first mode described except where such a combination is clearly impermissible or expressly avoided.

The skilled person will appreciate that except where mutually exclusive, a feature or parameter described in relation to any one of the above aspects of the first mode may be applied to any other aspect of the first mode. Furthermore, except where mutually exclusive, any feature or parameter of the first mode described herein may be applied to any aspect and/or combined with any other feature or parameter of the first mode described herein.

Second Mode: A Smoking Substitute Device Provided with a Cap Displacement Feature

At its most general, a second mode of the present invention relates to smoking substitute device provided with a cap displacement feature.

According to a first aspect of the second mode of the present invention, there is provided a smoking substitute device.

The device comprises a body and a heating element projecting from the body along a longitudinal axis. Further, the device comprises a cap, engageable with the body for at least partly enclosing the heating element. The cap is moveable away from the body along the longitudinal axis, whilst remaining engaged with the body, so as to define an aperture for accessing the heating element.

By providing a device comprising the cap, which is configured to move relative to the body along the longitudinal axis, and an aperture for accessing the heating element, cleaning of the heating element is facilitated, which may improve aerosol generation of the device. The user is provided with easy access to the heating element when the cap is moved away from the body. Furthermore, the cap remains engaged with the device, reducing the risk of losing the cap during cleaning and the cap is more easily replaced after cleaning the heating element.

The term “aperture” is intended to refer to a gap or an opening defined between the cap and the body, providing access to the heating element by the user.

Optional features will now be set out. These are applicable singly or in any combination with any aspect.

In some embodiments, the cap is defined with a cavity to receive a consumable. In this way, the cap may also function as a convenient means to eject the consumable after use.

In some embodiments, the heating element is configured to penetrate into at least a portion of the consumable.

Optionally, the cap is movable between a first position and a second position relative to the body, along the longitudinal axis, wherein the first position corresponds to a fully engaged condition of the cap with the body, and the second position corresponds to a lifted condition of the cap with respect to the body, defining the aperture.

In some embodiments, movement of the cap from the first position to the second position facilitates lifting of at least a portion of the consumable away from the heating element along the longitudinal axis.

Optionally, the displacement of the cap between the first position and the second position ranges from about 2 mm to about 15 mm, preferably from about 5 mm to about 8 mm. This provides a convenient range of movement which may be accomplished by a user with a single movement of e.g., the thumb, to move the cap between first and second positions.

Optionally, the cap may be engageable with the body by a sliding mechanism or a threading mechanism. This provides convenient and secure means to move the cap.

In some embodiments, the device comprises a retainer mechanism to retain the cap in one or more of the first position and the second position. In this way the cap is held more securely in a given position for ease of use, for example to facilitate cleaning of the heating element through the aperture when the cap is in the second position. In some embodiments, the device comprises a retainer mechanism to retain the cap in the second position.

In some embodiments, the retainer mechanism is configured to restrict movement of the cap beyond the second position. In other words, the second position may be a terminal position along the longitudinal motion of the cap away from the body. This provides a means to prevent inadvertent removal of the cap from the device, thereby reducing the risk of loss of the cap.

Optionally, the retainer mechanism is at least one of a detent mechanism and a magnetic lock mechanism. For example, the retainer mechanism may comprise a catch which holds the cap in one or more of the first and second position when the cap is in that position. The catch may be released when desired by action of the user. In some embodiments, the retainer mechanism comprises a magnetic catch, for example comprising magnets in the body and cap respectively between which a magnetic force of attraction exists which holds the cap in position. To move the cap out of the position the user must overcome this force.

In some embodiments, the retainer mechanism comprises one or more resilient members within the body of the device. In some embodiments, the one or more resilient members comprise a catch or detent which engages with a feature of the cap to prevent travel of the cap further from the body than the second position. In some embodiments the resilient members are deformable to disengage the catch or detent, allowing movement of the cap away from the body beyond the second position. In some embodiments, deformation of the resilient members is achieved by action of the user.

Optionally, the cap is removable from the body when in the second position by means of a tool. In this way the cap is not easily removed from the body so the risk of loss of the cap is low, but the user is able to remove the cap when needed through use of the removal tool. In some embodiments, the device comprises the tool, which is temporarily housed within the device and removable from the device as needed in order to be used to remove the cap. In some embodiments, the tool is adapted to deform the one or more resilient members described above when the tool is engaged, to disengage the catch or detent and permit removal of the cap by movement of the cap away from the body beyond the second position.

In some embodiments, movement of the cap towards the body beyond the first position is prevented by the abutment of a surface of the cap with a surface of the body. For example, an external rim of the cap may abut an external rim of the body. In some embodiments, the cap comprises an internal abutment surface which abuts a corresponding internal abutment surface of the body of the device in the first position.

Optionally, at least a portion of the cap is configured with a tactile finish to facilitate gripping of the cap for movement of the cap between the first position and the second position. In some embodiments, the tactile finish comprises a high-friction surface covering at least a portion of the cap. In some embodiments, the tactile finish comprises one or more raised protrusions, facilitating the engagement of a user's finger or thumb with the cap and the movement of the cap. In this way the user may more easily grip the cap to move it to inspect and/or clean the heating element through the aperture.

In some embodiments, the cap is biased into the second position. This facilitates movement of the cap into the second position for inspection/cleaning of the heating element. The cap and/or body of the device may comprise biasing means to bias the cap into the second position. For example, when the cap is biased into the second position the user need only disengage a retainer mechanism when the cap is in the first position and the cap will then naturally travel into the second position due to the biasing means. In some embodiments, the biasing means comprises a spring.

In some embodiments, the cap defines plurality of apertures when it is moved away from the body along the longitudinal axis. In some embodiments, two apertures are defined, wherein the apertures are defined on opposing sides of the device. This permits the user to access the heating element from either side of the device and further facilitates cleaning by allowing the user to access the heating element through a first aperture, and during cleaning push debris from the heating element out of the second aperture.

The device may comprise a body. An end of the body may be configured for engagement with an aerosol-forming article. For example, the body may be configured for engagement with a heated tobacco (HT) consumable (or heat-not-burn (HNB) consumable). The terms “heated tobacco” and “heat-not-burn” are used interchangeably herein to describe a consumable that is of the type that is heated rather than combusted (or are used interchangeably to describe a device for use with such a consumable). The device may comprise a cavity that is configured for receipt of at least a portion of the consumable (i.e., for engagement with the consumable). The aerosol-forming article may be of the type that comprises an aerosol former (e.g., carried by an aerosol-forming substrate).

The device may comprise a heater for heating the aerosol-forming article. The heater may comprise a heating element, which may be in the form of a rod that extends from the body of the device. The heating element may extend from the end of the body that is configured for engagement with the aerosol-forming article.

The heater (and thus the heating element) may be rigidly mounted to the body. The heating element may be elongate so as to define a longitudinal axis and may, for example, have a transverse profile (i.e., transverse to a longitudinal axis of the heating element) that is substantially circular (i.e., the heating element may be generally cylindrical). Alternatively, the heating element may have a transverse profile that is rectangular (i.e., the heater may be a “blade heater”). The heating element may alternatively be in the shape of a tube (i.e., the heater may be a “tube heater”). The heating element may take other forms (e.g., the heating element may have an elliptical transverse profile). The shape and/or size (e.g., diameter) of the transverse profile of the heating element may be generally consistent for the entire length (or substantially the entire length) of the heating element.

The heating element may be between 15 mm and 25 mm long, e.g., between 18 mm and 20 mm long, e.g., around 19 mm long. The heating element may have a diameter of between 1.5 mm and 2.5 mm, e.g., a diameter between 2 mm and 2.3 mm, e.g., a diameter of around 2.15 mm.

The heating element may be formed of ceramic. The heating element may comprise a core (e.g., a ceramic core) comprising Al2O3. The core of the heating element may have a diameter of 1.8 mm to 2.1 mm, e.g., between 1.9 mm and 2 mm. The heating element may comprise an outer layer (e.g., an outer ceramic layer) comprising Al2O3. The thickness of the outer layer may be between 160 μm and 220 μm, 5 e.g., between 170 μm and 190 μm, e.g., around 180 μm. The heating element may comprise a heating track, which may extend longitudinally along the heating element. The heating track may be sandwiched between the outer layer and the core of the heating element. The heating track may comprise tungsten and/or rhenium. The heating track may have a thickness of around 20 μm.

The heating element may be located in the cavity (of the device), and may extend (e.g., along a longitudinal axis) from an internal base of the cavity towards an opening of the cavity. The length of the heating element (i.e., along the longitudinal axis of the heater) may be less than the depth of the cavity. Hence, the heating element may extend for only a portion of the length of the cavity. That is, the heating element may not extend through (or beyond) the opening of the cavity.

The heating element may be configured for insertion into an aerosol-forming article (e.g., a HT consumable) when an aerosol-forming article is received in the cavity. In that respect, a distal end (i.e., distal from a base of the heating element where it is mounted to the device) of the heating element may comprise a tapered portion, which may facilitate insertion of the heating element into the aerosol-forming article. The heating element may fully penetrate an aerosol-forming article when the aerosol-forming article is received in the cavity. That is, the entire length, or substantially the entire length, of the heating element may be received in the aerosol-forming article.

The heating element may have a length that is less than, or substantially the same as, an axial length of an aerosol-forming substrate forming part of an aerosol-forming article (e.g., a HT consumable). Thus, when such an aerosol-forming article is engaged with the device, the heating element may only penetrate the aerosol-forming substrate, rather than other components of the aerosol-forming article. The heating element may penetrate the aerosol-forming substrate for substantially the entire axial length of the aerosol forming-substrate of the aerosol-forming article. Thus, heat may be transferred from (e.g., an outer circumferential surface of) the heating element to the surrounding aerosol-forming substrate, when penetrated by the heating element. That is, heat may be transferred radially outwardly (in the case of a cylindrical heating element) or e.g., radially inwardly (in the case of a tube heater).

Where the heater is a tube heater, the heating element of the tube heater may surround at least a portion of the cavity. When the portion of the aerosol-forming article is received in the cavity, the heating element may surround a portion of the aerosol-forming article (i.e., so as to heat that portion of the aerosol-forming article). In particular, the heating element may surround an aerosol forming substrate of the aerosol-forming article. That is, when an aerosol-forming article is engaged with the device, the aerosol forming substrate of the aerosol-forming article may be located adjacent an inner surface of the (tubular) heating element. When the heating element is activated, heat may be transferred radially inwardly from the inner surface of the heating element to heat the aerosol forming substrate.

The cavity may comprise a (e.g., circumferential) wall (or walls) and the (tubular) heating element may extend around at least a portion of the wall(s). In this way, the wall may be located between the inner surface of the heating element and an outer surface of the aerosol-forming article. The wall (or walls) of the cavity may be formed from a thermally conductive material (e.g., a metal) to allow heat conduction from the heating element to the aerosol-forming article. Thus, heat may be conducted from the heating element, through the cavity wall (or walls), to the aerosol-forming substrate of an aerosol-forming article received in the cavity.

In some embodiments, the device may comprise a cap disposed at the end of the body. The cap may be defined with a cavity for receiving an aerosol-forming article (i.e., consumable). The device comprises a heater having a heating element, the cap may at least partially enclose the heating element. The cap may be moveable between a first position and a second position relative to the body, along the longitudinal axis. The cap in the second position may define an aperture to facilitate access to the heating element, and in the first position the cap at least partially encloses the heating element. The cap may be slidably engaged with the body of the device, and may be slidable between the first and the second positions. The cap may be moved beyond the second position by a tool, which facilitates in disengaging the cap and the body.

In some embodiments, the cap may be movable between the first position and the second position by threading the cap with the housing or vice versa.

The cap may define at least a portion of the cavity of the device. That is, the cavity may be fully defined by the cap, or each of the cap and body may define a portion of the cavity. Where the cap fully defines the cavity, the cap may comprise an aperture for receipt of the heating element into the cavity (when the cap is in the closed position). The cap may comprise an opening to the cavity. The opening may be configured for receipt of at least a portion of an aerosol-forming article. That is, an aerosol-forming article may be inserted through the opening and into the cavity (so as to be engaged with the device).

The cap may be configured such that when an aerosol-forming article is engaged with the device (e.g., received in the cavity), only a portion of the aerosol-forming article is received in the cavity. That is, a portion of the aerosol-forming article (not received in the cavity) may protrude from (i.e., extend beyond) the opening. This (protruding) portion of the aerosol-forming article may be a terminal (e.g., mouth) end of the aerosol-forming article, which may be received in a user's mouth for the purpose of inhaling aerosol formed by the device.

The device may comprise a power source or may be connectable to a power source (e.g., a power source separate to the device). The power source may be electrically connectable to the heater. In that respect, altering (e.g., toggling) the electrical connection of the power source to the heater may affect a state of the heater. For example, toggling the electrical connection of the power source to the heater may toggle the heater between an on state and an off state. The power source may be a power store. For example, the power source may be a battery or rechargeable battery (e.g., a lithium-ion battery).

The device may comprise an input connection (e.g., a USB port, Micro USB port, USB-C port, etc.). The input connection may be configured for connection to an external source of electrical power, such as a mains electrical supply outlet. The input connection may, in some cases, be used as a substitute for an internal power source (e.g., battery or rechargeable battery). That is, the input connection may be electrically connectable to the heater (for providing power to the heater). Hence, in some forms, the input connection may form at least part of the power source of the device.

Where the power source comprises a rechargeable power source (such as a rechargeable battery), the input connection may be used to charge and recharge the power source.

The device may comprise a user interface (UI). In some embodiments the UI may include input means to receive operative commands from the user. The input means of the UI may allow the user to control at least one aspect of the operation of the device. In some embodiments the input means may comprise a power button to switch the device between an on state and an off state.

In some embodiments the UI may additionally or alternatively comprise output means to convey information to the user. In some embodiments the output means may comprise a light to indicate a condition of the device (and/or the aerosol-forming article) to the user. The condition of the device (and/or aerosol-forming article) indicated to the user may comprise a condition indicative of the operation of the heater. For example, the condition may comprise whether the heater is in an off state or an on state. In some embodiments, the UI unit may comprise at least one of a button, a display, a touchscreen, a switch, a light, and the like. For example, the output means may comprise one or more (e.g., two, three, four, etc.) light-emitting diodes (“LEDs”) that may be located on the body of the device.

The device may further comprise a puff sensor (e.g., airflow sensor), which form part of the input means of the UI. The puff sensor may be configured to detect a user drawing on an end (i.e., a terminal (mouth) end) of the aerosol-forming article. The puff sensor may, for example, be a pressure sensor or a microphone. The puff sensor may be configured to produce a signal indicative of a puff state. The signal may be indicative of the user drawing (an aerosol from the aerosol-forming article) such that it is e.g., in the form of a binary signal. Alternatively, or additionally, the signal may be indicative of a characteristic of the draw (e.g., a flow rate of the draw, length of time of the draw, etc.).

The device may comprise a controller, or may be connectable to a controller that may be configured to control at least one function of the device. The controller may comprise a microcontroller that may, e.g., be mounted on a printed circuit board (PCB). The controller may also comprise a memory, e.g., non-volatile memory. The memory may include instructions, which, when implemented, may cause the controller to perform certain tasks or steps of a method. Where the device comprises an input connection, the controller may be connected to the input connection.

The controller may be configured to control the operation of the heater (and e.g., the heating element). Thus, the controller may be configured to control vaporization of an aerosol forming part of an aerosol-forming article engaged with the device. The controller may be configured to control the voltage applied by power source to the heater. For example, the controller may be configured to toggle between applying a full output voltage (of the power source) to the heater and applying no voltage to the heater. Alternatively, or additionally, the control unit may implement a more complex heater control protocol.

The device may further comprise a voltage regulator to regulate the output voltage supplied by the power source to form a regulated voltage. The regulated voltage may subsequently be applied to the heater.

In some embodiments, where the device comprises a UI, the controller may be operatively connected to one or more components of the UI. The controller may be configured to receive command signals from an input means of the UI. The controller may be configured to control the heater in response to the command signals. For example, the controller may be configured to receive “on” and “off” command signals from the UI and, in response, may control the heater so as to be in a corresponding on or off state.

The controller may be configured to send output signals to a component of the UI. The UI may be configured to convey information to a user, via an output means, in response to such output signals (received from the controller). For example, where the device comprises one or more LEDs, the LEDs may be operatively connected to the controller. Hence, the controller may be configured to control the illumination of the LEDs (e.g., in response to an output signal). For example, the controller may be configured to control the illumination of the LEDs according to (e.g., an on or off) state of the heater.

Where the device comprises a sensor (e.g., a puff/airflow sensor), the controller may be operatively connected to the sensor. The controller may be configured to receive a signal from the sensor (e.g., indicative of a condition of the device and/or engaged aerosol-forming article). The controller may be configured to control the heater, or an aspect of the output means, based on the signal from the sensor.

The device may comprise a wireless interface configured to communicate wirelessly (e.g., via Bluetooth (e.g., a Bluetooth low-energy connection) or Wi-Fi) with an external device. Similarly, the input connection may be configured for wired connection to an external device so as to provide communication between the device and the external device.

The external device may be a mobile device. For example, the external device may be a smart phone, tablet, smart watch, or smart car. An application (e.g., app) may be installed on the external device (e.g., mobile device). The application may facilitate communication between the device and the external device via the wired or wireless connection.

The wireless or wired interface may be configured to transfer signals between the external device and the controller of the device. In this respect, the controller may control an aspect of the device in response to a signal received from an external device. Alternatively, or additionally, an external device may respond to a signal received from the device (e.g., from the controller of the device).

In a second aspect of the second mode, there is provided a system (e.g., a smoking substitute system) comprising a device according to the first aspect of the second mode and an aerosol-forming article. The aerosol-forming article may comprise an aerosol-forming substrate at an upstream end of the aerosol-forming article. The article may be in the form of a smoking substitute article, e.g., heated tobacco (HT) consumable (also known as a heat-not-burn (HNB) consumable).

As used herein, the terms “upstream” and “downstream” are intended to refer to the flow direction of the vapor/aerosol i.e., with the downstream end of the article/consumable being the mouth end or outlet where the aerosol exits the consumable for inhalation by the user. The upstream end of the article/consumable is the opposing end to the downstream end.

The aerosol-forming substrate is capable of being heated to release at least one volatile compound that can form an aerosol. The aerosol-forming substrate may be located at the upstream end of the article/consumable.

In order to generate an aerosol, the aerosol-forming substrate comprises at least one volatile compound that is intended to be vaporized/aerosolized and that may provide the user with a recreational and/or medicinal effect when inhaled. Suitable chemical and/or physiologically active volatile compounds include the group consisting of: nicotine, cocaine, caffeine, opiates and opioids, cathine and cathinone, kavalactones, mysticin, beta-carboline alkaloids, salvinorin A together with any combinations, functional equivalents to, and/or synthetic alternatives of the foregoing.

The aerosol-forming substrate may comprise plant material. The plant material may comprise least one plant material selected from the list including Amaranthus dubius, Arctostaphylos uva-ursi (Bearberry), Argemone mexicana, Amica, Artemisia vulgaris, Yellow Tees, Galea zacatechichi, Canavalia maritima (Baybean), Cecropia mexicana (Guamura), Cestrum noctumum, Cynoglossum virginianum (wild comfrey), Cytisus scoparius, Damiana, Entada rheedii, Eschscholzia californica (California Poppy), Fittonia albivenis, Hippobroma longiflora, Humulus japonica (Japanese Hops), Humulus lupulus (Hops), Lactuca virosa (Lettuce Opium), Laggera alata, Leonotis leonurus, Leonurus cardiaca (Motherwort), Leonurus sibiricus (Honeyweed), Lobelia cardinalis, Lobelia inflata (Indian-tobacco), Lobelia siphilitica, Nepeta cataria (Catnip), Nicotiana species (Tobacco), Nymphaea alba (White Lily), Nymphaea caerulea (Blue Lily), Opium poppy, Passiflora incamata (Passionflower), Pedicularis densiflora (Indian Warrior), Pedicularis groenlandica (Elephant's Head), Salvia divinorum, Salvia dorrii (Tobacco Sage), Salvia species (Sage), Scutellaria galericulata, Scutellaria lateriflora, Scutellaria nana, Scutellaria species (Skullcap), Sida acuta (Wireweed), Sida rhombifolia, Silene capensis, Syzygium aromaticum (Clove), Tagetes lucida (Mexican Tarragon), Tarchonanthus camphoratus, Tumera diffusa (Damiana), Verbascum (Mullein), Zamia latifolia (Maconha Brava) together with any combinations, functional equivalents to, and/or synthetic alternatives of the foregoing.

The plant material may be tobacco. Any type of tobacco may be used. This includes, but is not limited to, flue-cured tobacco, burley tobacco, Maryland Tobacco, dark-air cured tobacco, oriental tobacco, dark-fired tobacco, perique tobacco and rustica tobacco. This also includes blends of the above-mentioned tobaccos.

The tobacco may comprise one or more of leaf tobacco, stem tobacco, tobacco powder, tobacco dust, tobacco derivatives, expanded tobacco, homogenized tobacco, shredded tobacco, extruded tobacco, cut rag tobacco and/or reconstituted tobacco (e.g., slurry recon or paper recon).

The aerosol-forming substrate may comprise a gathered sheet of homogenized (e.g., paper/slurry recon) tobacco or gathered shreds/strips formed from such a sheet.

The aerosol-forming substrate may comprise one or more additives selected from humectants, flavorants, fillers, aqueous/non-aqueous solvents and binders.

The flavorant may be provided in solid or liquid form. It may include menthol, licorice, chocolate, fruit flavor (including e.g., citrus, cherry etc.), vanilla, spice (e.g., ginger, cinnamon) and tobacco flavor. The flavorant may be evenly dispersed throughout the aerosol-forming substrate or may be provided in isolated locations and/or varying concentrations throughout the aerosol-forming substrate.

The aerosol-forming substrate may be formed in a substantially cylindrical shape such that the article/consumable resembles a conventional cigarette. It may have a diameter of between 5 and 10 mm e.g., between 6 and 9 mm or 6 and 8 mm e.g., around 7 mm. It may have an axial length of between 10 and 15 mm e.g., between 11 and 14 mm such as around 12 or 13 mm.

The article/consumable may comprise at least one filter element. There may be a terminal filter element at the downstream/mouth end of the article/consumable.

The or at least one of the filter element(s) (e.g., the terminal filter element) may be comprised of cellulose acetate or polypropylene tow. The at least one filter element (e.g., the terminal filter element) may be comprised of activated charcoal. The at least one filter element (e.g., the terminal element) may be comprised of paper. The or each filter element may be at least partly (e.g., entirely) circumscribed with a plug wrap e.g., a paper plug wrap.

The terminal filter element (at the downstream end of the article/consumable) may be joined to the upstream elements forming the article/consumable by a circumscribing tipping layer e.g., a tipping paper layer. The tipping paper may have an axial length longer than the axial length of the terminal filter element such that the tipping paper completely circumscribes the terminal filter element plus the wrapping layer surrounding any adjacent upstream element.

In some embodiments, the article/consumable may comprise an aerosol-cooling element which is adapted to cool the aerosol generated from the aerosol-forming substrate (by heat exchange) before being inhaled by the user.

The article/consumable may comprise a spacer element that defines a space or cavity between the aerosol-forming substrate and the downstream end of the consumable. The spacer element may comprise a cardboard tube. The spacer element may be circumscribed by the (paper) wrapping layer.

The invention includes the combination of the aspects and preferred features of the second mode described except where such a combination is clearly impermissible or expressly avoided.

The skilled person will appreciate that except where mutually exclusive, a feature or parameter described in relation to any one of the above aspects of the second mode may be applied to any other aspect of the second mode. Furthermore, except where mutually exclusive, any feature or parameter described herein may be applied to any aspect and/or combined with any other feature or parameter described herein.

### SUMMARY OF THE FIGURES

So that the invention may be understood, and so that further aspects and features thereof may be appreciated, embodiments illustrating the principles of the invention will now be discussed in further detail with reference to the accompanying figures, in which:

FIG. 1 is a schematic of a smoking substitute system of the first mode.

FIG. 2A is a front view of a first embodiment of the first mode of a smoking substitute system with the consumable engaged with the device.

FIG. 2B is a front view of the first embodiment of the first mode of the smoking substitute system with the consumable disengaged from the device.

FIG. 2C is a section view of the consumable of the first embodiment of the first mode of the smoking substitute system.

FIG. 2D is a detailed view of an end of the device of the first embodiment of the first mode of the smoking substitute system.

FIG. 2E is a section view of the first embodiment of the first mode of the smoking substitute system.

FIG. 3 is perspective view of the first embodiment of the first mode of the substitute smoking system with a cap in a second position.

FIG. 4A is section view of the first embodiment of the first mode of the smoking substitute system with the cap in a first position.

FIG. 4B is section view of the first embodiment of the first mode of the smoking substitute system with the cap in the second position.

FIG. 5A is a perspective view of the tool in accordance with an embodiment of the first mode.

FIG. 5B is a perspective view of the tool of FIG. 5A with a first cover removed.

FIG. 5C is a perspective view of the tool of FIG. 5A with a second cover removed.

FIG. 6A is a section view of the first embodiment of the first mode with the tool inserted in the cavity in insertion position.

FIG. 6B is a section view of the first embodiment of the first mode with the tool inserted in the cavity in unlocking position.

FIG. 7A-E illustrates stages of using the tool to separate the cap from the body of the smoking substitute device of the first embodiment of the first mode.

FIG. 8A is a schematic of a smoking substitute system of the second mode.

FIG. 8B is a schematic of a variation of the smoking substitute system of FIG. 8A.

FIG. 9A is a front view of a first embodiment of the second mode of a smoking substitute system with the consumable engaged with the device.

FIG. 9B is a front view of the first embodiment of the second mode of the smoking substitute system with the consumable disengaged from the device.

FIG. 9C is a section view of the consumable of the first embodiment of the second mode of the smoking substitute system.

FIG. 9D is a detailed view of an end of the device of the first embodiment of the second mode of the smoking substitute system.

FIG. 9E is a section view of the first embodiment of the second mode of the substitute smoking system.

FIG. 9F is a sectional view of the first embodiment of the second mode of the device, showing the cap engaged with the body.

FIG. 9G is a perspective view of the first embodiment of the second mode of the device showing the cap in the second position.

FIG. 9H is a sectional view of the first embodiment of the second mode of the device, showing the cap in the second position.

FIG. 10 is a detailed view of the first embodiment of the second mode of the device, with a tool inserted into the cap.

FIG. 11 is a detailed view of the first embodiment of the second mode of the device, with the cap disengaged from the body.

### DETAILED DESCRIPTION OF THE FIGURES

First Mode: A Smoking Substitute System with a Smoking Substitute Device Having a Cap Movable Between Two Positions

Aspects and embodiments of the first mode of the present invention will now be discussed with reference to the accompanying figures. Further aspects and embodiments will be apparent to those skilled in the art. All documents mentioned in this text are incorporated herein by reference.

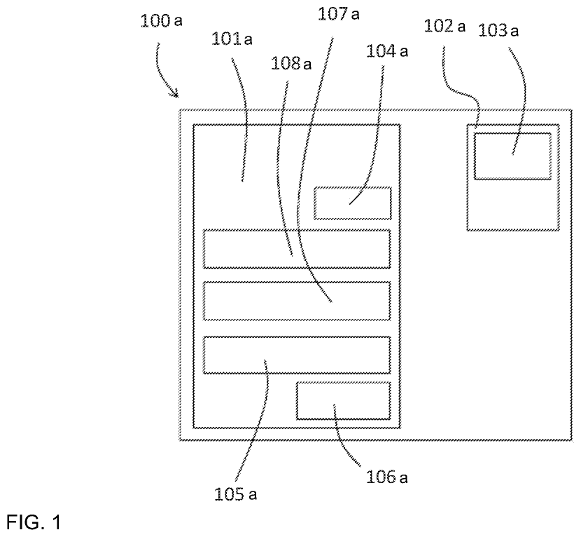

FIG. 1 is a schematic providing a general overview of a smoking substitute system 100a. The system 100a includes a substitute smoking device 101a and an aerosol-forming article in the form of a consumable 102a, which comprises an aerosol former 103a. The system is configured to vaporize the aerosol former by heating the aerosol former 103a (so as to form a vapor/aerosol for inhalation by a user).

In the illustrated system, the heater 104a forms part of the device 101a and is configured to heat the aerosol former 103a. Heat from the heater 104a vaporizes the aerosol former 103a to produce a vapor. The vapor subsequently condenses to form an aerosol, which is ultimately inhaled by the user.

The system 100a further comprises a power source 105a that forms part of the device 101a. In other embodiments the power source 105a may be external to (but connectable to) the device 101a. The power source 105a is electrically connectable to the heater 104a such that it is able to supply power to the heater 104a (i.e., for the purpose of heating the aerosol former 103a). Thus, control of the electrical connection of the power source 105a to the heater 104a provides control of the state of the heater 104a. The power source 105a may be a power store, for example a battery or rechargeable battery (e.g., a lithium-ion battery).

The system 100a further comprises an I/O module comprising a connector 106a (e.g., in the form of a USB port, Micro USB port, USB-C port, etc.). The connector 106a is configured for connection to an external source of electrical power, e.g., a mains electrical supply outlet. The connector 106a may be used in substitution for the power source 105a. That is the connector 106a may be electrically connectable to the heater 104a so as to supply electricity to the heater 104a. In such embodiments, the device may not include a power source, and the power source of the system may instead comprise the connector 106a and an external source of electrical power (to which the connector 106a provides electrical connection).

In some embodiments, the connector 106a may be used to charge and recharge the power source 105a where the power source 104a includes a rechargeable battery.

The system 100a also comprises a user interface (UI) 107a. Although not shown, the UI 107a may include input means to receive commands from a user. The input means of the UI 107a allows the user to control at least one aspect of the operation of the system 100a. The input means may, for example, be in the form of a button, touchscreen, switch, microphone, etc.

The UI 107a also comprises output means to convey information to the user. The output means may, for example, comprise lights (e.g., LEDs), a display screen, speaker, vibration generator, etc.

The system 100a further comprises a controller 108a that is configured to control at least one function of the device 101a. In the illustrated embodiment, the controller 108a is a component of the device 101a, but in other embodiments may be separate from (but connectable to) the device 101a. The controller 108a is configured to control the operation of the heater 104a and, for example, may be configured to control the voltage applied from the power source 105a to the heater 104a. The controller 108a may be configured to toggle the supply of power to the heater 105a between an on state, in which the full output voltage of the power source 105a is applied to the heater 104a, and an off state, in which the no voltage is applied to the heater 104a.

Although not shown, the system 100a may also comprise a voltage regulator to regulate the output voltage from the power source 105a to form a regulated voltage. The regulated voltage may then be applied to the heater 104a.

In addition to being connected to the heater 104a, the controller 108a is operatively connected to the UI 107a. Thus, the controller 108a may receive an input signal from the input means of the UI 107a. Similarly, the controller 108a may transmit output signals to the UI 107a. In response, the output means of the UI 107a may convey information, based on the output signals, to a user.

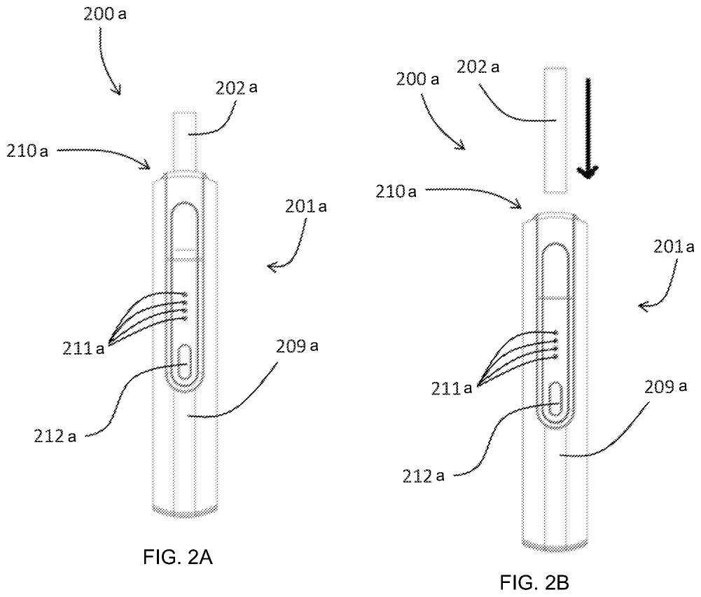

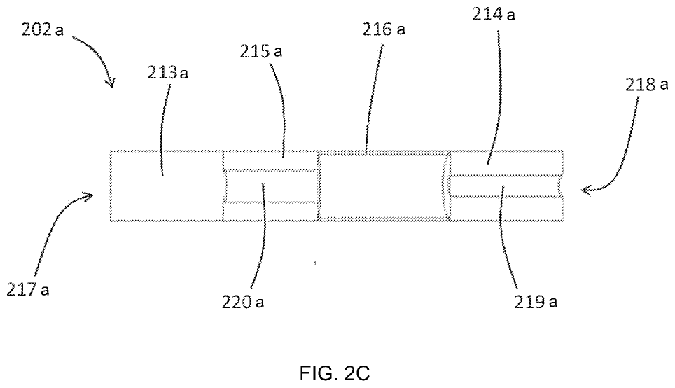

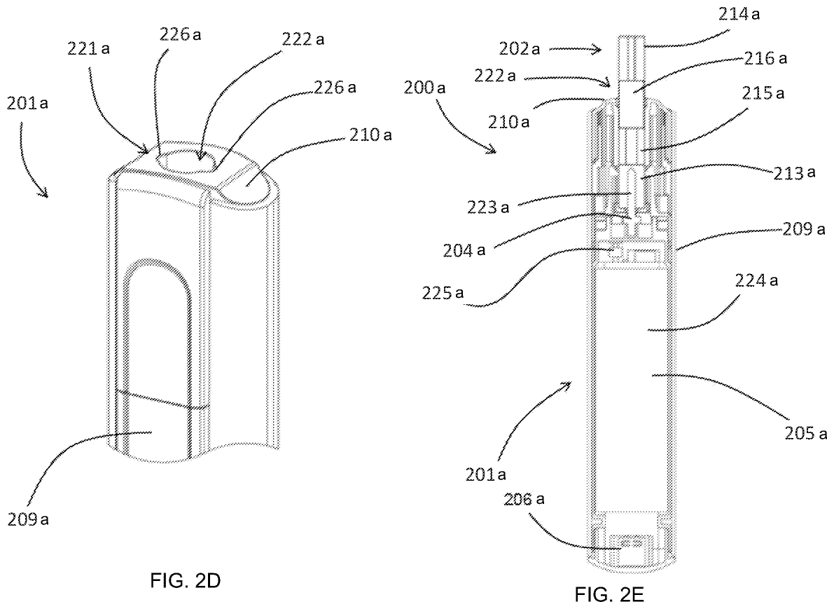

FIG. 2A and FIG. 2B illustrate a heated-tobacco (HT) smoking substitute system 200a. The system 200a is an example of the systems 100a, described in relation to FIG. 1. System 200a includes an HT device 201a and an HT consumable 202a. The description of FIG. 1 above is applicable to the system 200a of FIG. 2A and FIG. 2B, and will thus not be repeated.

The device 201a and the consumable 202a are configured such that the consumable 202a can be engaged with the device 201a. FIG. 2A shows the device 201a and the consumable 202a in an engaged state, whilst FIG. 2B shows the device 201a and the consumable 202a in a disengaged state.

The device 201a comprises a body 209a and cap 210a. In use the cap 209a is engaged at an end of the body 209a. Although not apparent from the figures, the cap 210a is moveable relative to the body 209a. In particular, the cap 210a is sliceable and can slide along a longitudinal axis of the body 209a.

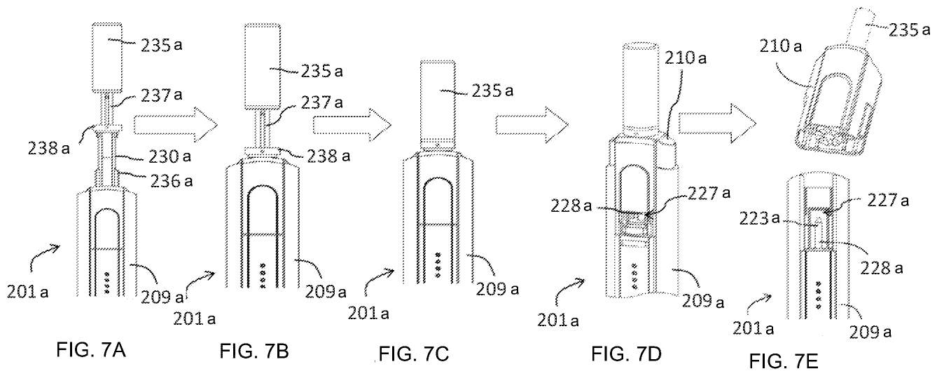

As shown in FIG. 7E, the body 209a defines a transverse cavity 227a extending orthogonal to the longitudinal axis of the body 209a. The transverse cavity 227a opens through and extends from a first side wall of the body 209a towards and surrounding at least a portion of the heating element. The transverse cavity 227a is located on the body 209a such that at least a base 228d of the heating element is juxtaposed with the transverse cavity 227a. The transverse cavity 227a extends from a first side wall of the body 209a to and through a second side wall opposite to the first side wall of the body 209a. That is, the transverse cavity 227a forms a through hole extending through the body 209a.

The device 201a comprises an output means (forming part of the UI of the device 201a) in the form of a plurality of light-emitting diodes (LEDs) 211a arranged linearly along the longitudinal axis of the device 201a and on an outer surface of the body 209a of the device 201a. A button 212a is also arranged on an outer surface of the body 209a of the device 201a and is axially spaced (i.e., along the longitudinal axis) from the plurality of LEDs 211a.

FIG. 2C show a detailed section view of the consumable 202a of the system 200a. The consumable 202a generally resembles a cigarette. In that respect, the consumable 202a has a generally cylindrical form with a diameter of 7 mm and an axial length of 70 mm. The consumable 202a comprises an aerosol forming substrate 213a, a terminal filter element 214a, an upstream filter element 215a and a spacer element 216a. In other embodiments, the consumable may further comprise a cooling element. A cooling element may exchange heat with vapor that is formed by the aerosol-forming substrate 213a in order to cool the vapor so as to facilitate condensation of the vapor.

The aerosol-forming substrate 213a is substantially cylindrical and is located at an upstream end 217a of the consumable 202a, and comprises the aerosol former of the system 200a. In that respect, the aerosol forming substrate 213a is configured to be heated by the device 201a to release a vapor. The released vapor is subsequently entrained in an airflow flowing through the aerosol-forming substrate 213a. The airflow is produced by the action of the user drawing on a downstream 218a (i.e., terminal or mouth end) of the consumable 202a.

In the present embodiment, the aerosol forming substrate 213a comprises tobacco material that may, for example, include any suitable parts of the tobacco plant (e.g., leaves, stems, roots, bark, seeds and flowers). The tobacco may comprise one or more of leaf tobacco, stem tobacco, tobacco powder, tobacco dust, tobacco derivatives, expanded tobacco, homogenized tobacco, shredded tobacco, extruded tobacco, cut rag tobacco and/or reconstituted tobacco (e.g., slurry recon or paper recon). For example, the aerosol-forming substrate 213a may comprise a gathered sheet of homogenized (e.g., paper/slurry recon) tobacco or gathered shreds/strips formed from such a sheet.

In order to generate an aerosol, the aerosol forming substrate 213a comprises at least one volatile compound that is intended to be vaporized/aerosolized and that may provide the user with a recreational and/or medicinal effect when inhaled. The aerosol-forming substrate 213a may further comprise one or more additives. For example, such additives may be in the form of humectants (e.g., propylene glycol and/or vegetable glycerin), flavorants, fillers, aqueous/non-aqueous solvents and/or binders.

The terminal filter element 214a is also substantially cylindrical, and is located downstream of the aerosol forming substrate 213a at the downstream end 218a of the consumable 202a. The terminal filter element 214a is in the form of a hollow bore filter element having a bore 219a (e.g., for airflow) formed therethrough. The diameter of the bore 219a is 2 mm. The terminal filter element 214a is formed of a porous (e.g., monoacetate) filter material. As set forth above, the downstream end 218a of the consumable 202a (i.e., where the terminal filter 214a is located) forms a mouthpiece portion of the consumable 202a upon which the user draws. Airflow is drawn from the upstream end 217a, thorough the components of the consumable 202a, and out of the downstream end 218a. The airflow is driven by the user drawing on the downstream end 218a (i.e., the mouthpiece portion) of the consumable 202a.

The upstream filter element 215a is located axially adjacent to the aerosol-forming substrate 213a, between the aerosol-forming substrate 213a and the terminal filter element 214a. Like the terminal filter 214a, the upstream filter element 215a is in the form of a hollow bore filter element, such that it has a bore 220a extending axially therethrough. In this way, the upstream filter 215a may act as an airflow restrictor. The upstream filter element 215a is formed of a porous (e.g., monoacetate) filter material. The bore 220a of the upstream filter element 214a has a larger diameter (3 mm) than the terminal filter element 214a.

The spacer 216a is in the form of a cardboard tube, which defines a cavity or chamber between the upstream filter element 215a and the terminal filter element 214a. The spacer 216a acts to allow both cooling and mixing of the vapor/aerosol from the aerosol-forming substrate 213a. The spacer has an external diameter of 7 mm and an axial length of 14 mm.

Although not apparent from the figure, the aerosol-forming substrate 213a, upstream filter 215a and spacer 216a are circumscribed by a paper wrapping layer. The terminal filter 214a is circumscribed by a tipping layer that also circumscribes a portion of the paper wrapping layer (so as to connect the terminal filter 214a to the remaining components of the consumable 202a). The upstream filter 215a and terminal filter 214a are circumscribed by further wrapping layers in the form of plug wraps.

Returning now to the device 201a, FIG. 2D illustrates a detailed view of the end of the device 201a that is configured to engage with the consumable 202a. The cap 210a of the device 201a includes an opening 221a to an internal cavity 222a (more apparent from FIG. 2D) defined by the cap 210a. The opening 221a and the cavity 222a are formed so as to receive at least a portion of the consumable 202a. During engagement of the consumable 202a with the device 201a, a portion of the consumable 202a is received through the opening 221a and into the cavity 222a. After engagement (see FIG. 2B), the downstream end 218a of the consumable 202a protrudes from the opening 221a and thus also protrudes from the device 201a. The opening 221a includes laterally disposed notches 226a. When a consumable 202a is received in the opening 221a, these notches 226a remain open and could, for example, be used for retaining a cover in order to cover the end of the device 201a.

FIG. 2E shows a cross section through a central longitudinal plane through the device 201a. The device 201a is shown with the consumable 202a engaged therewith. Further, as illustrated, at least one locking arm 229a extends from the body 209a. The locking arms 229a lock or retain the cap 210a with the body 209a. In the embodiment as illustrated, two locking arms 229a are present. In an embodiment, any suitable number of locking arms 229a may be provided. The locking arms 229a extend substantially along the longitudinal axis of the body 209a as shown. The locking arms 229a are provided with a locking protrusion 231a at a distal end, i.e., an end distal from an end of the locking arm 229a that is connected to the body 209a. The locking protrusion 231a extend transversely to the longitudinal axis of the body 209a. The locking arms 229a are positioned such that when the cap 210a is mounted on the body 209a, the locking arms 229a engage the cap 210a to retain the cap 210a on the body 209a.

In the embodiment as shown, the cap 210a may be provided with a slot 232a extending along the longitudinal axis of the body 209a (when the cap 210a is retained on the body 209a), and the locking protrusions 231a may be configured or positioned to engage the slot 232a. The slot 232a may be elongated such that the cap 210a may be moved or slid relative to the body 209a along the longitudinal axis of the body 209a. The locking protrusion 231a may have an abutment surface 233d to engage a peripheral surface 234d of the cap 210a that defines the slot 232a. The abutment surface 233d may block movement of the cap 210a in one direction by abutting the peripheral surface 234d to retain or lock the cap 210a with the body 209a.

The cap 210a is movable between a first position and a second position. FIG. 2A, FIG. 2B, FIG. 2D, and FIG. 4A illustrate the device 200a with the cap 210a in the first position. When the cap 210a is in the first position, the cap 210a conceals the heating element 223a, as illustrated. In the first position, the cap 210a completely covers the transverse cavity 227a to conceal the heating element 223a.

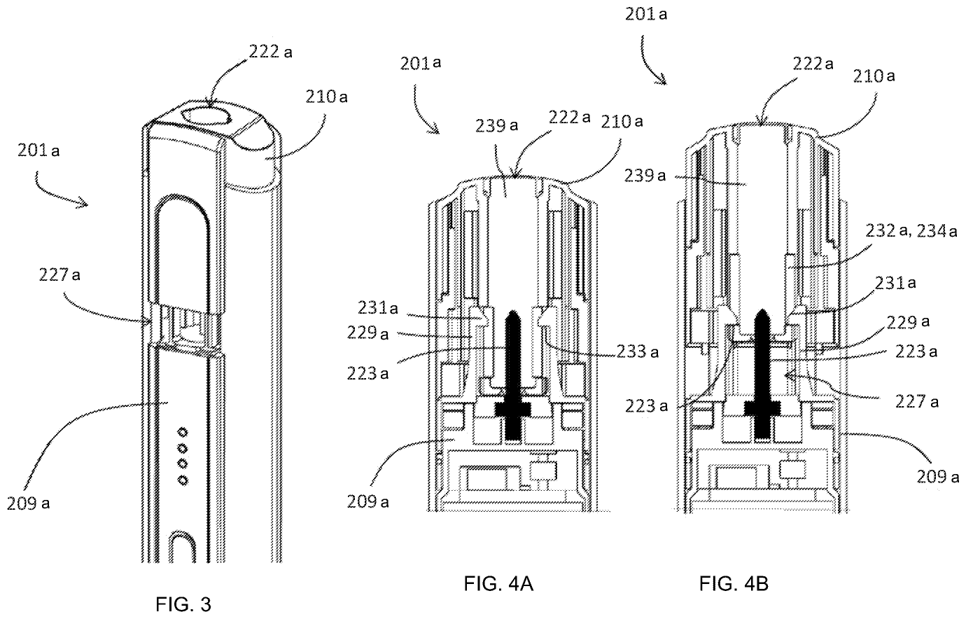

FIG. 3 and FIG. 4B illustrate the device 200a with the cap 210a in the second position. When the cap 210a is in the second position, the cap 210a at least partially exposes the heating element 223a. In the second position, the cap 210a does not cover the transverse cavity 227a to partially expose the heating element 223a. When the heating element 223a is partially exposed, the heating element 223a may be examined visually to ascertain if cleaning of the heating element 223a is required. If required, when the cap 210a is in the second position, the heating element 223a may be cleaned by blowing air through the opening or simply shaking, tilting and or tapping the device gently to dislodge and remove loose debris. In the second position, the abutment surface 233d of the cap 210a may abut the peripheral surface 234d as discussed in the foregoing description.

The device 201a comprises a heater 204a comprising heating element 223a. The heater 204a forms part of the body 209a of the device 201a and is rigidly mounted to the body 209a. In the illustrated embodiment, the heater 204a is a rod heater with a heating element 223a having a circular transverse profile. In other embodiments the heater may be in the form of a blade heater (e.g., heating element with a rectangular transverse profile) or a tube heater (e.g., heating element with a tubular form).

The heating element 223a of the heater 204a projects from an internal base of the cavity 222a along a longitudinal axis towards the opening 221a. As is apparent from the figure, the length (i.e., along the longitudinal axis) of the heating element is less than a depth of the cavity 222a. In this way, the heating element 223a does not protrude from or extend beyond the opening 221a.

When the consumable 202a is received in the cavity 222a (as is shown in FIG. 2E), the heating element 223a penetrates the aerosol-forming substrate 213a of the consumable 202a. In particular, the heating element 223a extends for nearly the entire axial length of the aerosol-forming substrate 213a when inserted therein. Thus, when the heater 204a is activated, heat is transferred radially from an outer circumferential surface of the heating element 223a to the aerosol-forming substrate 213a.

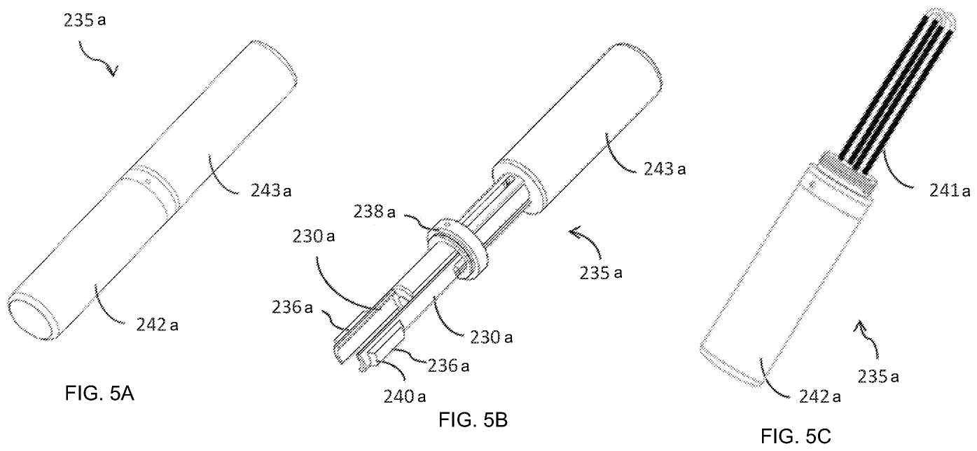

The smoking substitute system of the present disclosure may further include a tool 235a for separation of the cap 210a from the body 209a. The tool 235a may be configured to displace the locking arms 229a to enable separation of the cap 210a from the body 209a. FIG. 5A, FIG. 5B, and FIG. 5C illustrate a tool 235a in accordance with an embodiment. The tool 235a has at least one unlocking arm 230a. In the embodiment as illustrated, two unlocking arms 230a are provided. The number of unlocking arms 230a may be provided as required. In an embodiment, the number of unlocking arms 230a may correspond to the number of locking arms 229a. The unlocking arms 230a are adapted to engage the locking arms 229a to displace the locking arms 229a for separating the cap 210a from the body 209a. Each unlocking arm 230a may be provided with an unlocking protrusion 236a. The unlocking protrusion 236a may extend in a direction orthogonal to the longitudinal axis of the unlocking arm 230a. The unlocking protrusions 236a are adapted to engage the locking protrusions 231a to displace the locking protrusions 231a for releasing the cap 210a from the body 209a.

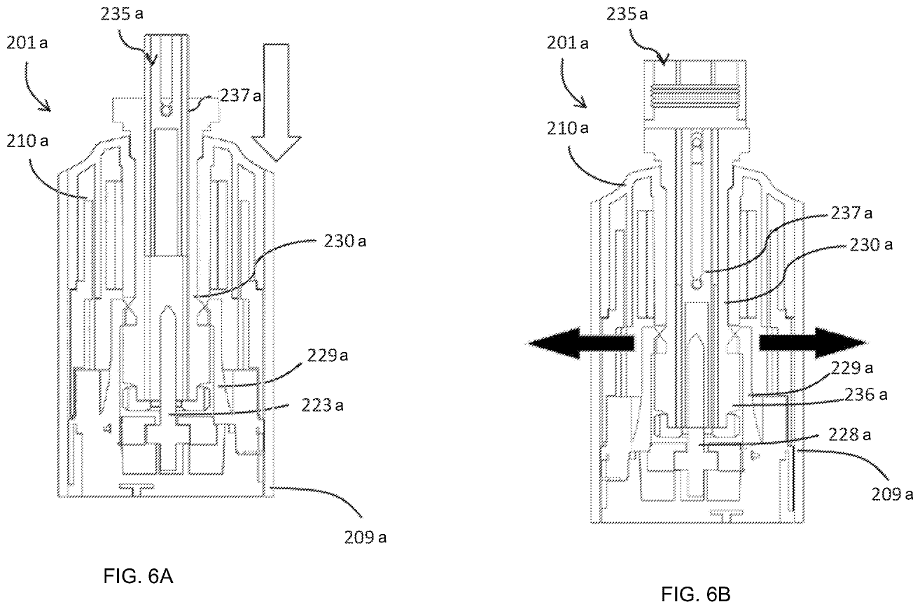

The tool 235a may include a central rod 237a. A collar 238a may be positioned concentrically around the central rod 237a. The collar 238a may be placed movably on the rod such that the collar 238a moves relative to the central rod 237a along a longitudinal axis of the central rod 237a. The unlocking arms 230a may extend from the collar 238a along the longitudinal axis of the central rod 237a. The collar 238a may be movable on the central rod 237a between an insertion position and an unlocking position. In the insertion position, the central rod 237a may be kept away from the unlocking protrusions 236a and the unlocking arms 230a may flex radially inwards relative to the longitudinal axis of the central rod 237a. In FIG. 5B and FIG. 6A, the collar 238a is shown in the insertion position. In the unlocking position, the central rod 237a moves in juxtaposition with the unlocking protrusions 236a to prevent flexing of the unlocking arms 230a in a direction radially inwards relative to the longitudinal axis of the central rod 237a. FIG. 6B illustrates the collar 238a in the unlocking position. Suitable provision may be provided on the collar 238a and the rod to enable and/or guide movement of the collar 238a between the insertion position and the unlocking position. The collar 238a may be biased to move towards the insertion position using any suitable means such as a coil spring.

The tool 235a may be configured for insertion into the cavity 222a as shown through FIGS. 7A-E. The unlocking protrusions 236a are configured such that when the unlocking arms 230a are inserted into the cavity 222a, the unlocking arm 230a displaces the locking arms 229a to release engagement of the locking arms 229a from the slots 232a. In the embodiment as illustrated, the unlocking protrusions 236a are configured such that when inserted into the cavity 222a, the unlocking protrusions 236a enter the slots 232a defined in the cap 210a to displace the locking protrusions 231a, in order to dislodge the cap 210a from the body 209a. The unlocking protrusions 236a may have dimensions that interfere with the width of the cavity 222a. Thus, in order to allow insertion of the unlocking arms 230a in the cavity 222a, in the insertion position, the central rod 237a is away from the distal ends of the unlocking arms 230a to allow the distal ends of the unlocking arms 230a to flex radially inwards to enable insertion of the unlocking arms 230a with the unlocking protrusions 236a into the cavity 222a. The flexing may be achieved when the unlocking protrusions 236a abut and slide against an inner surface 239a of cap 210a defining the internal cavity 222a. The unlocking protrusions 236a, as shown in the embodiment illustrated, may be provided with tapered surfaces 240a to guide the flexing movement of the unlocking arms 230a in and out from the cavity 222a and the slots 232a. FIG. 7A shows the tool 235a being inserted in the cavity 222a with the collar 238a in the insertion position.

In the initial stage, the tool 235a may be pushed towards the body 209a (as indicated by directional arrow in FIG. 6A) to insert the unlocking arms 230a into the cavity 222a until the collar 238a abuts the opening of the cavity 222a as shown in FIG. 6A and FIG. 7B. At this stage, as shown in FIG. 6A, the unlocking arms 230a enter the slots 232a defined in the cap 210a. At this stage, the unlocking protrusions 236a may not completely displace the locking protrusions 231a as required for separation of the cap 210a. Further, the central rod 237a may be pushed into the cavity 222a to move the collar 238a (relative to the central rod 237a) to the unlocking position as shown in FIG. 26c. On pushing the central rod 237a, the unlocking protrusion 236a may be pushed radially outward to enter the slots 232a properly and occupy the slot 232a as shown in FIG. 6B, to displace and move the locking protrusions 231a radially outward (shown by the arrows) to remove them from the slots 232a. After this, the cap 210a along with tool 235a may be pulled away from the body 209a to separate the cap 210a from the body 209a as shown in FIG. 7D. FIG. 7E illustrates cap 210a completely separated from the body 209a.

The tool 235a may further have a cleaning means for cleaning the heating element 223a. The cleaning means may be in form of cleaning bristles 241a as shown in FIG. 5C. The cleaning bristles 241a may extend from the central rod 237a in a direction opposite to the direction of extension of the unlocking arms 230a, as shown in FIG. 5C. The cleaning bristles 241a may be rubbed on the outer surface of the heating element 223a to clean or scrap off any debris or residuals from the heating element 223a.

The tool 235a may include a first cover 242d to cover the unlocking arms 230a when not in use.

Further, a second cover 243d may be provided to cover the cleaning bristles 241a when not in use. The covers 242d, 243d may be designed such that the tool 235a may visually resemble a consumable for the smoking substitute system. Suitable provisions may be provided to retain the cover on the tool 235a.

The device 202a further comprises an electronics cavity 224a. A power source, in the form of a rechargeable battery 205a (a lithium-ion battery), is located in electronics cavity 224a.

The device 202a includes a connector (i.e., forming part of an IO module of the device 201a) in the form of a USB port 206a. The connector may alternatively be, for example, a micro-USB port or a USB-C port for examples. The USB port 206a may be used to recharge the rechargeable battery 205a.

The device 202a includes a controller (not shown) located in the electronics cavity 224a. The controller comprises a microcontroller mounted on a printed circuit board (PCB). The USB port 206a is also connected to the controller 208a (i.e., connected to the PCB and microcontroller).

The controller 208a is configured to control at least one function of the device 202a. For example, the controller 208a is configured to control the operation of the heater 204a. Such control of the operation of the heater 204a may be accomplished by the controller toggling the electrical connection of the rechargeable battery 205a to the heater 204a. For example, the controller 208a is configured to control the heater 204a in response to a user depressing the button 212a. Depressing the button 212a may cause the controller to allow a voltage (from the rechargeable battery 205a) to be applied to the heater 204a (so as to cause the heating element 223a to be heated).

The controller is also configured to control the LEDs 211a in response to (e.g., a detected) a condition of the device 201a or the consumable 202a. For example, the controller may control the LEDs to indicate whether the device 201a is in an on state or an off state (e.g., one or more of the LEDs may be illuminated by the controller when the device is in an on state).

The device 202a comprises a further input means (i.e., in addition to the button 212a) in the form of a puff sensor 225a. The puff sensor 225a is configured to detect a user drawing (i.e., inhaling) at the downstream end 218a of the consumable 202a. The puff sensor 225a may, for example, be in the form of a pressure sensor, flowmeter or a microphone. The puff sensor 225a is operatively connected to the controller 208a in the electronics cavity 224a, such that a signal from the puff sensor 225a, indicative of a puff state (i.e., drawing or not drawing), forms an input to the controller 208a (and can thus be responded to by the controller 208a).

Second Mode: A Smoking Substitute Device Provided with a Cap Displacement Feature

Aspects and embodiments of the second mode of the present invention will now be discussed with reference to the accompanying figures. Further aspects and embodiments will be apparent to those skilled in the art. All documents mentioned in this text are incorporated herein by reference.

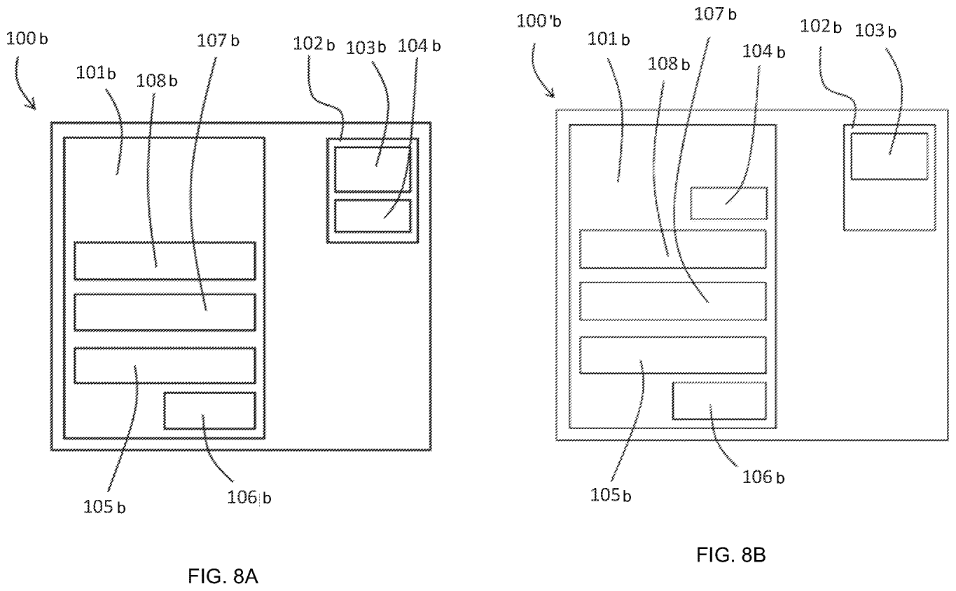

FIG. 8A is a schematic providing a general overview of a smoking substitute system 100b. The system 100b includes a substitute smoking device 101b and an aerosol-forming article in the form of a consumable 102b, which comprises an aerosol former 103b. The system is configured to vaporize the aerosol former by heating the aerosol former 103b (so as to form a vapor/aerosol for inhalation by a user).

In the illustrated system, the heater 104b forms part of the consumable 102b and is configured to heat the aerosol former 103b. In this variation, the heater 104b is electrically connectable to the power source 105b, for example, when the consumable 102b is engaged with the device 101b. Heat from the heater 104b vaporizes the aerosol former 103b to produce a vapor. The vapor subsequently condenses to form an aerosol, which is ultimately inhaled by the user.

The system 100b further comprises a power source 105b that forms part of the device 101b. In other embodiments the power source 105b may be external to (but connectable to) the device 101b. The power source 105b is electrically connectable to the heater 104b such that it is able to supply power to the heater 104b (i.e., for the purpose of heating the aerosol former 103b). Thus, control of the electrical connection of the power source 105b to the heater 104b provides control of the state of the heater 104b. The power source 105b may be a power store, for example a battery or rechargeable battery (e.g., a lithium-ion battery).

The system 100b further comprises an I/O module comprising a connector 106b (e.g., in the form of a USB port, Micro USB port, USB-C port, etc.). The connector 106b is configured for connection to an external source of electrical power, e.g., a mains electrical supply outlet. The connector 106b may be used in substitution for the power source 105b. That is the connector 106b may be electrically connectable to the heater 104b so as to supply electricity to the heater 104b. In such embodiments, the device may not include a power source, and the power source of the system may instead comprise the connector 106b and an external source of electrical power (to which the connector 106b provides electrical connection).

In some embodiments, the connector 106b may be used to charge and recharge the power source 105b where the power source 105b includes a rechargeable battery.

The system 100b also comprises a user interface (UI) 107b. Although not shown, the UI 107b may include input means to receive commands from a user. The input means of the UI 107b allows the user to control at least one aspect of the operation of the system 100b. The input means may, for example, be in the form of a button, touchscreen, switch, microphone, etc.

The UI 107b also comprises output means to convey information to the user. The output means may, for example, comprise lights (e.g., LEDs), a display screen, speaker, vibration generator, etc.

The system 100b further comprises a controller 108b that is configured to control at least one function of the device 101b. In the illustrated embodiment, the controller 108b is a component of the device 101b, but in other embodiments may be separate from (but connectable to) the device 101b. The controller 108b is configured to control the operation of the heater 104b and, for example, may be configured to control the voltage applied from the power source 105b to the heater 104b. The controller 108b may be configured to toggle the supply of power to the heater 104b between an on state, in which the full output voltage of the power source 105b is applied to the heater 104b, and an off state, in which the no voltage is applied to the heater 104b.

Although not shown, the system 100b may also comprise a voltage regulator to regulate the output voltage from the power source 105b to form a regulated voltage. The regulated voltage may then be applied to the heater 104b.

In addition to being connected to the heater 104b, the controller 108b is operatively connected to the UI 107b. Thus, the controller 108b may receive an input signal from the input means of the UI 107b. Similarly, the controller 108b may transmit output signals to the UI 107b. In response, the output means of the UI 107b may convey information, based on the output signals, to a user. The controller also comprises a memory 109b, which is a non-volatile memory. The memory 109b includes instructions, which, when implemented, cause the controller to perform certain tasks or steps of a method.

FIG. 8B is a schematic showing a variation of the system 100b of FIG. 8A. In the system 100b′ of FIG. 8B, the heater 104b forms part of the device 101b, rather than the consumable 102b. In this variation, the heater 104b is electrically connected to the power source 105b.

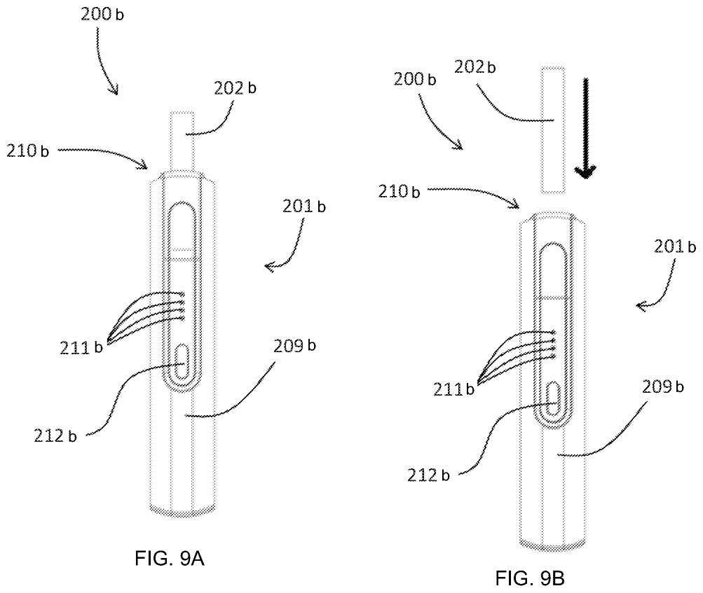

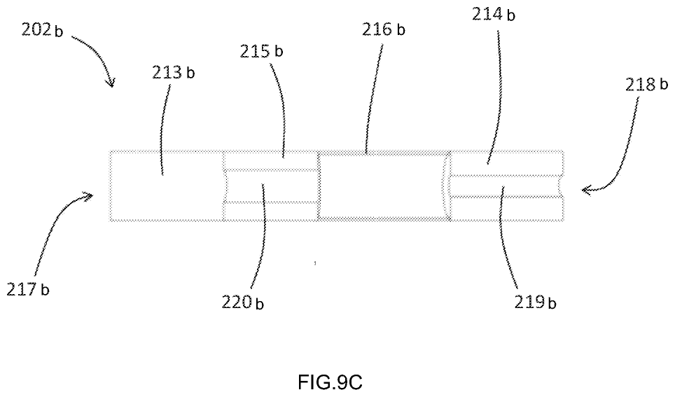

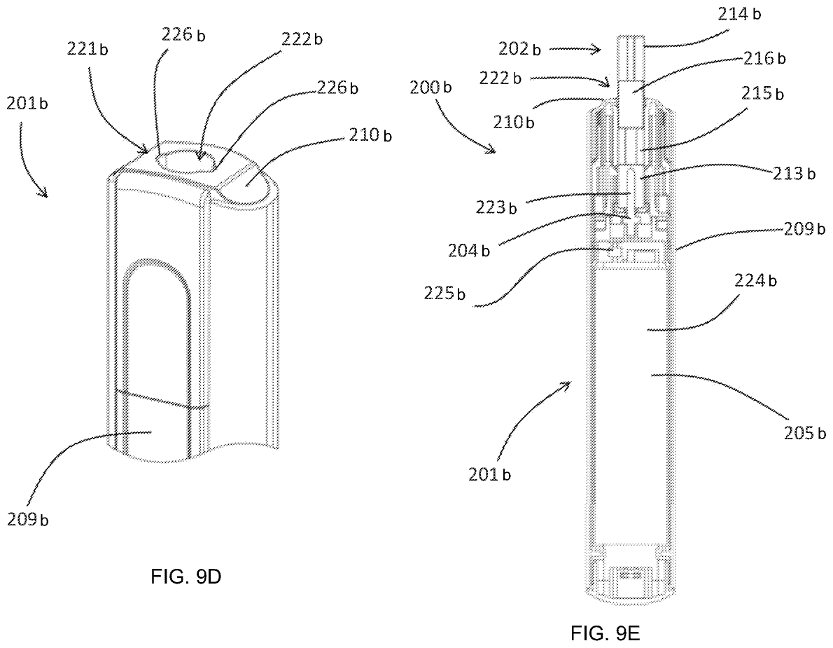

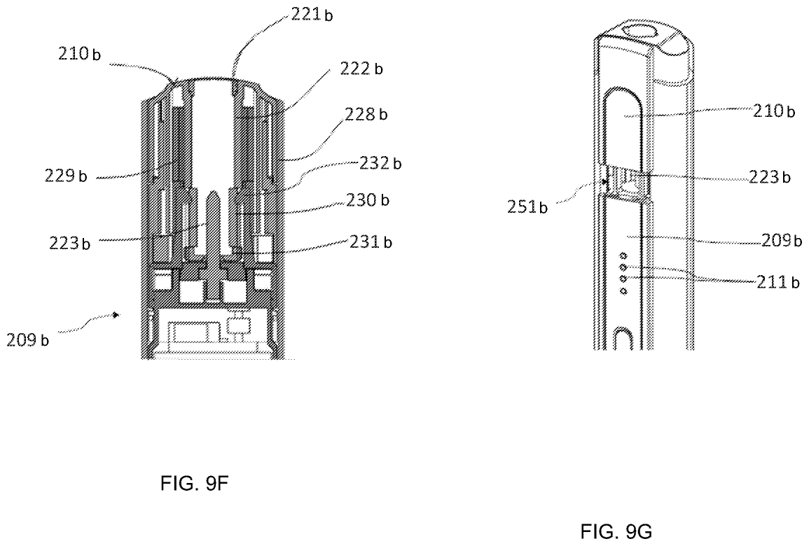

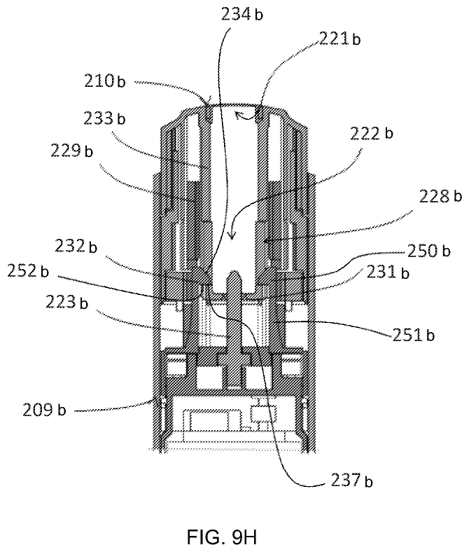

FIG. 9A and FIG. 9B illustrate a heated-tobacco (HT) smoking substitute system 200b. The system 200b is an example of the systems 100b, 100b′ described in relation to FIG. 8A or FIG. 8B. System 200b includes an HT device 201b and an HT consumable 202b. The description of FIG. 8A and FIG. 8B above is applicable to the system 200b of FIG. 9A and FIG. 9B and will thus not be repeated.

The device 201b and the consumable 202b are configured such that the consumable 202b can be engaged with the device 201b. FIG. 9A shows the device 201b and the consumable 202b in an engaged state, whilst FIG. 9B shows the device 201b and the consumable 202b in a disengaged state.

The device 201b comprises a body 209b and cap 210b. In use the cap 210b is engaged at an end of the body 209b. Although not apparent from the figures, the cap 210b is moveable relative to the body 209b. In particular, the cap 210b is slidable and can slide along a longitudinal axis of the body 209b, by a sliding mechanism.

The device 201b comprises an output means (forming part of the UI of the device 201b) in the form of a plurality of light-emitting diodes (LEDs) 211b arranged linearly along the longitudinal axis of the device 201b and on an outer surface of the body 209b of the device 201b. A button 212b is also arranged on an outer surface of the body 209b of the device 201b and is axially spaced (i.e., along the longitudinal axis) from the plurality of LEDs 211b.

FIG. 9C show a detailed section view of the consumable 202b of the system 200b. The consumable 202b generally resembles a cigarette. In that respect, the consumable 202b has a generally cylindrical form with a diameter of 7 mm and an axial length of 70 mm. The consumable 202b comprises an aerosol forming substrate 213b, a terminal filter element 214b, an upstream filter element 215b and a spacer element 216b. In other embodiments, the consumable may further comprise a cooling element. A cooling element may exchange heat with vapor that is formed by the aerosol-forming substrate 213b in order to cool the vapor so as to facilitate condensation of the vapor.

The aerosol-forming substrate 213b is substantially cylindrical and is located at an upstream end 217b of the consumable 202b, and comprises the aerosol former of the system 200b. In that respect, the aerosol forming substrate 213b is configured to be heated by the device 201b to release a vapor. The released vapor is subsequently entrained in an airflow flowing through the aerosol-forming substrate 213b. The airflow is produced by the action of the user drawing on a downstream 218b (i.e., terminal or mouth) end of the consumable 202b.

In the present embodiment, the aerosol forming substrate 213b comprises tobacco material that may, for example, include any suitable parts of the tobacco plant (e.g., leaves, stems, roots, bark, seeds and flowers). The tobacco may comprise one or more of leaf tobacco, stem tobacco, tobacco powder, tobacco dust, tobacco derivatives, expanded tobacco, homogenized tobacco, shredded tobacco, extruded tobacco, cut rag tobacco and/or reconstituted tobacco (e.g., slurry recon or paper recon). For example, the aerosol-forming substrate 213b may comprise a gathered sheet of homogenized (e.g., paper/slurry recon) tobacco or gathered shreds/strips formed from such a sheet.

In order to generate an aerosol, the aerosol forming substrate 213b comprises at least one volatile compound that is intended to be vaporized/aerosolized and that may provide the user with a recreational and/or medicinal effect when inhaled. The aerosol-forming substrate 213b may further comprise one or more additives. For example, such additives may be in the form of humectants (e.g., propylene glycol and/or vegetable glycerin), flavorants, fillers, aqueous/non-aqueous solvents and/or binders.

The terminal filter element 214b is also substantially cylindrical, and is located downstream of the aerosol forming substrate 213b at the downstream end 218b of the consumable 202b. The terminal filter element 214b is in the form of a hollow bore filter element having a bore 219b (e.g., for airflow) formed therethrough. The diameter of the bore 219b is 2 mm. The terminal filter element 214b is formed of a porous (e.g., monoacetate) filter material. As set forth above, the downstream end 218b of the consumable 202b (i.e., where the terminal filter 214b is located) forms a mouthpiece portion of the consumable 202b upon which the user draws. Airflow is drawn from the upstream end 217b, thorough the components of the consumable 202b, and out of the downstream end 218b. The airflow is driven by the user drawing on the downstream end 218b (i.e., the mouthpiece portion) of the consumable 202b.

The upstream filter element 215b is located axially adjacent to the aerosol-forming substrate 213b, between the aerosol-forming substrate 213b and the terminal filter element 214b. Like the terminal filter 214b, the upstream filter element 215b is in the form of a hollow bore filter element, such that it has a bore 220b extending axially therethrough. In this way, the upstream filter 215b may act as an airflow restrictor. The upstream filter element 215b is formed of a porous (e.g., monoacetate) filter material. The bore 220b of the upstream filter element 215b has a larger diameter (3 mm) than the terminal filter element 214b.

The spacer 216b is in the form of a cardboard tube, which defines a cavity or chamber between the upstream filter element 215b and the terminal filter element 214b. The spacer 216b acts to allow both cooling and mixing of the vapor/aerosol from the aerosol-forming substrate 213b. The spacer has an external diameter of 7 mm and an axial length of 14 mm.

Although not apparent from the figure, the aerosol-forming substrate 213b, upstream filter 215b and spacer 216b are circumscribed by a paper wrapping layer. The terminal filter 214b is circumscribed by a tipping layer that also circumscribes a portion of the paper wrapping layer (so as to connect the terminal filter 214b to the remaining components of the consumable 202b). The upstream filter 215b and terminal filter 214b are circumscribed by further wrapping layers in the form of plug wraps.

Returning now to the device 201b, FIG. 9D illustrates a detailed view of the end of the device 201b that is configured to engage with the consumable 202b. The cap 210b of the device 201b includes an opening 221b to an internal cavity 222b (more apparent from FIG. 9D) defined by the cap 210b. The opening 221b and the cavity 222b are formed so as to receive at least a portion of the consumable 202b. During engagement of the consumable 202b with the device 201b, a portion of the consumable 202b is received through the opening 221b and into the cavity 222b. After engagement (see FIG. 9B), the downstream end 218b of the consumable 202b protrudes from the opening 221b and thus also protrudes from the device 201b. The opening 221b includes laterally disposed notches 226b. When a consumable 202b is received in the opening 221b, these notches 226b remain open and could, for example, be used for retaining a cover in order to cover the end of the device 201b.

FIG. 9E shows a cross section through a central longitudinal plane through the device 201b. The device 201b is shown with the consumable 202b engaged therewith.

The device 201b comprises a heater 204b comprising heating element 223b. The heater 204b forms part of the body 209b of the device 201b and is rigidly mounted to the body 209b. In the illustrated embodiment, the heater 204b is a rod heater with a heating element 223b having a circular transverse profile. In other embodiments the heater may be in the form of a blade heater (e.g., heating element with a rectangular transverse profile) or a tube heater (e.g., heating element with a tubular form).

The heating element 223b of the heater 204b projects from an internal base of the cavity 222b along a longitudinal axis towards the opening 221b. As is apparent from the figure, the length (i.e., along the longitudinal axis) of the heating element is less than a depth of the cavity 222b. In this way, the heating element 223b does not protrude from or extend beyond the opening 221b.

When the consumable 202b is received in the cavity 222b (as is shown in FIG. 9E), the heating element 223b penetrates the aerosol-forming substrate 213b of the consumable 202b. In particular, the heating element 223b extends for nearly the entire axial length of the aerosol-forming substrate 213b when inserted therein. Thus, when the heater 204b is activated, heat is transferred radially from an outer circumferential surface the heating element 223b to the aerosol-forming substrate 213b.

The device 201b further comprises an electronics cavity 224b. A power source, in the form of a rechargeable battery 205b (a lithium-ion battery), is located in electronics cavity 224b.

The device 201b includes a connector (i.e., forming part of an IO module of the device 201b) in the form of a USB port 206b. The connector may alternatively be, for example, a micro-USB port or a USB-C port for examples. The USB port 206b may be used to recharge the rechargeable battery 205b.

The device 201b includes a controller (not shown) located in the electronics cavity 224b. The controller comprises a microcontroller mounted on a printed circuit board (PCB). The USB port 206b is also connected to the controller 208b (i.e., connected to the PCB and microcontroller).

The controller 208b is configured to control at least one function of the device 201b. For example, the controller 208b is configured to control the operation of the heater 204b. Such control of the operation of the heater 204b may be accomplished by the controller toggling the electrical connection of the rechargeable battery 205b to the heater 204b. For example, the controller 208b is configured to control the heater 204b in response to a user depressing the button 212b. Depressing the button 212b may cause the controller to allow a voltage (from the rechargeable battery 205b) to be applied to the heater 204b (so as to cause the heating element 223b to be heated).

The controller is also configured to control the LEDs 211b in response to (e.g., a detected) a condition of the device 201b or the consumable 202b. For example, the controller may control the LEDs to indicate whether the device 201b is in an on state or an off state (e.g., one or more of the LEDs may be illuminated by the controller when the device is in an on state).

The device 201b comprises a further input means (i.e., in addition to the button 212b) in the form of a puff sensor 225b. The puff sensor 225b is configured to detect a user drawing (i.e., inhaling) at the downstream end 218b of the consumable 202b. The puff sensor 225b may, for example, be in the form of a pressure sensor, flowmeter or a microphone. The puff sensor 225b is operatively connected to the controller 208b in the electronics cavity 224b, such that a signal from the puff sensor 225b, indicative of a puff state (i.e., drawing or not drawing), forms an input to the controller 208b (and can thus be responded to by the controller 208b).

FIG. 9F illustrates a cross-section through a central longitudinal plane of the device 201b, with the cap 210b engaged with the body 209b. In the illustrated embodiment, the body 209b of the device 201b (seen in FIG. 9B), includes first and second guideways 228b, 229b, which facilitate movement or displacement of the cap 210b between a first position in which the cap is fully engaged with the body (as seen in FIG. 9F) and a second position in which the cap is longitudinally displaced from the body. A plurality of first guideways 228b may be defined on an inner circumference of the body 209b, at the interface of the body 209b and the cap 210b. The plurality of first guideways 228b are configured to receive the cap 201b and allow movement of the cap 210b relative to the body 209b, so as to accommodate the cap 210b or a portion of the cap 210b in the body 209b. Further, a plurality of second guideways 229b are also defined in the body 209b, about the heating element 223b. The plurality of second guideways 229b are configured to receive the cap 210b such that the cavity 222b defined by the cap 210b is circumscribed by the plurality of second guideways 229b, during engagement of the cap 210b with the body 209b. The cavity 222b of the cap 210b is configured to traverse on the plurality of second guideways 229b to circumscribe the heating element 223b, upon engagement of the cap 210b with the body 209b.

In some embodiments, the cap 210b may be displaced relative to the body 209b, by a threaded mechanism.

In some embodiments, the cap 210b and the body 209b are engaged together (i.e., in a close fit) by at least one connecting mechanism, for example a snap fit connection, a magnetic connection and the like, which facilitate in retaining the cap 210b in the first position.

The body 209b further comprises a retainer mechanism (e.g., a detent mechanism), for retaining the cap 210b in the second position relative to the body 209b. The retainer mechanism includes two flexure bearings 232b facing each other. The two flexure bearings 232b are located proximal to the heating element 223b. The two flexure bearings 232b are adapted to engage with an external surface of a walls of the cavity 222b, when the cap 210b is received by the body 209b.

As apparent from the FIG. 9F, when the cap 210b is in the first position (i.e., when the cap 210b is in engagement with the body 209b), the flexure bearings 232b are configured to abut the deformable region 230b of the cavity 222b. The deformable region 230b, deforms due to the force applied by the flexure bearing 232b. The flexure bearing 232b is adapted to deform the deformable region 230b of the cavity 222b towards the heating element 223b of the heater 204b. The deformable region 230b is configured to selectively deform in response to movement of the cap 210b about the flexure bearing 232b.

In an illustrative embodiment as seen in FIG. 9G and FIG. 9H, movement or displacement of the cap 210b from the first position to the second position (e.g., upward movement of the cap 210b as seen in the Figures) is carried out by applying a pull force or an upward force on the cap 210b along a longitudinal axis of the device 201b. During movement of the cap 210b from the first position, the flexure bearings 232b are configured to restrain movement of the cap 210b from the body 209b, as apparent from FIG. 9G beyond the second position (i.e., the lifted condition of the cap 210b from the body 209b). The flexure bearings 232b are configured to engage with the rigid base region 231b of the cavity 222b defined in the cap 210b. Thus, movement of the cap 210b beyond the second position is restricted. That is, the cap 210b is allowed to be lifted to a height (e.g., distance along longitudinal axis of the device 201b) defined between the first position and the second position. As an example, the displacement of the cap between the first position and the second position may range from about 2 mm to about 15 mm, and preferably may be about 5 mm to 8 mm. In a preferred embodiment, the displacement of the cap between the first position and the second position may be about 7 mm. This movement of the cap 210b from the first position to the second position facilitates lifting the consumable 202b away from the heating element along a longitudinal axis. This movement of the cap 210b from the first position to the second position provides an aperture 251b through which the heating element may be inspected/cleaned. Further, the cap 210b is retained in the second position by at least one of the detent mechanism and the magnetic mechanism, which facilitates in maintaining the aperture 251b, to access the heating element 223b.

A portion (e.g., outer surface) of the cap 210b is configured with a tactile (high friction) finish, to facilitate gripping during movement or displacement of the cap 210b between the first position and the second position.

The movement of the cap 210b between the first position and the second position eliminates the need to completely disengage the cap 210b from the body 209b, to access the heating element 223b, which may require periodic cleaning for removing debris accumulated on the heating element 223b, for effective dissipation of heat for generating aerosol.

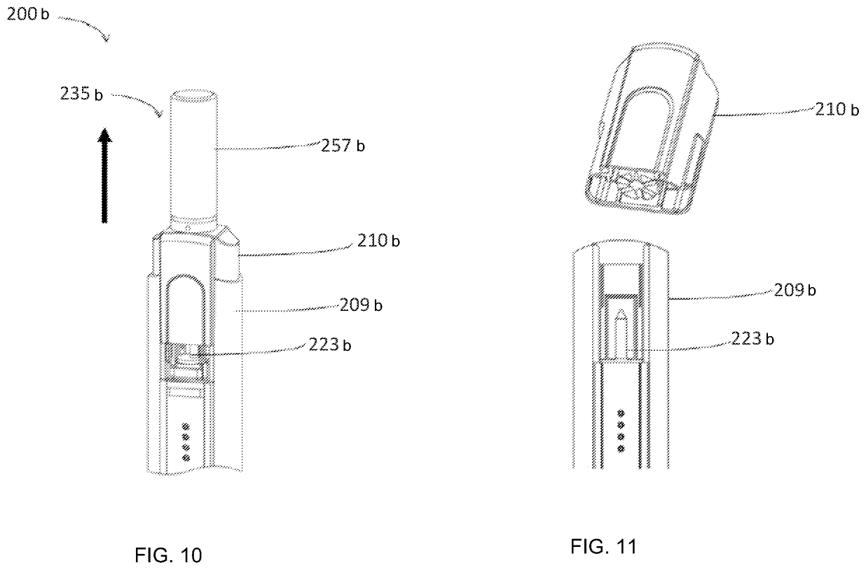

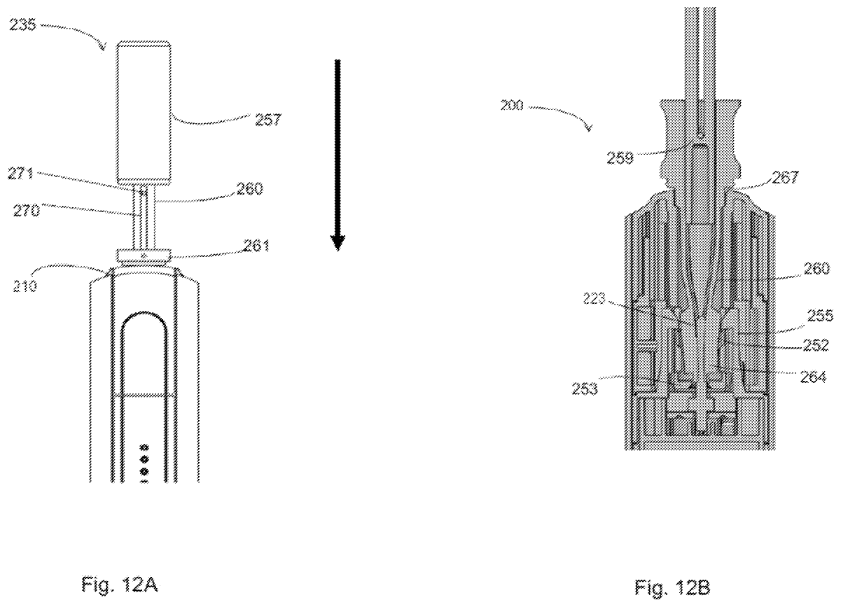

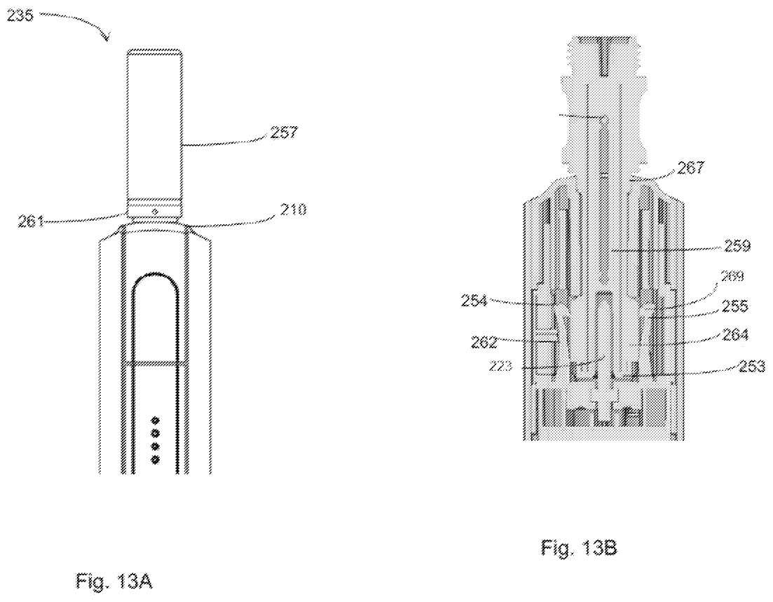

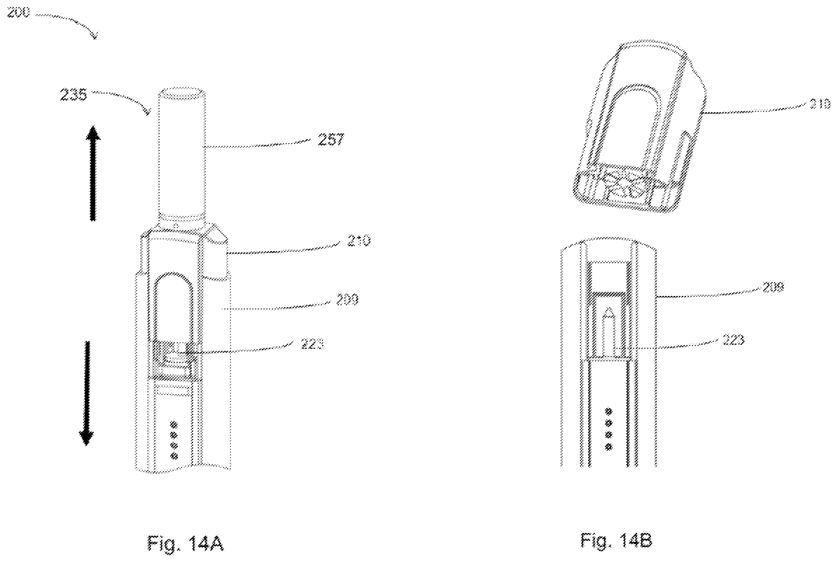

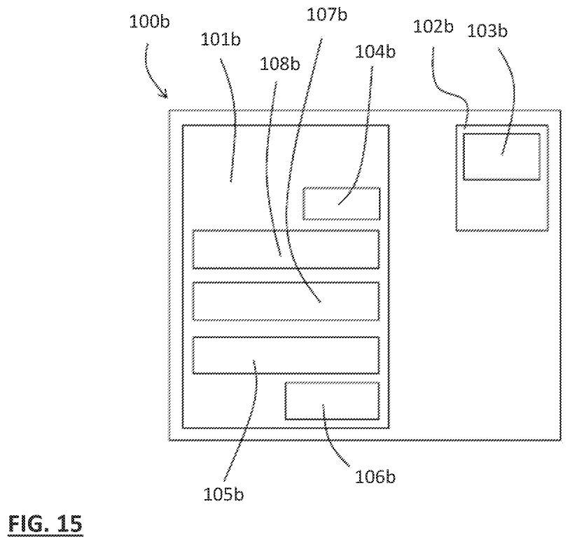

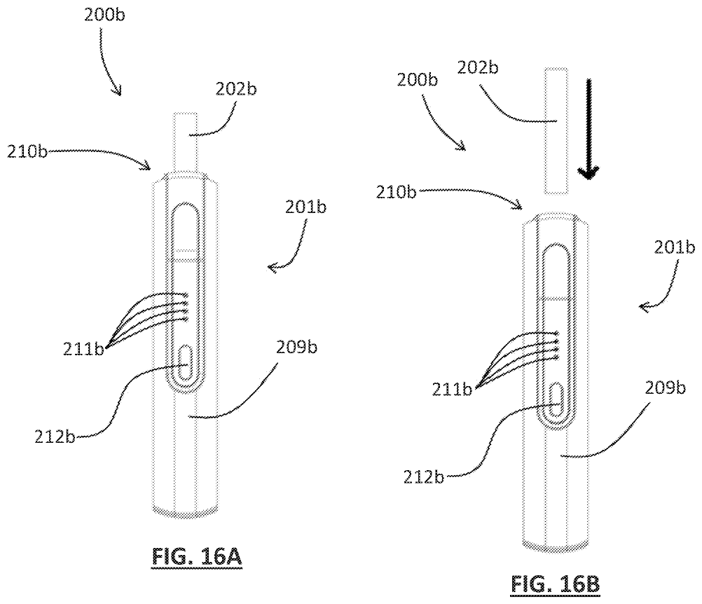

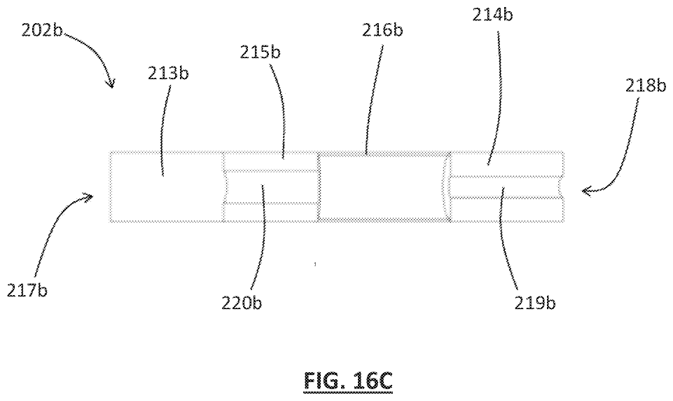

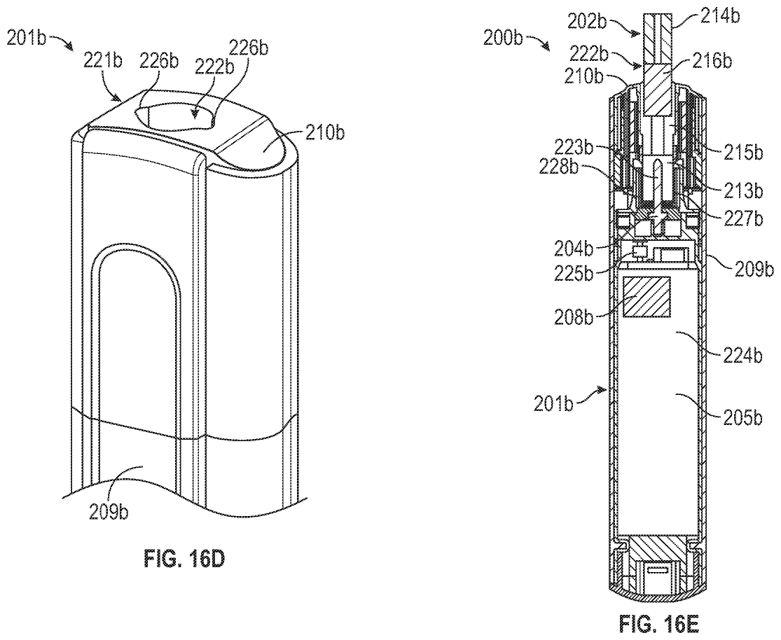

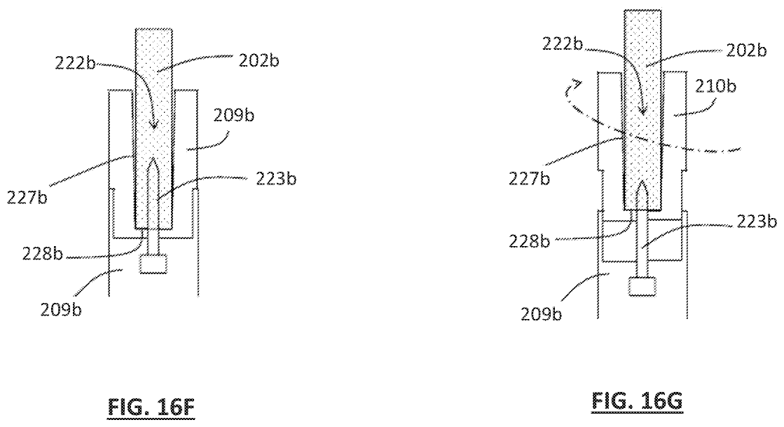

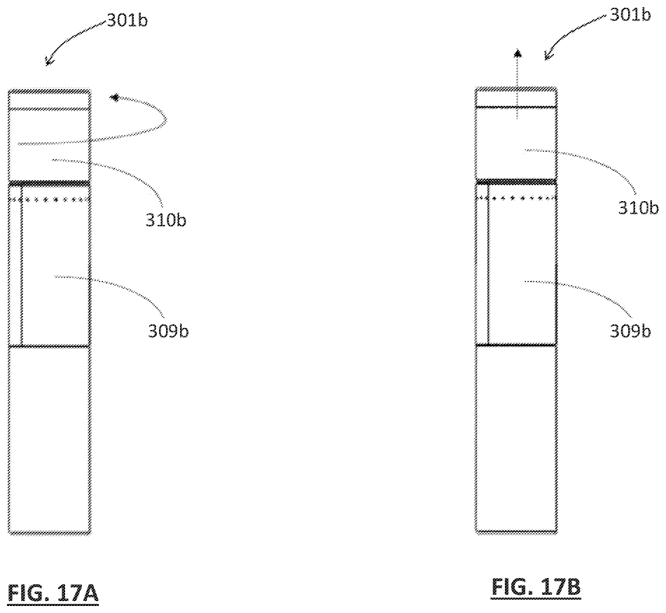

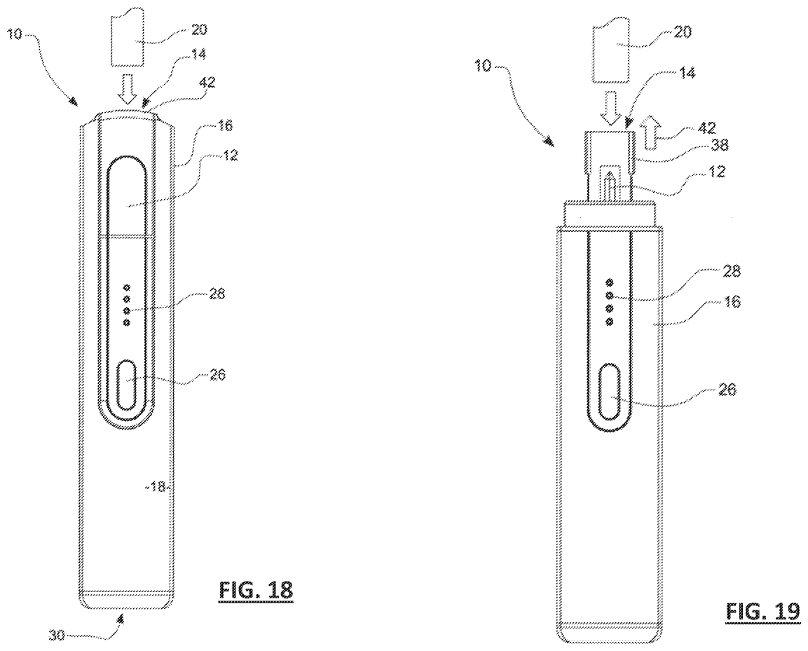

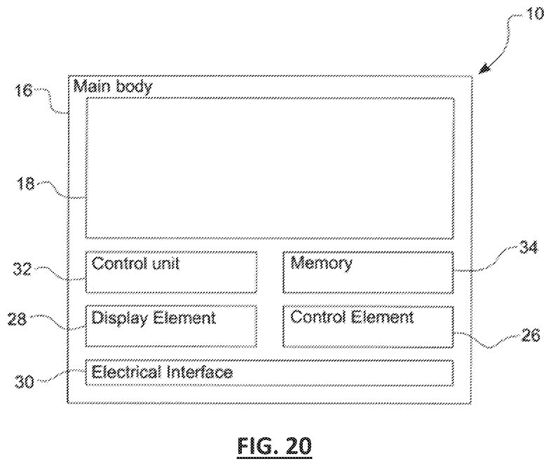

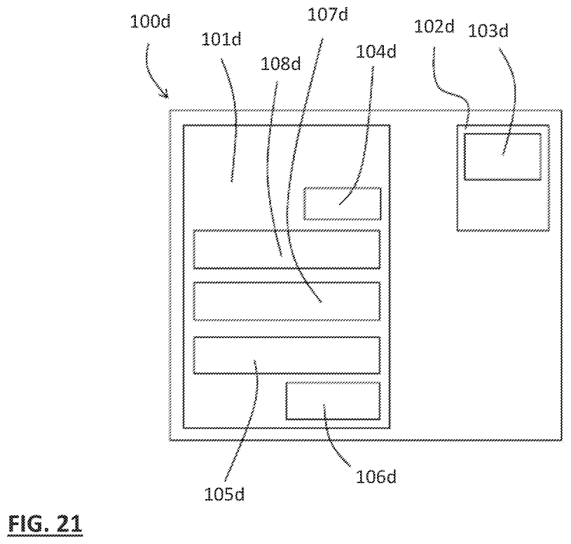

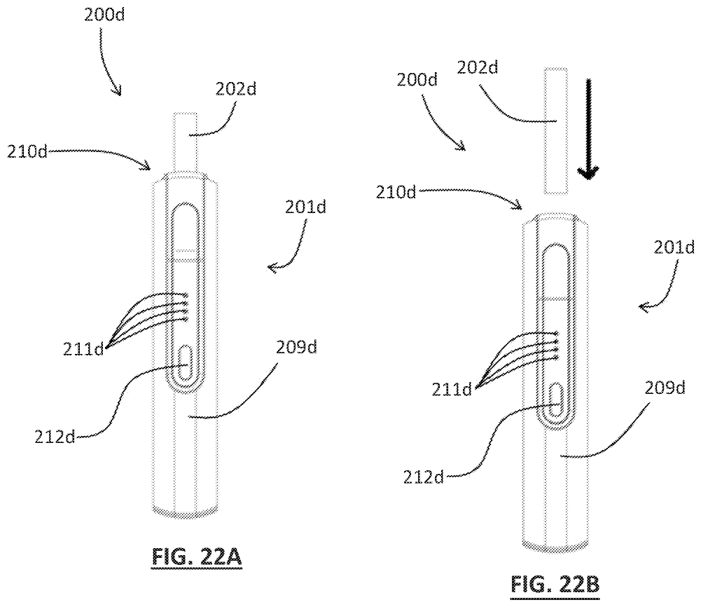

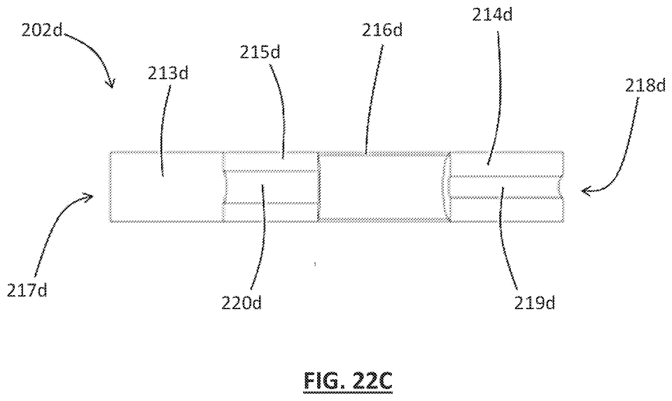

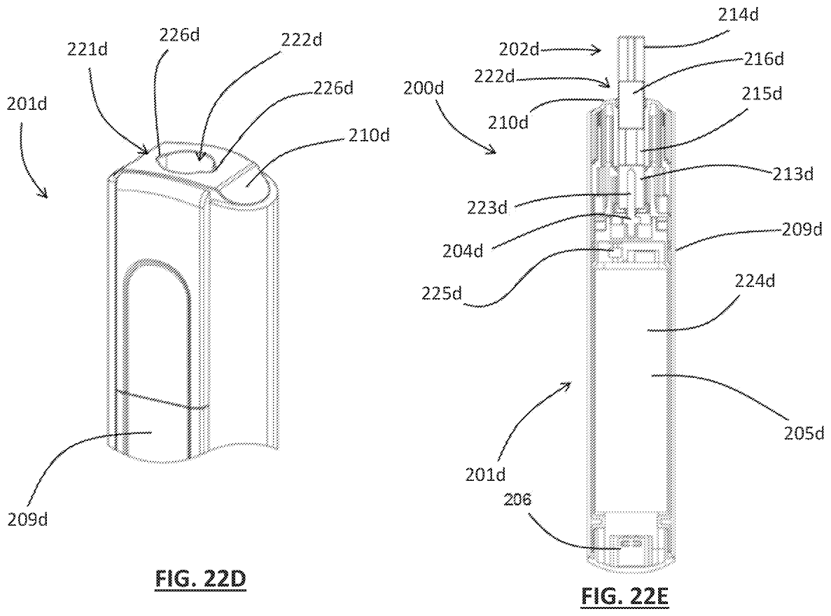

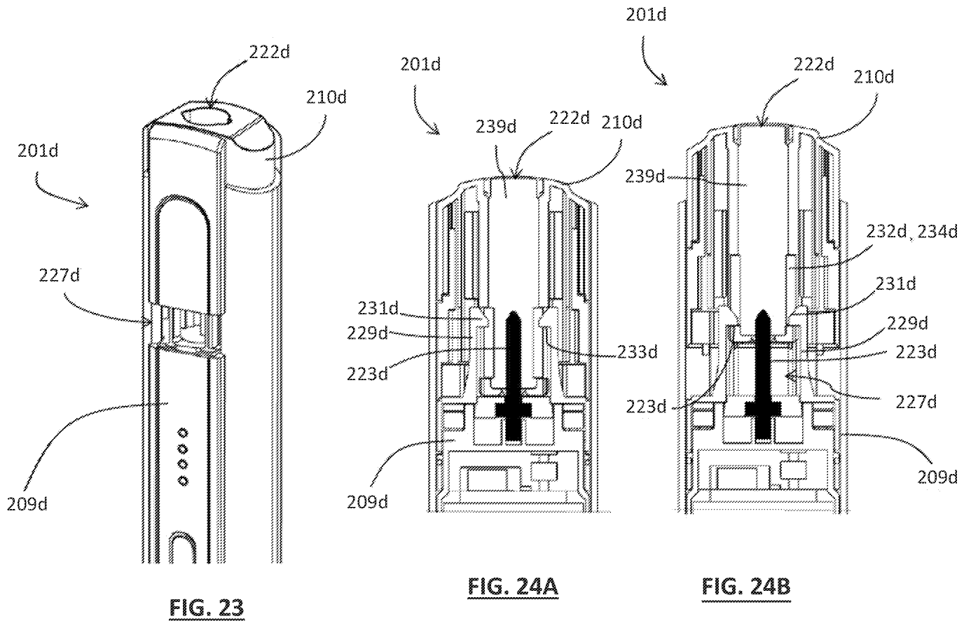

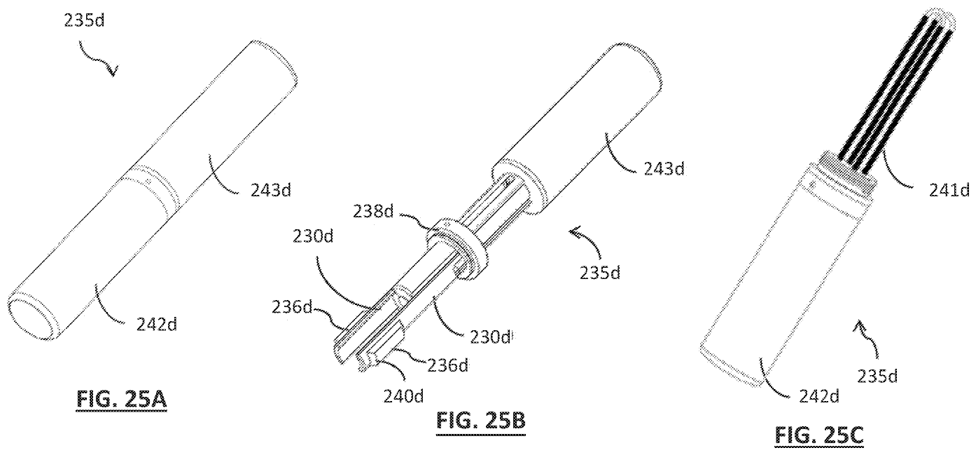

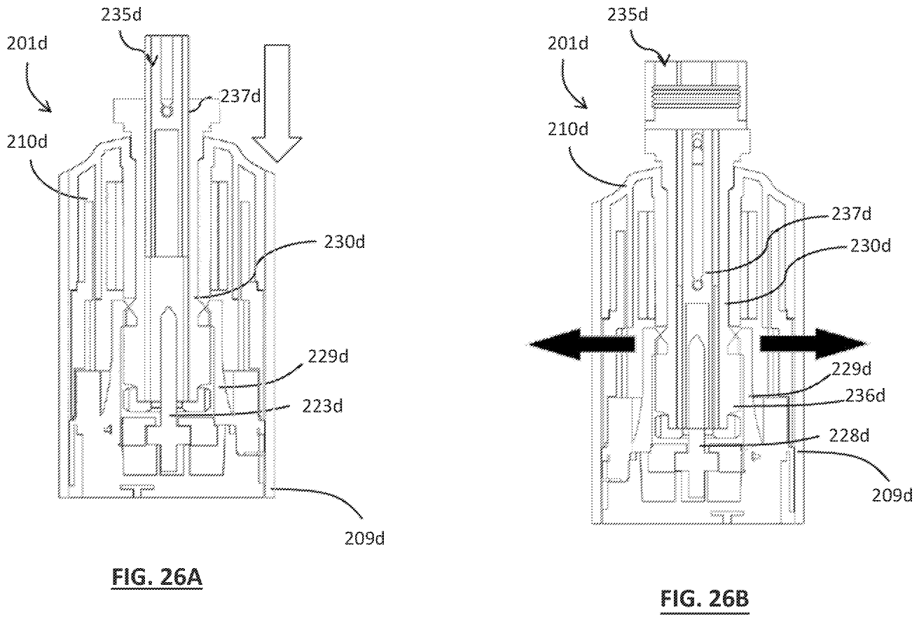

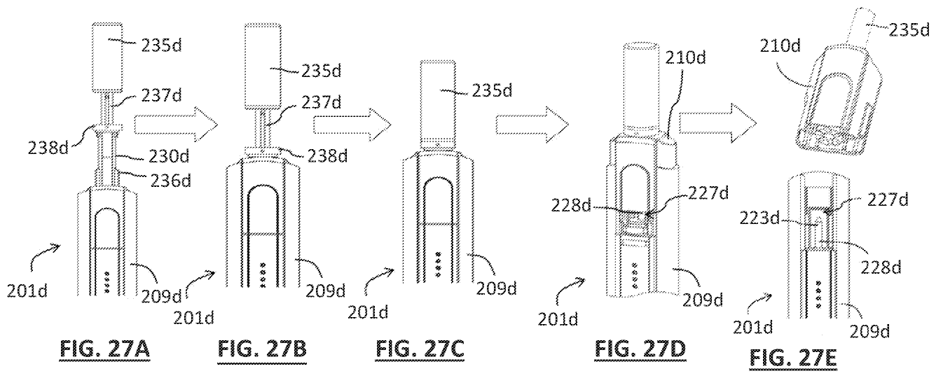

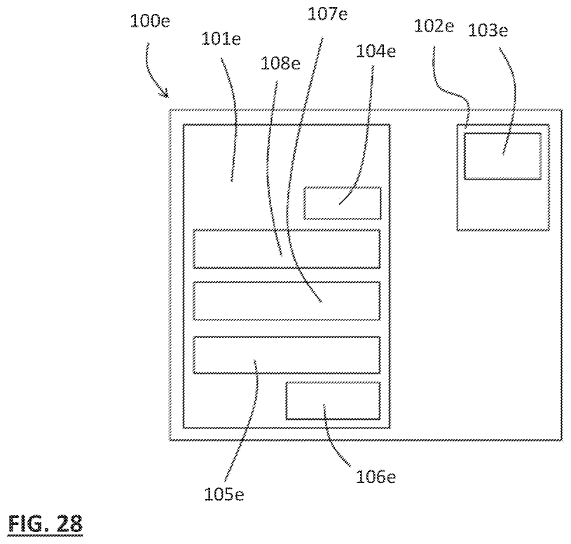

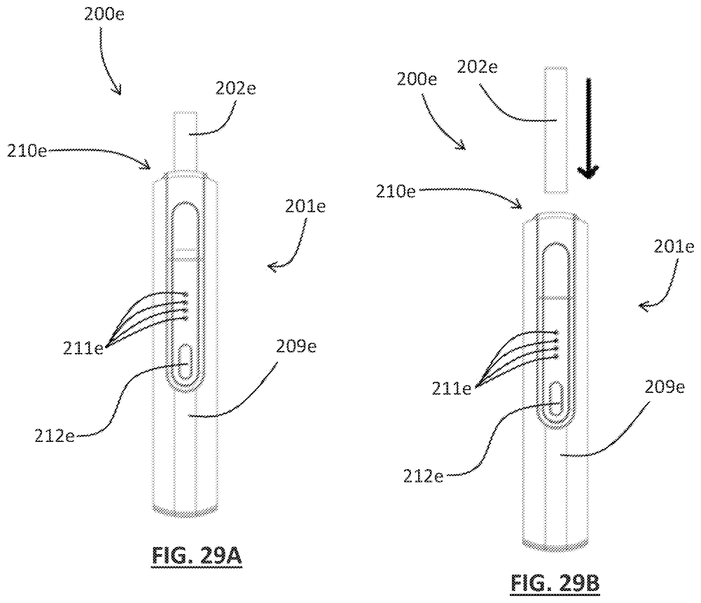

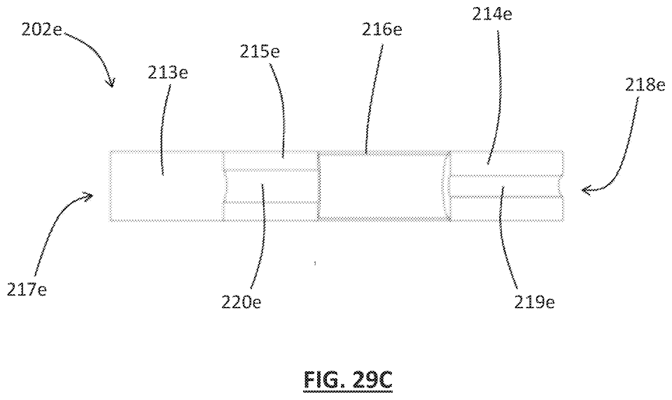

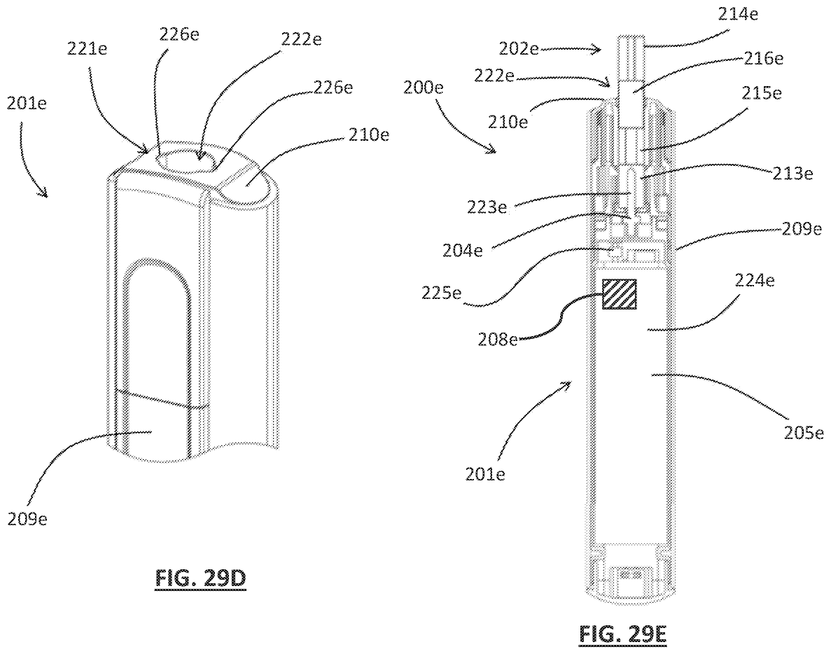

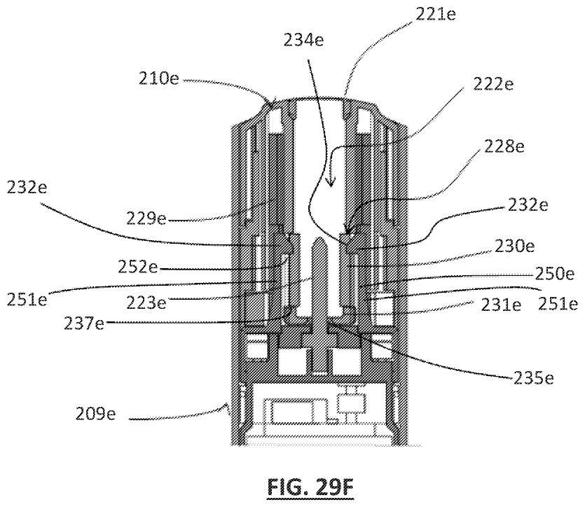

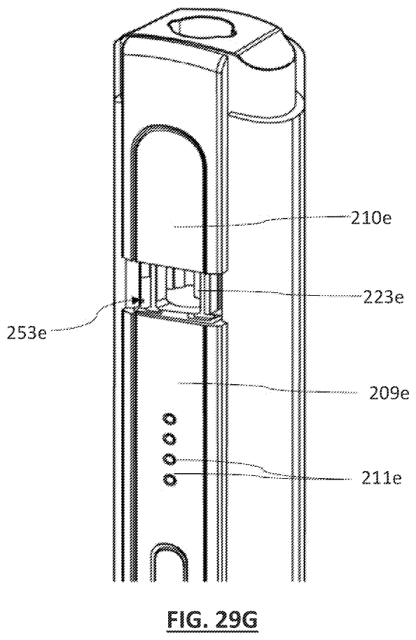

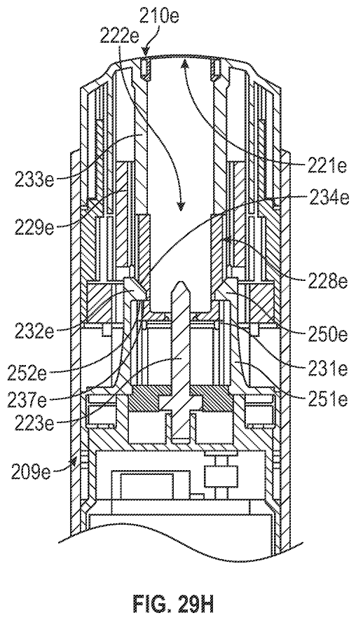

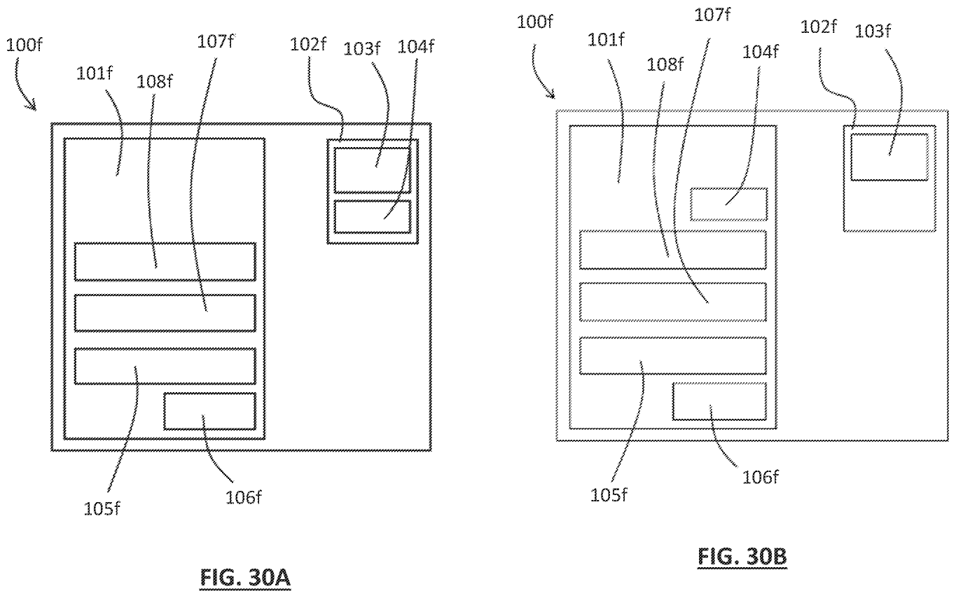

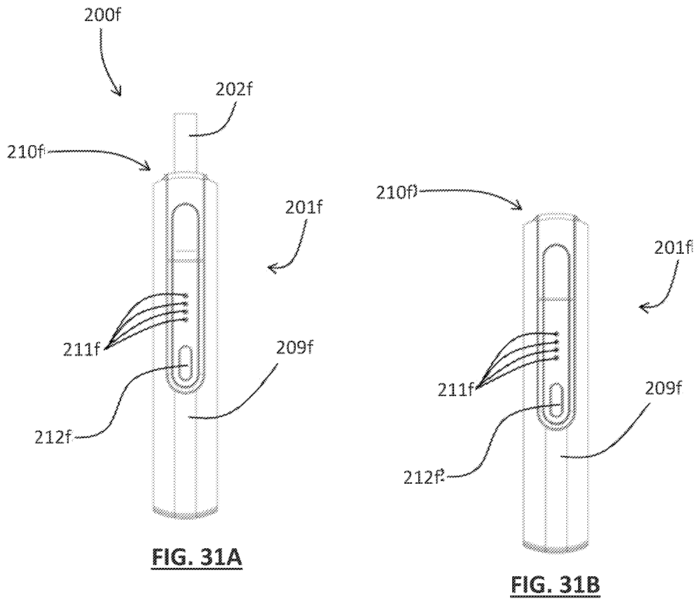

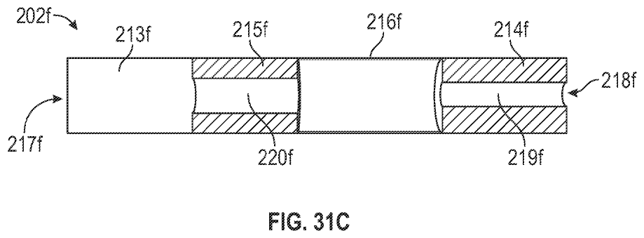

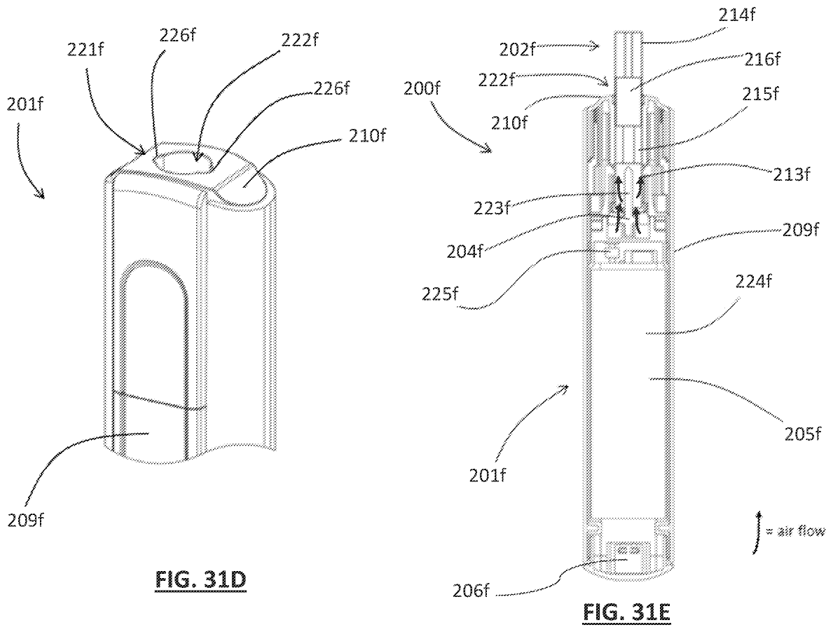

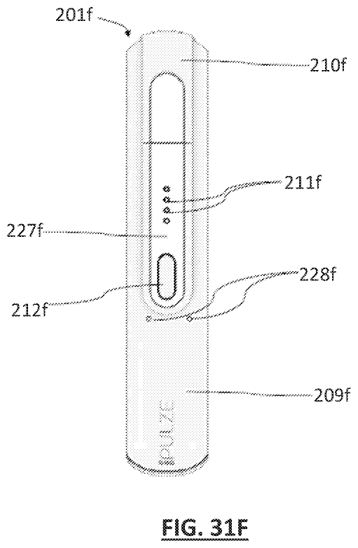

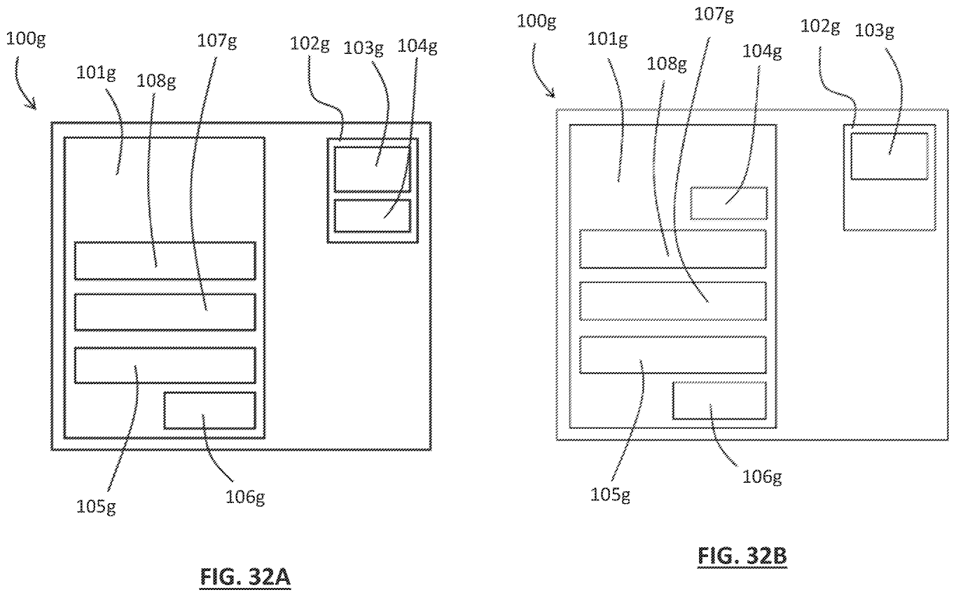

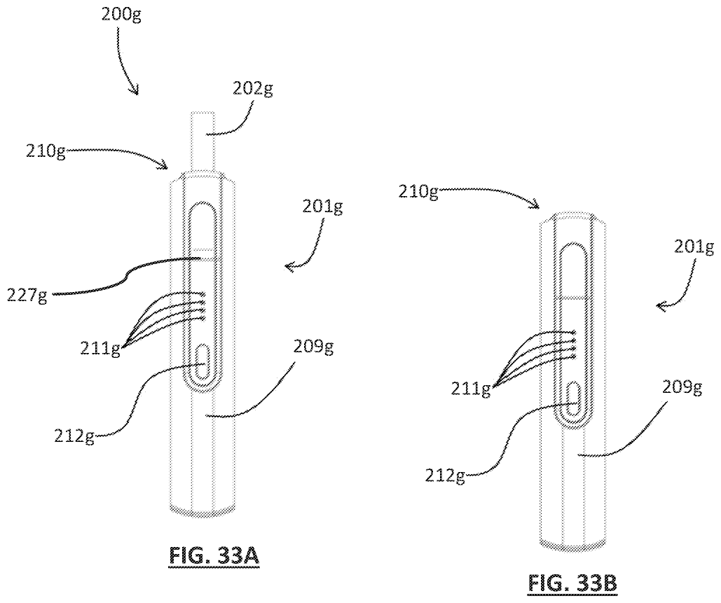

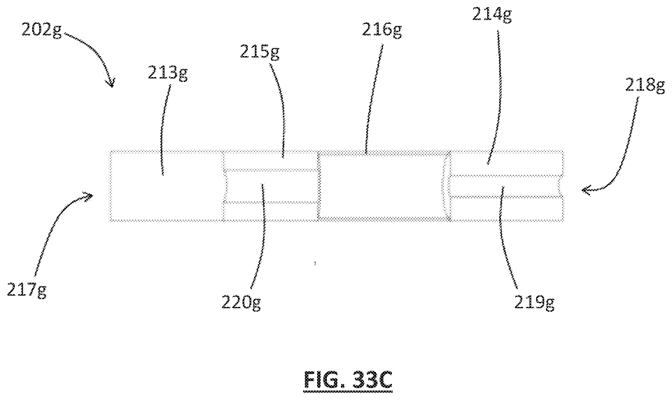

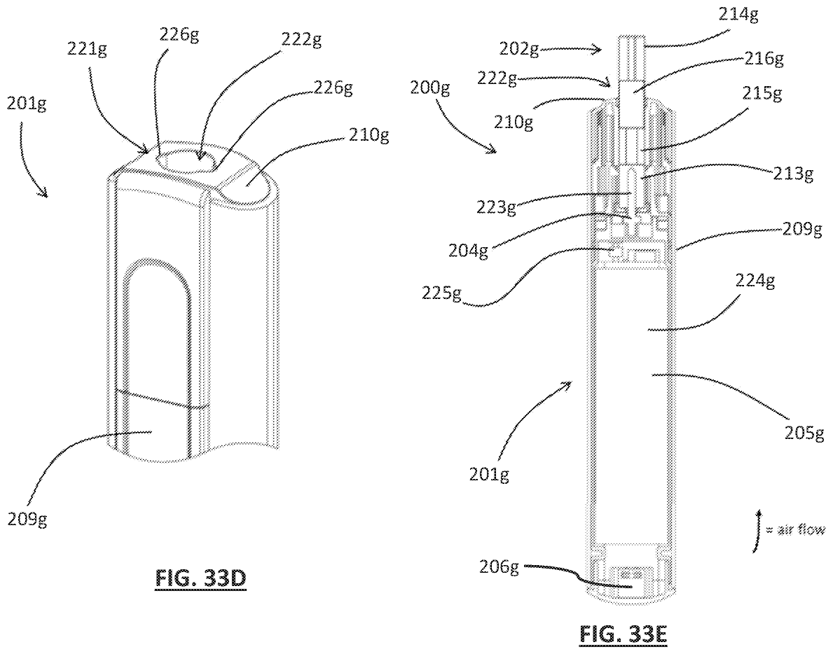

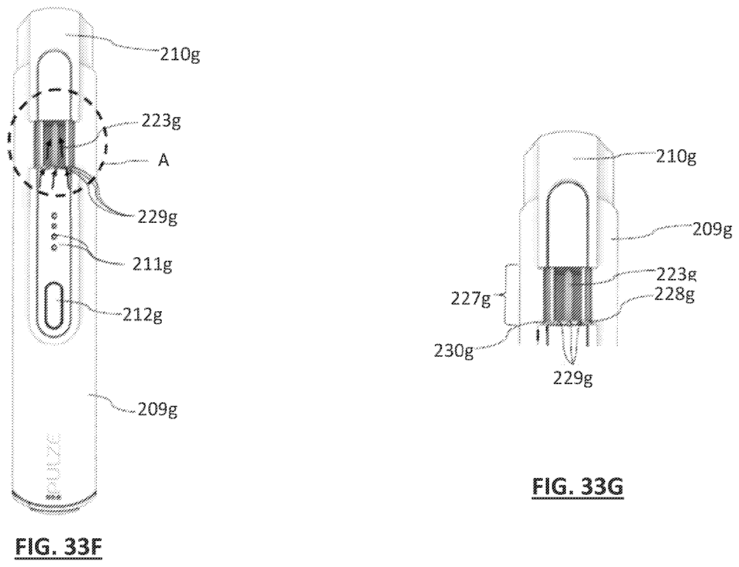

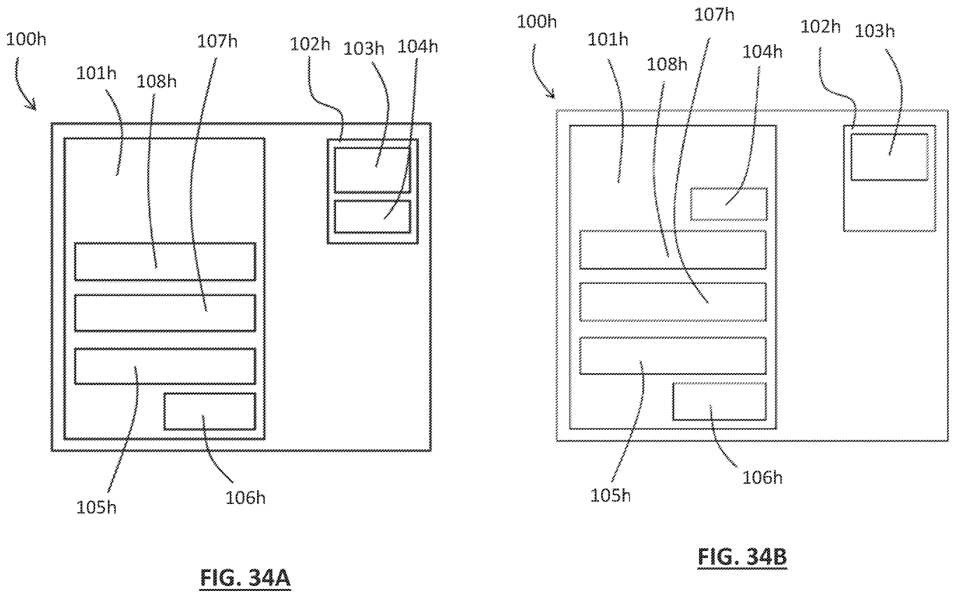

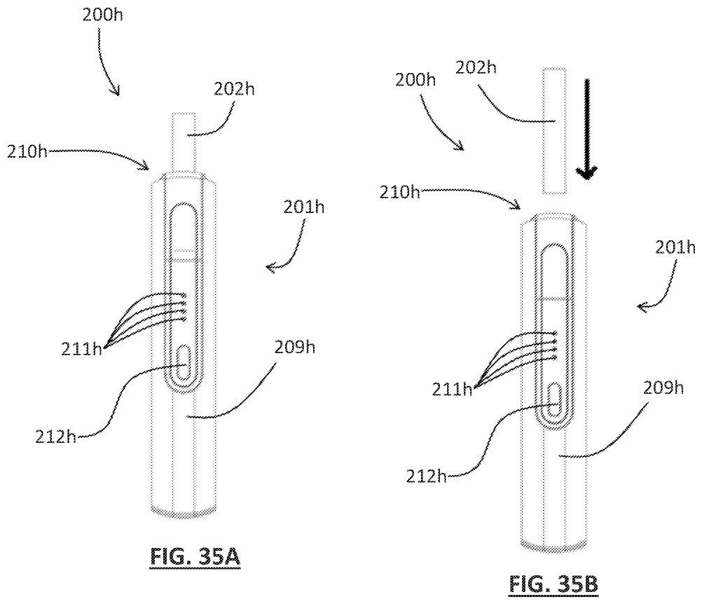

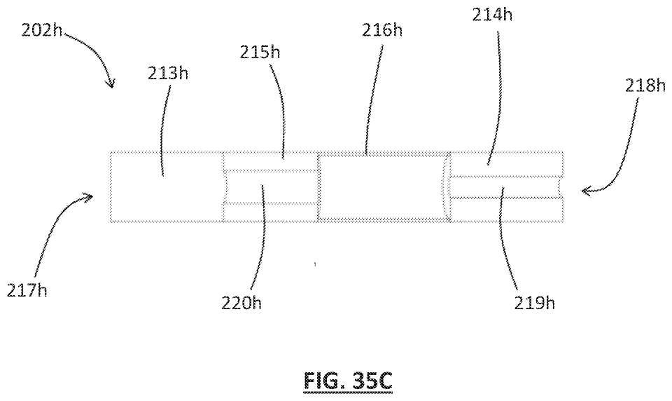

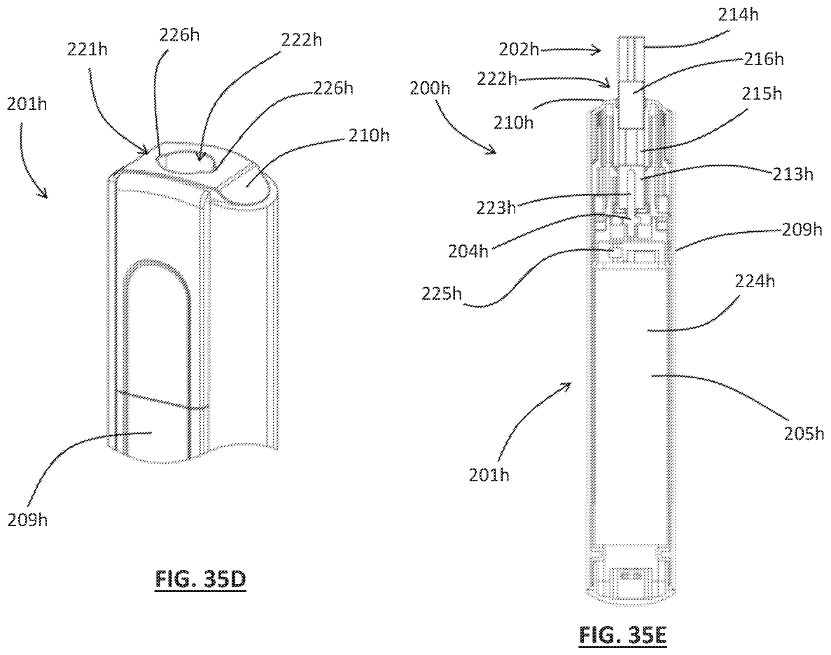

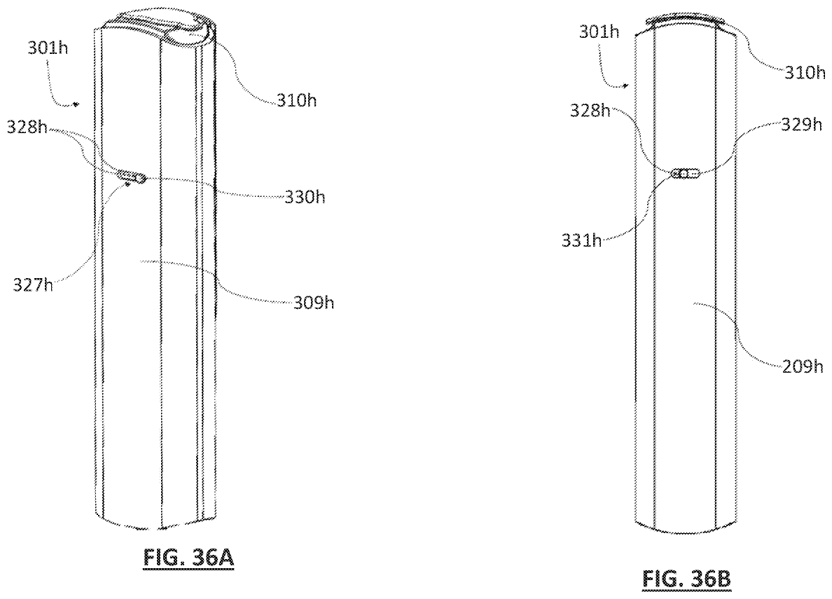

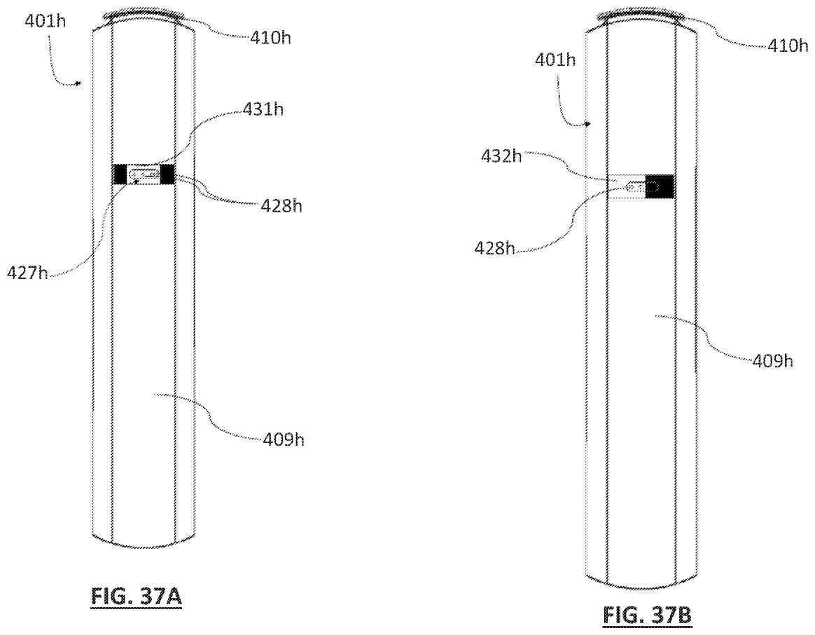

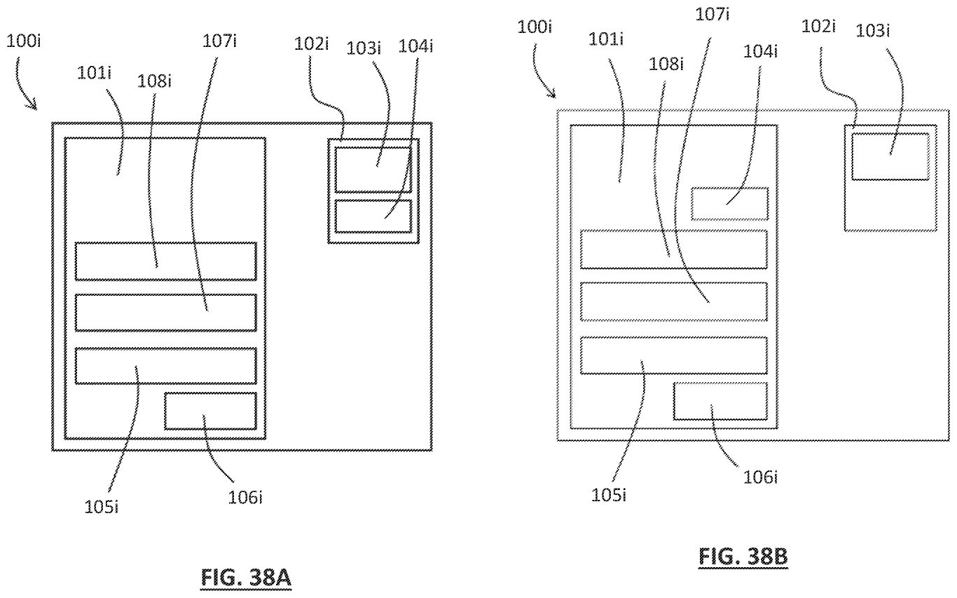

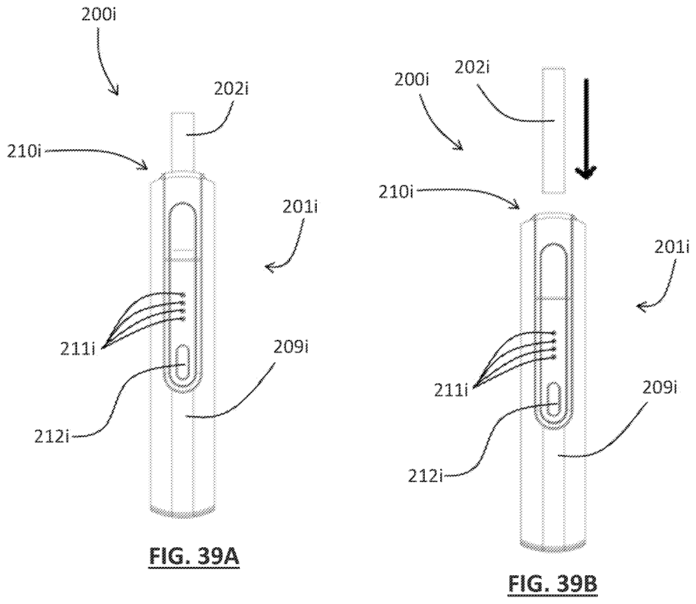

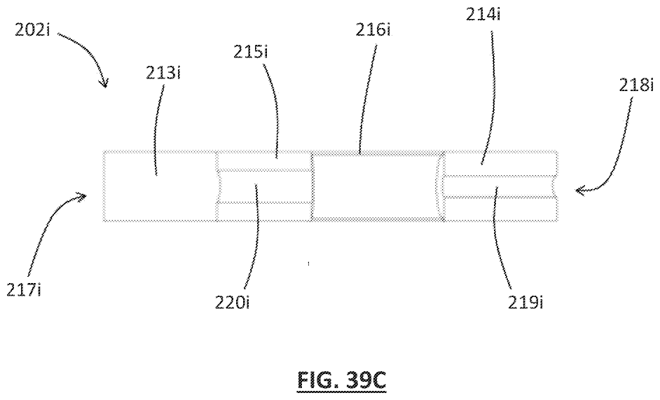

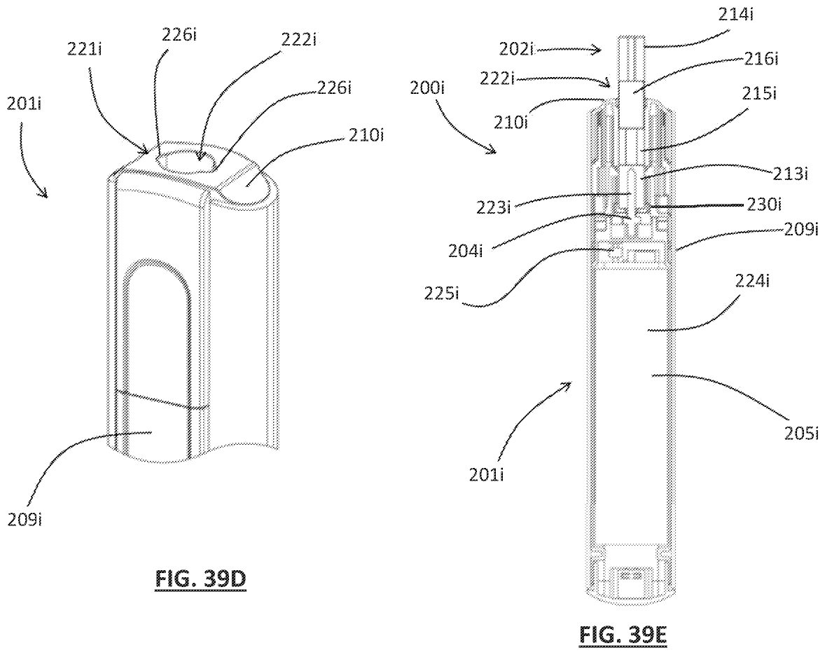

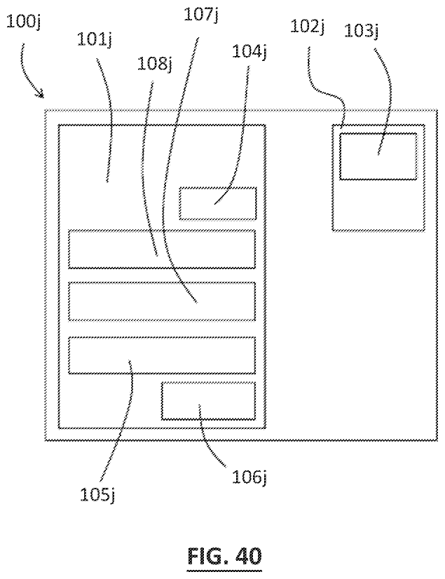

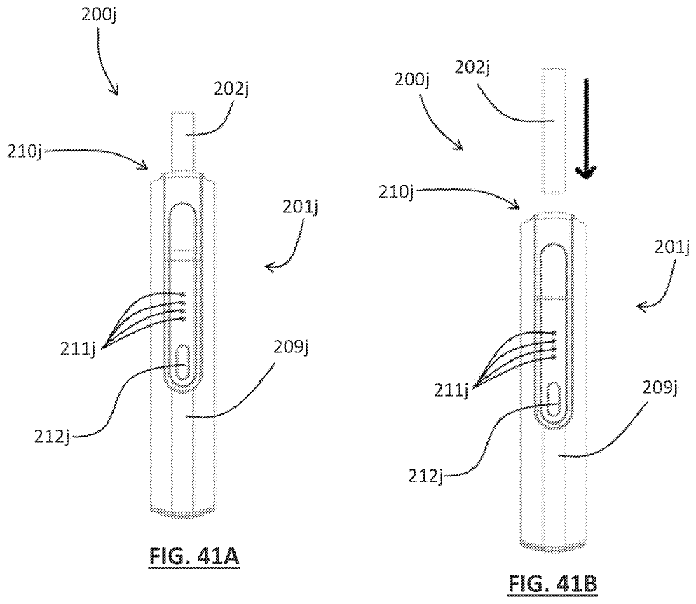

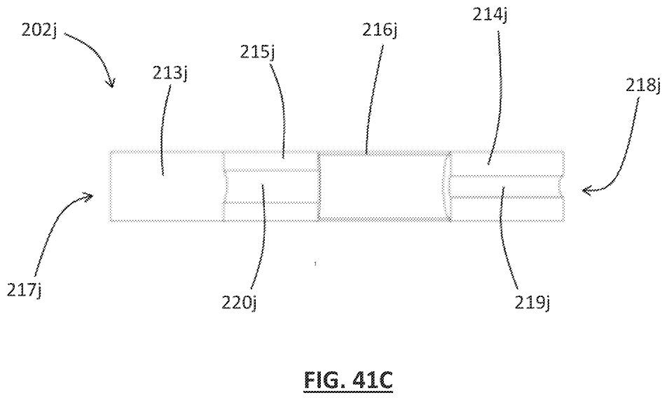

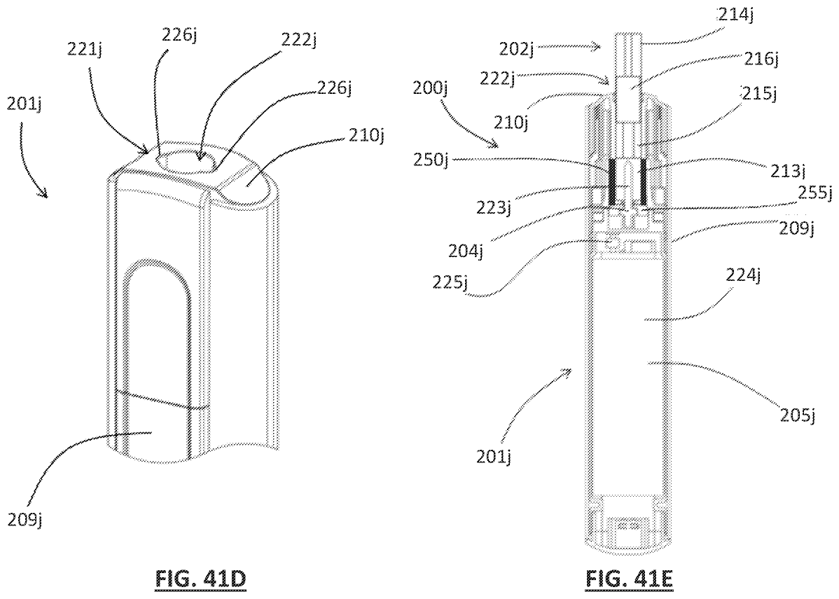

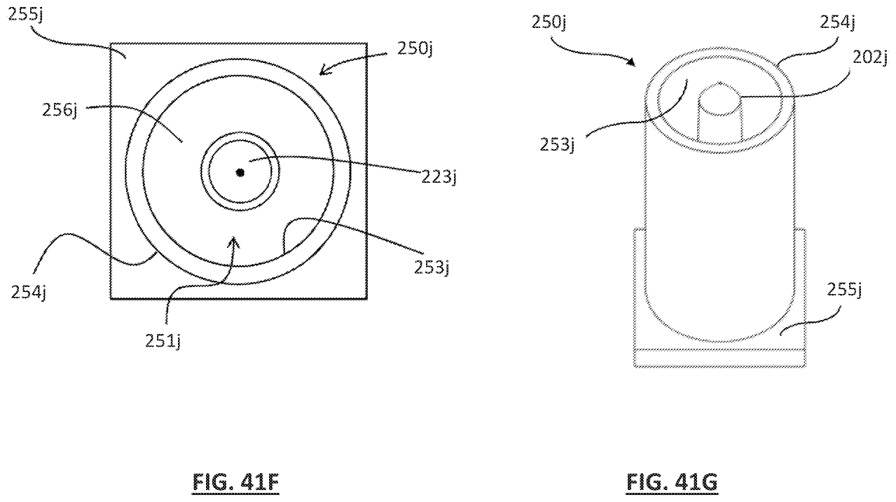

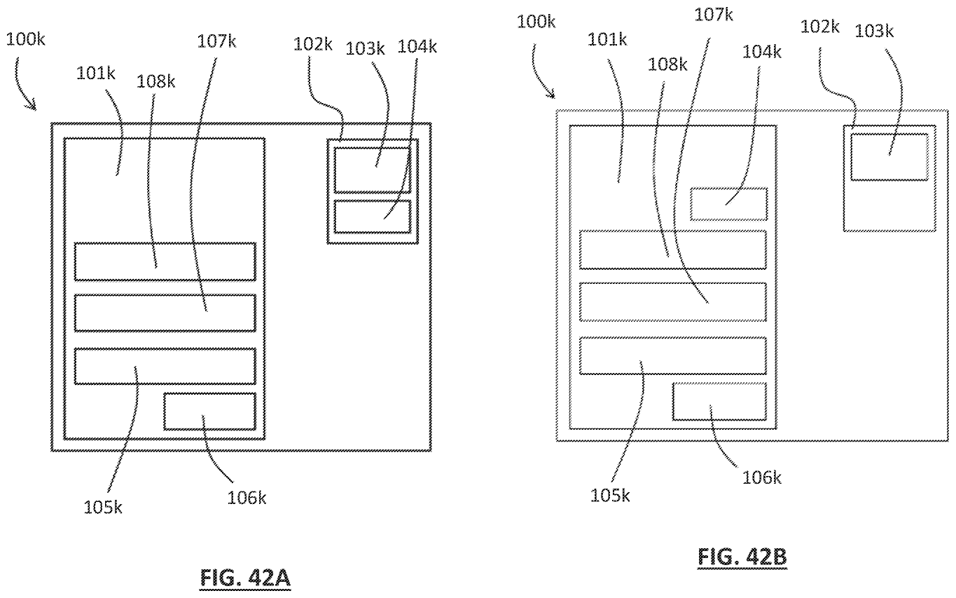

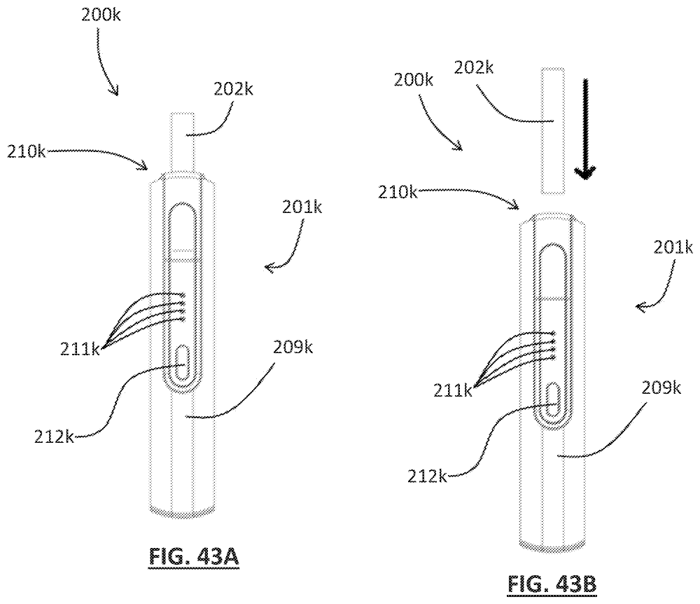

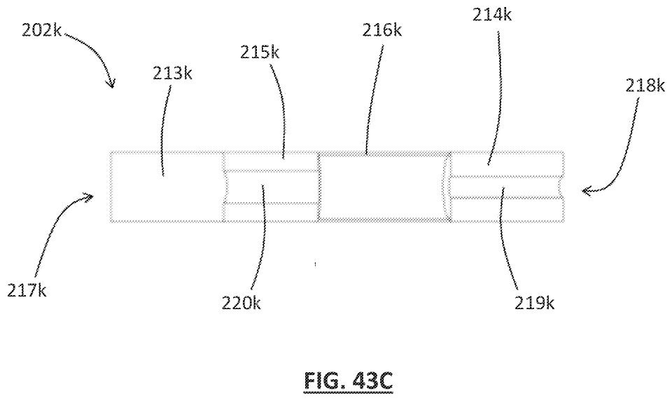

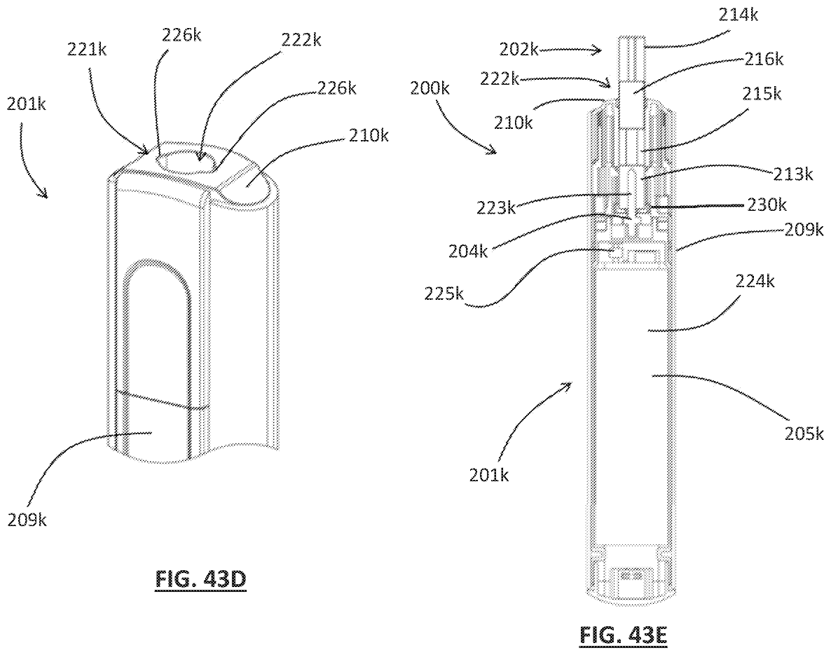

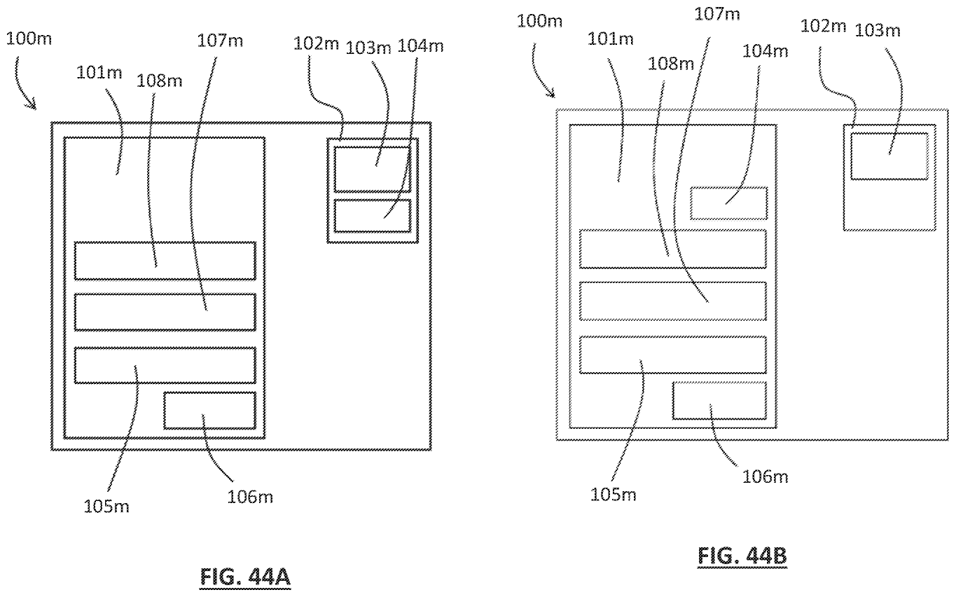

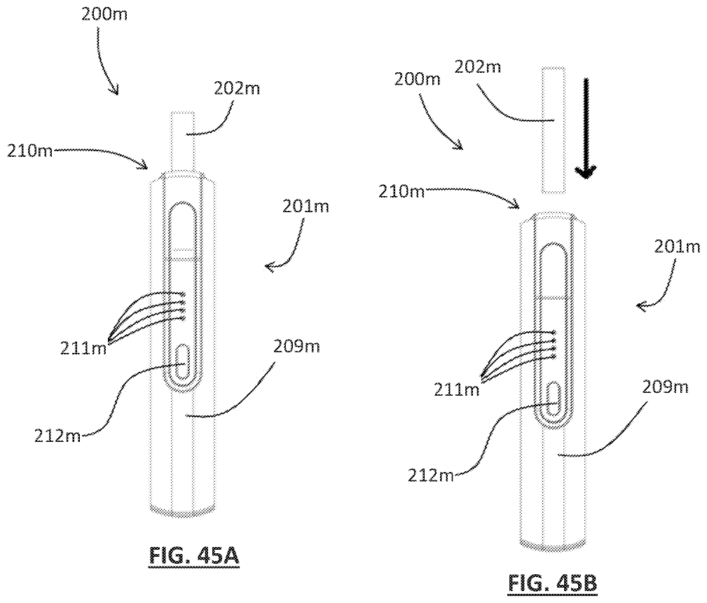

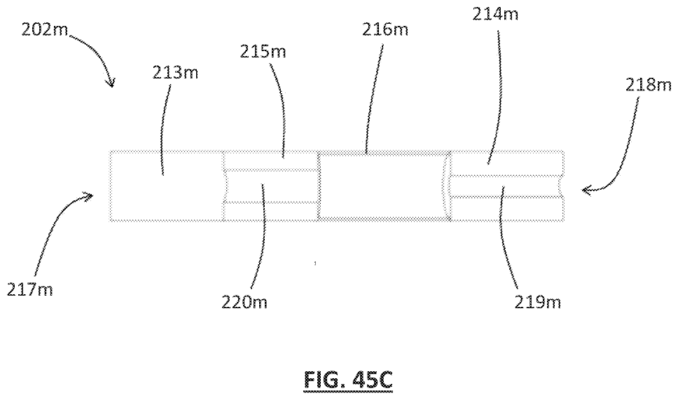

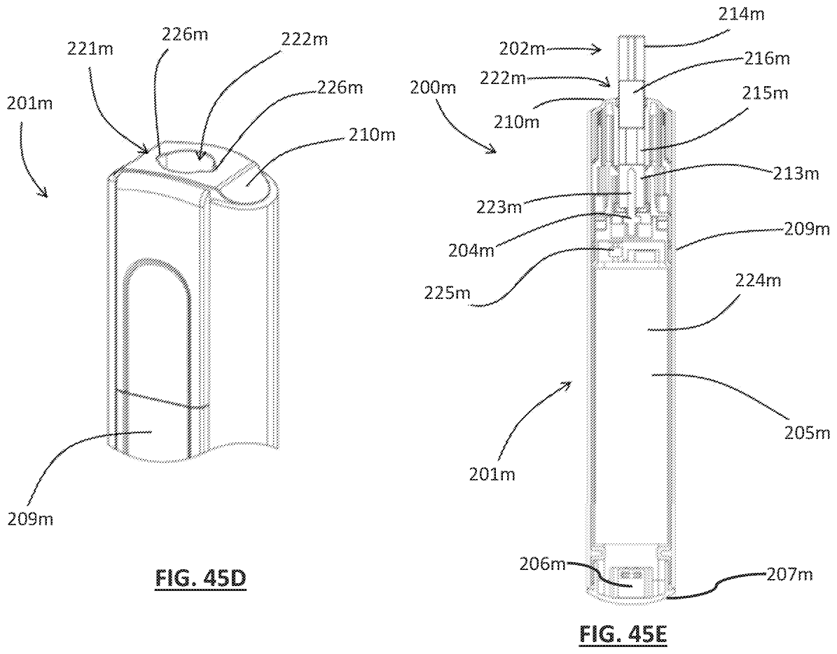

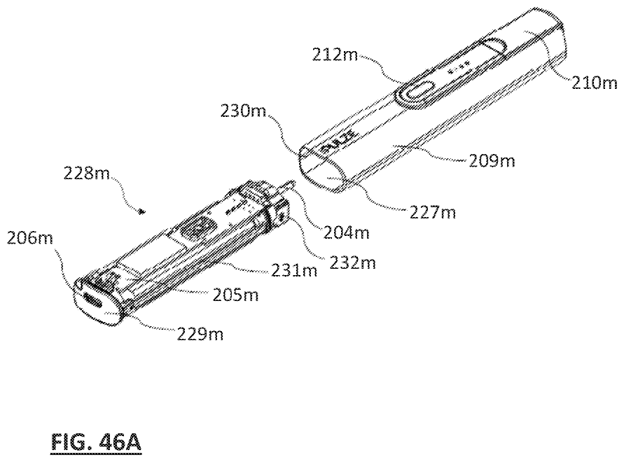

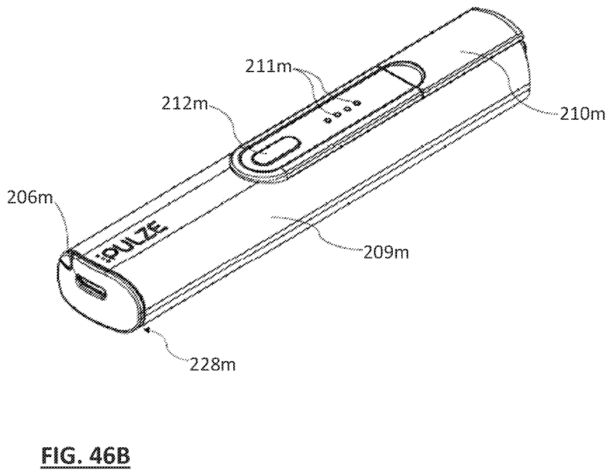

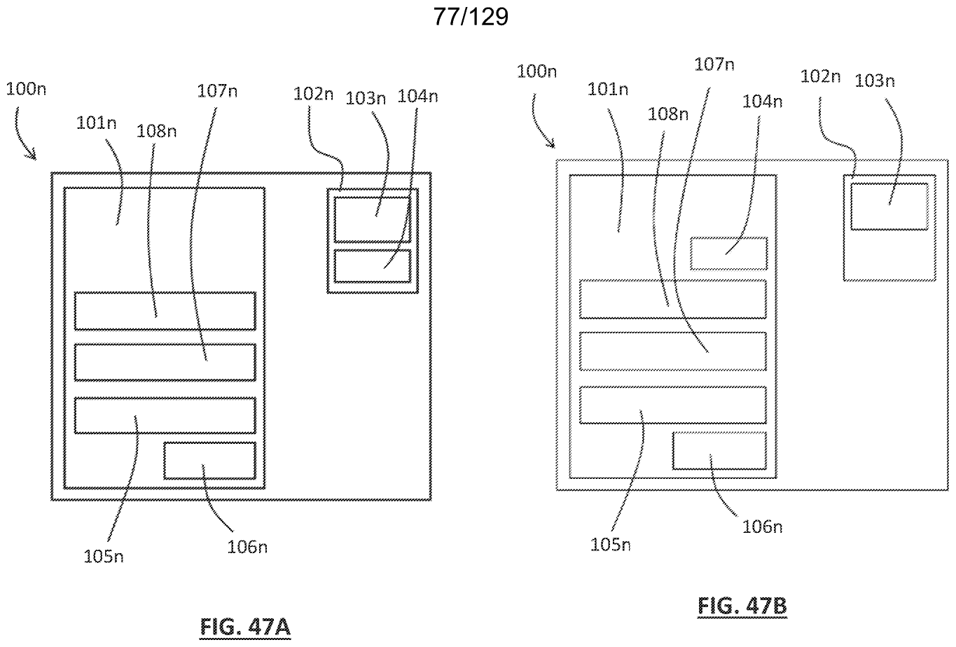

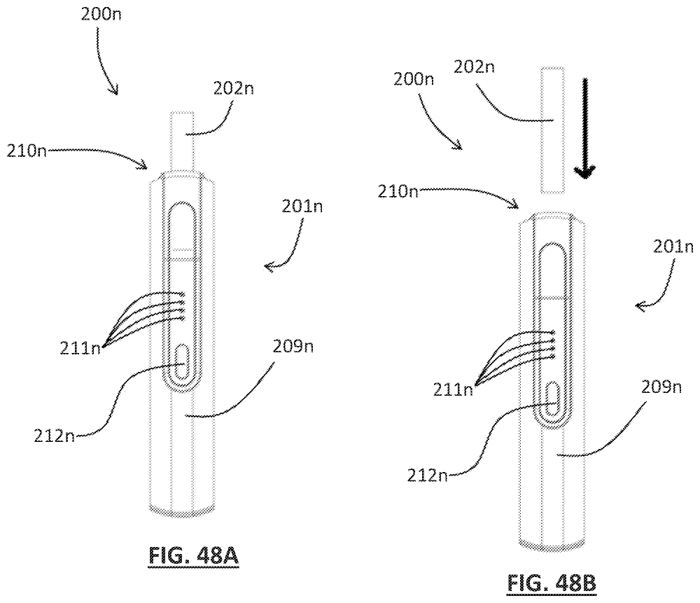

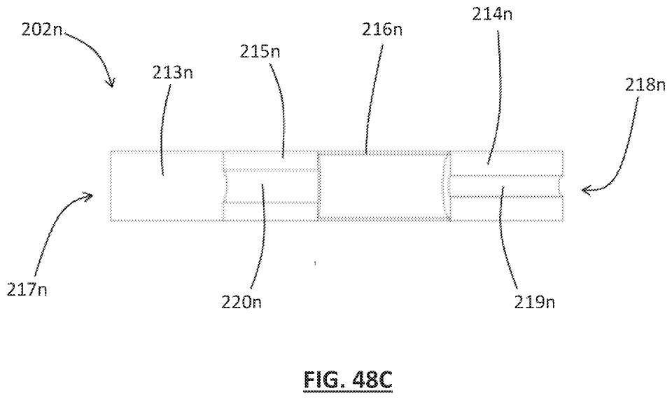

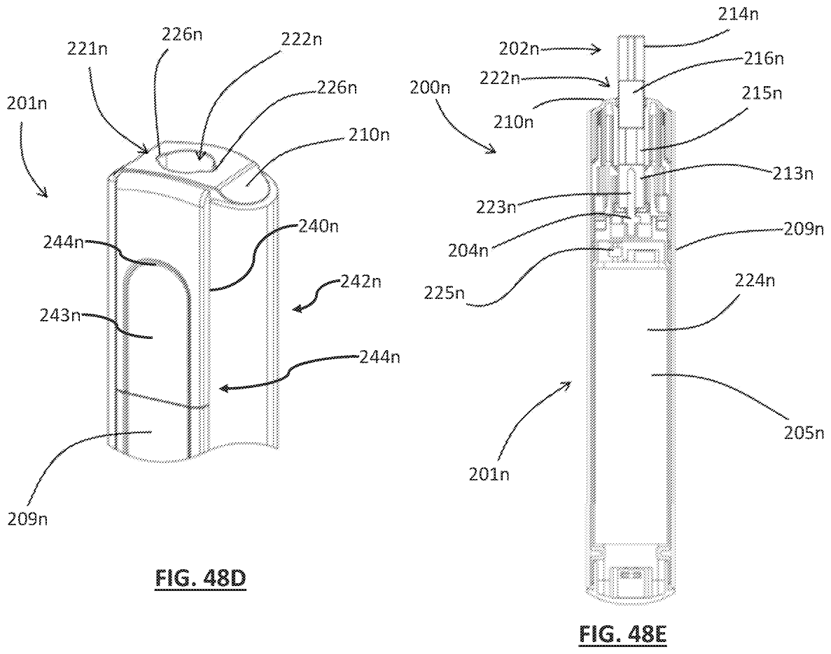

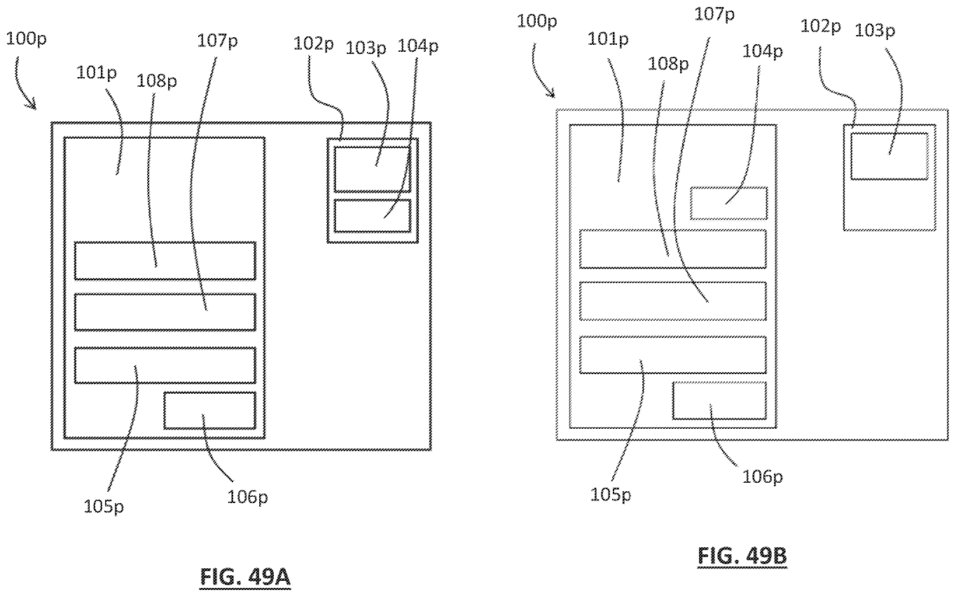

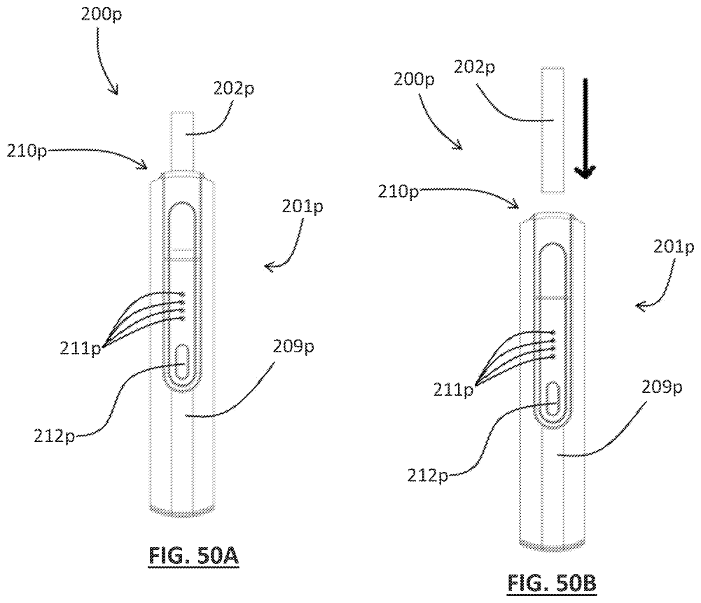

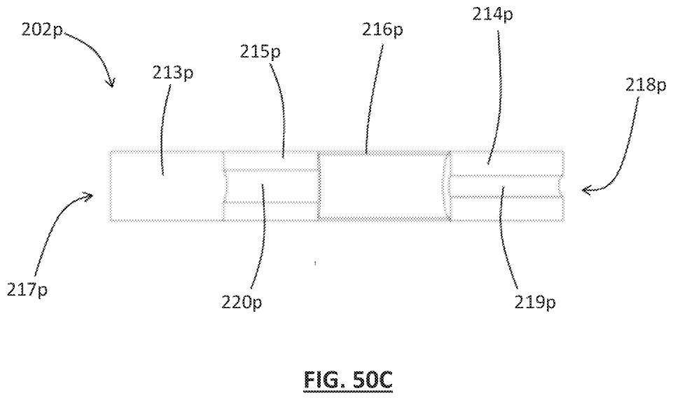

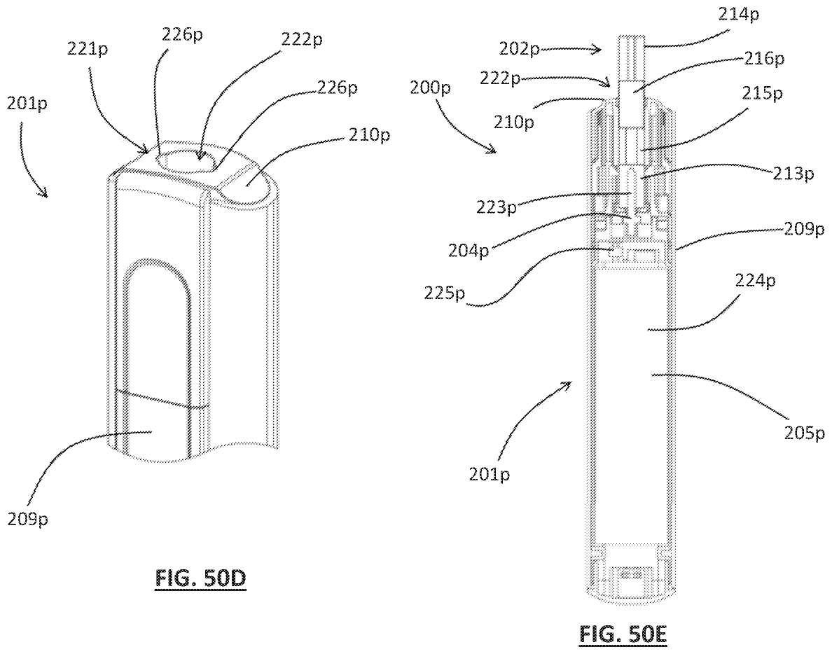

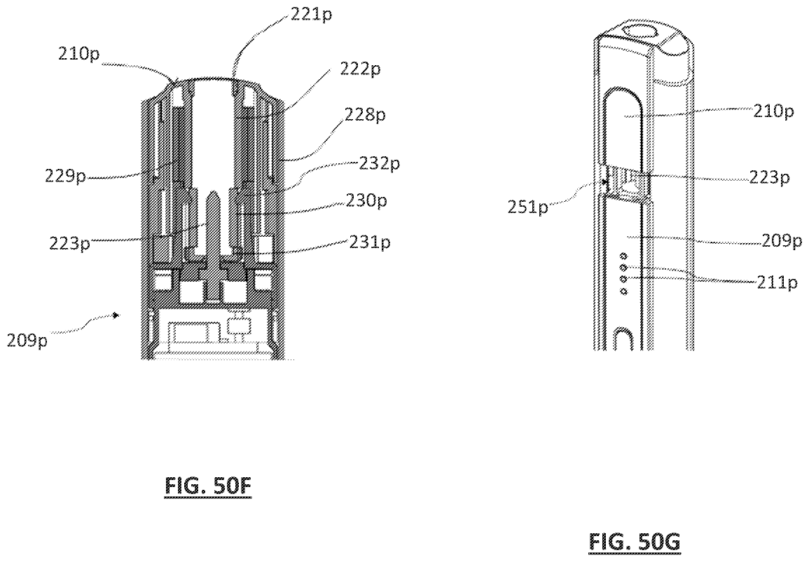

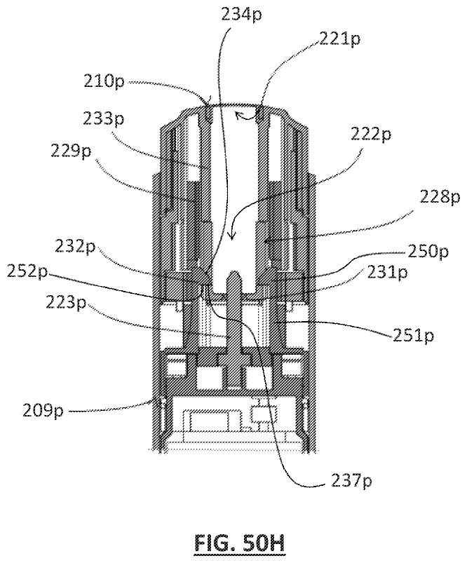

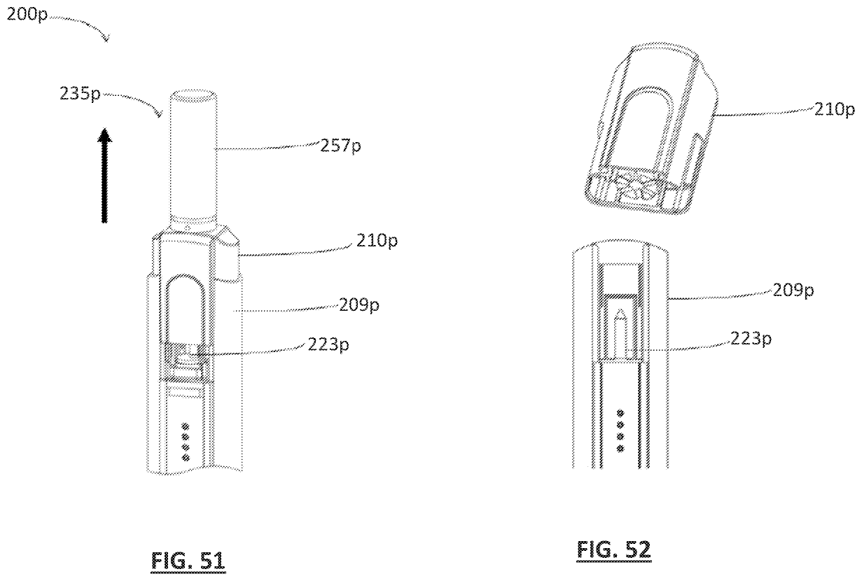

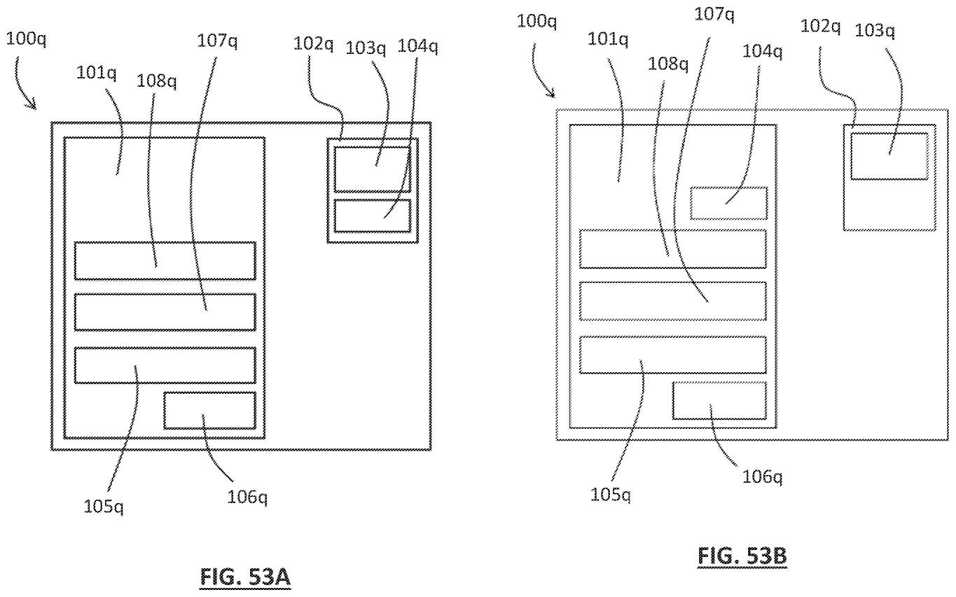

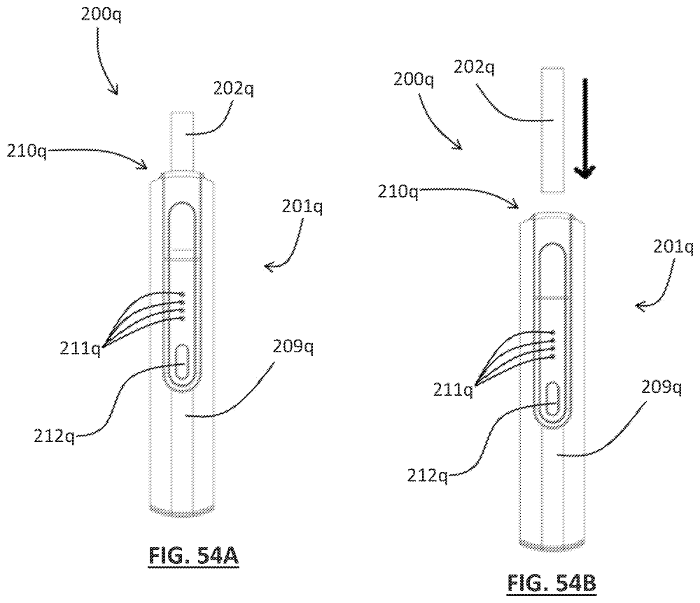

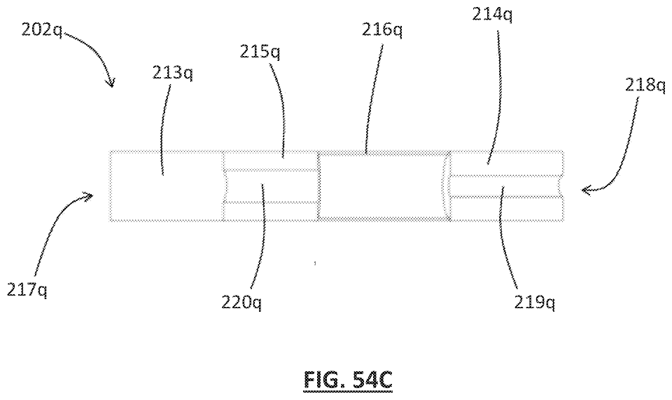

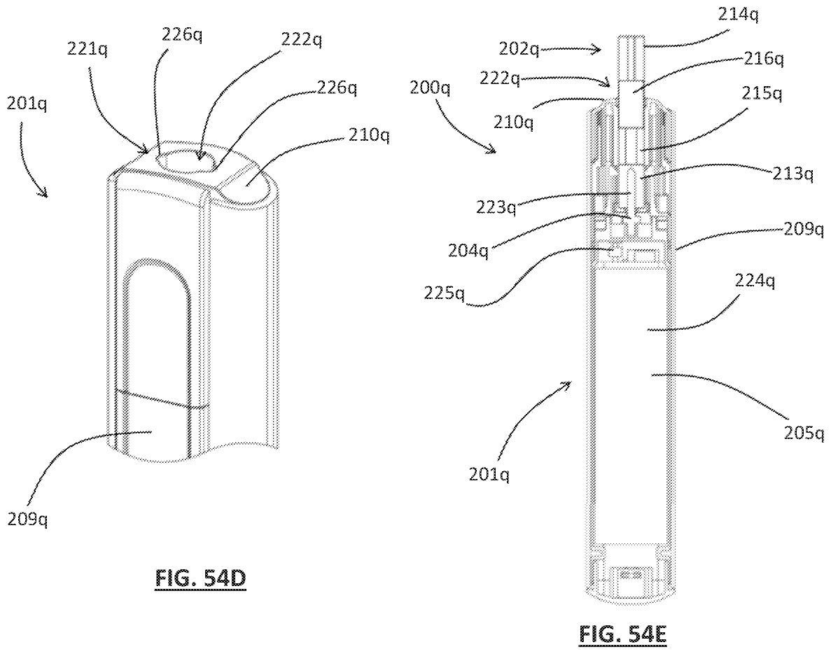

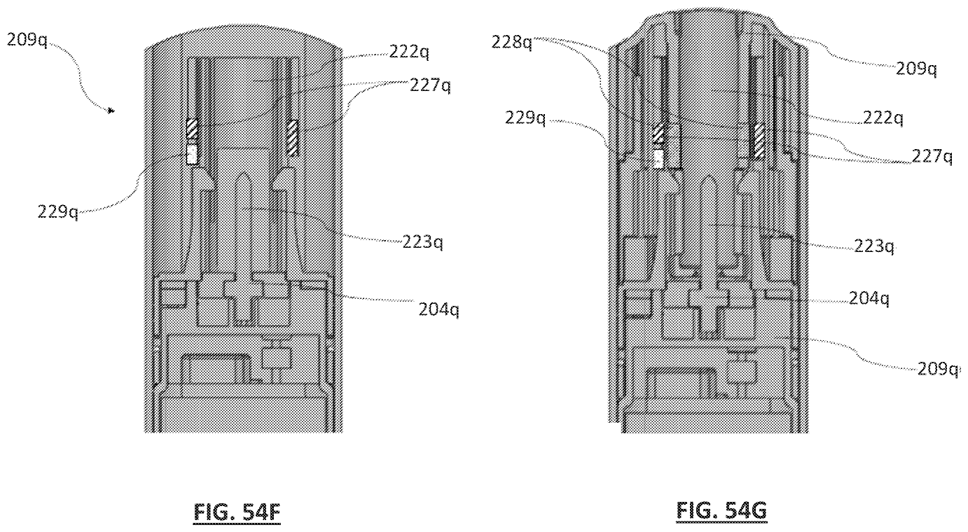

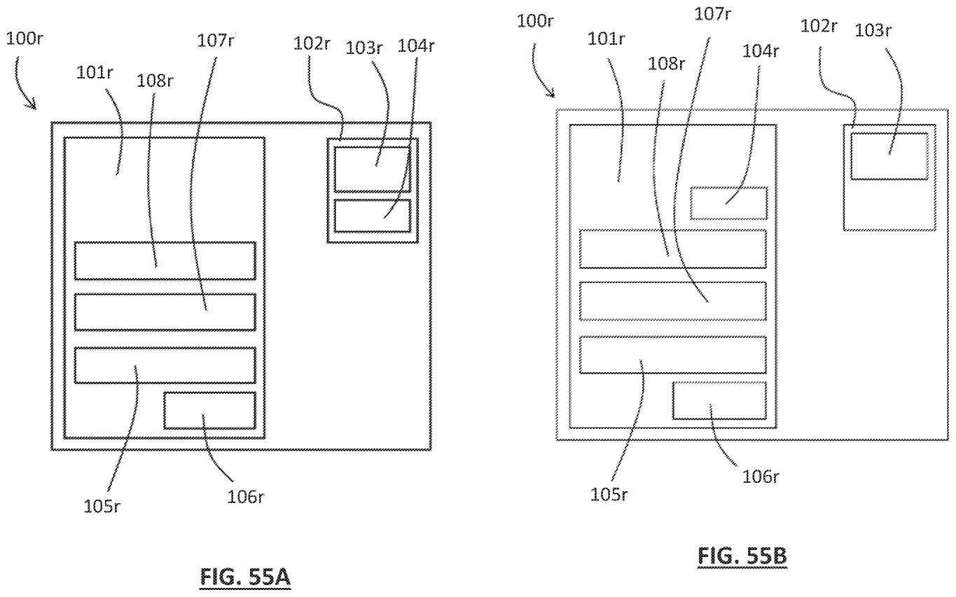

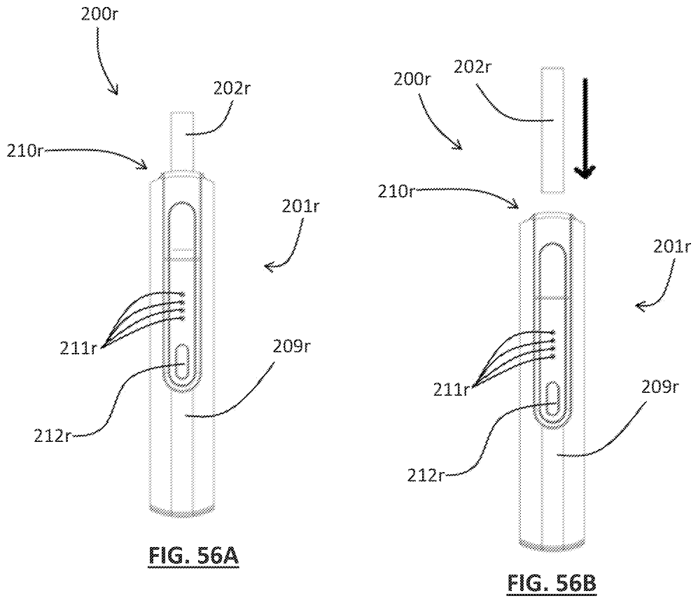

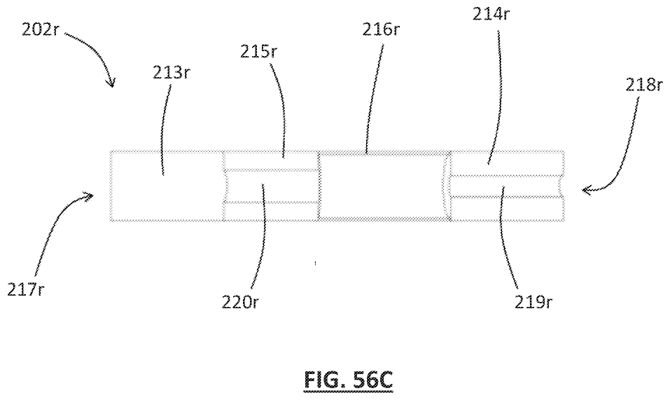

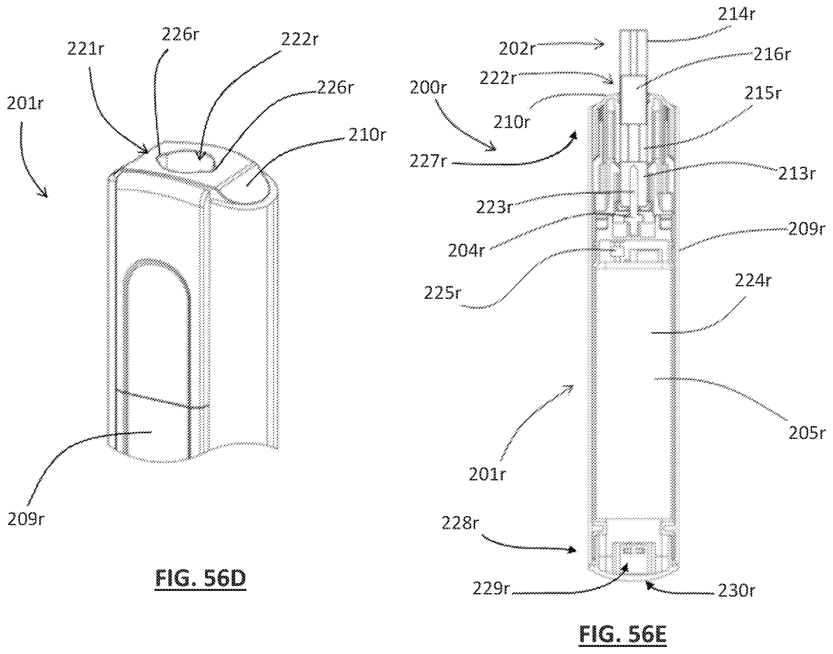

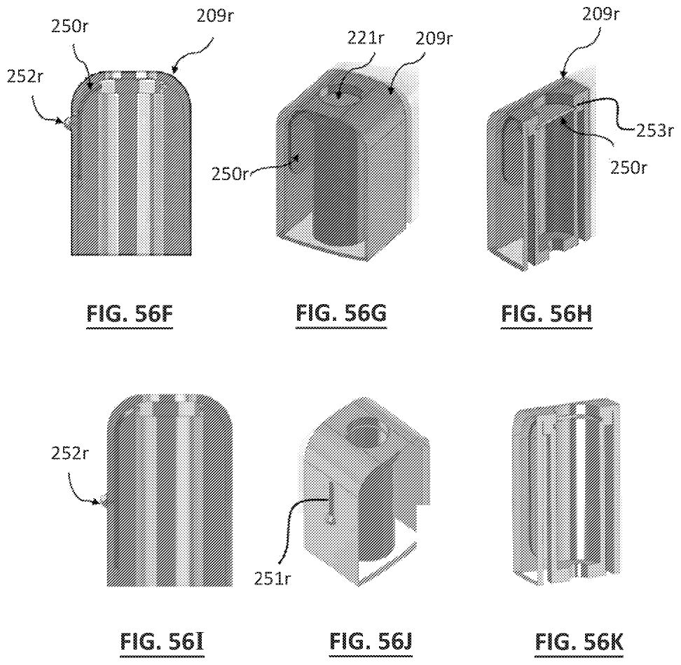

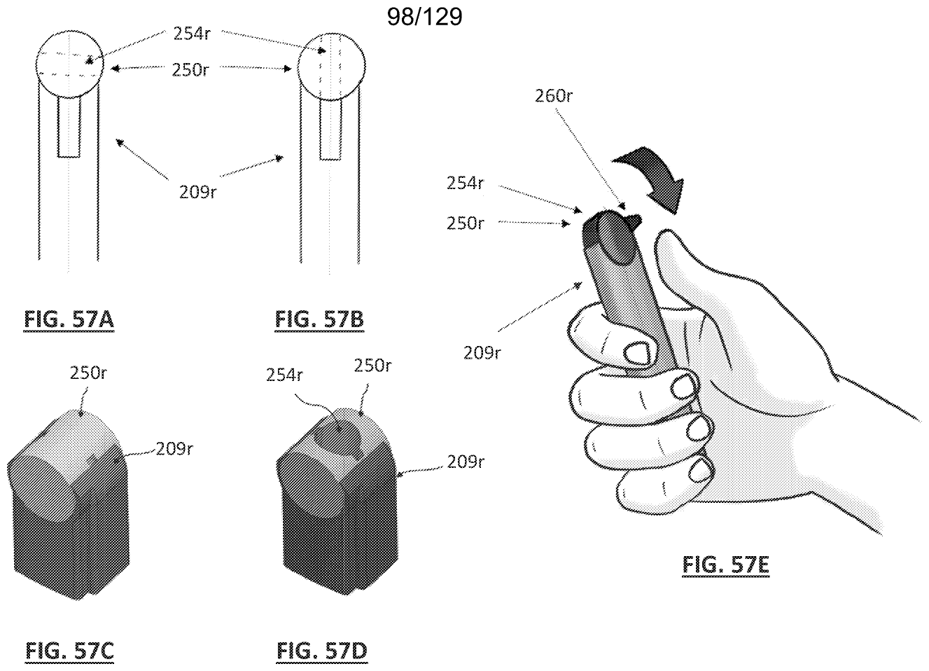

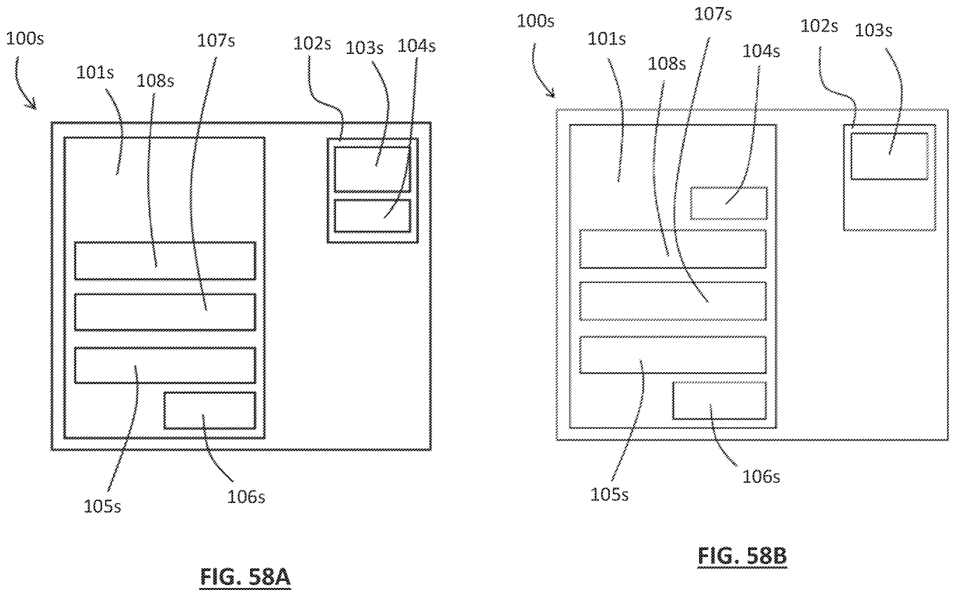

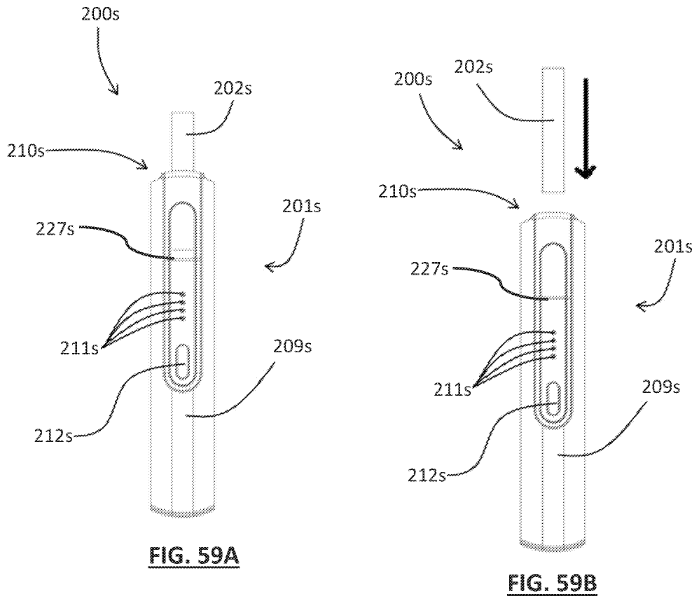

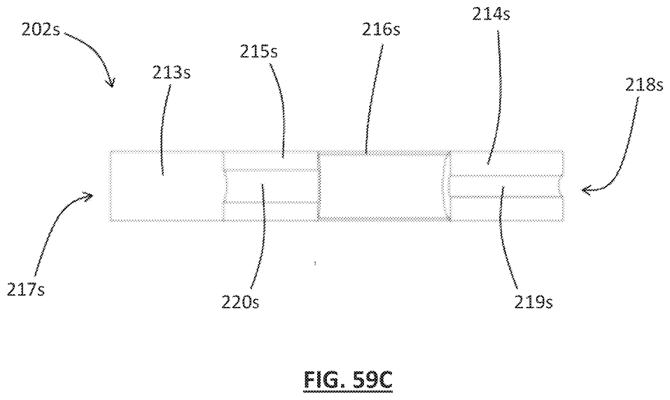

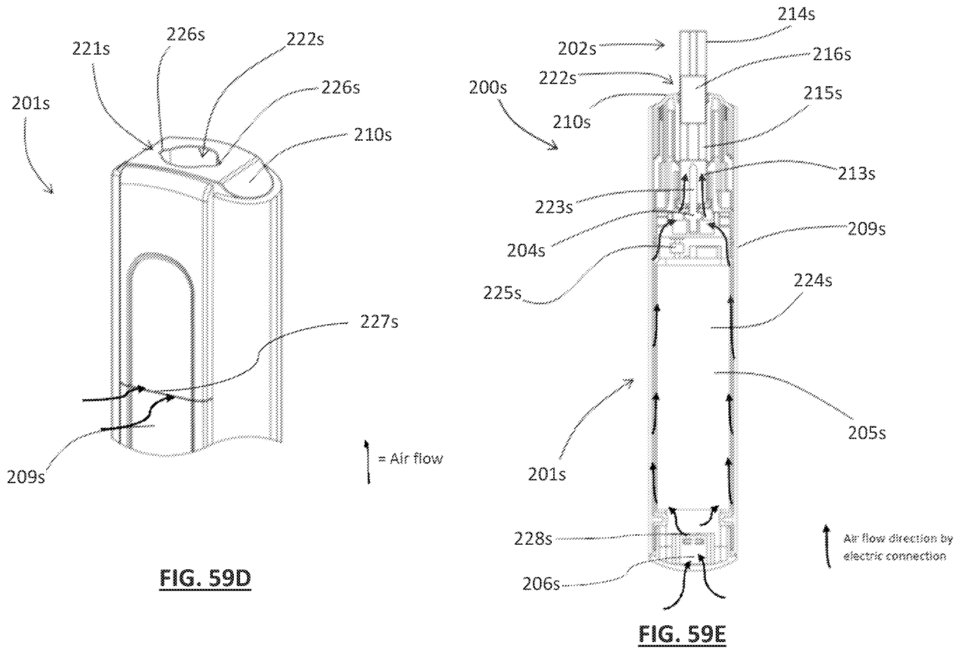

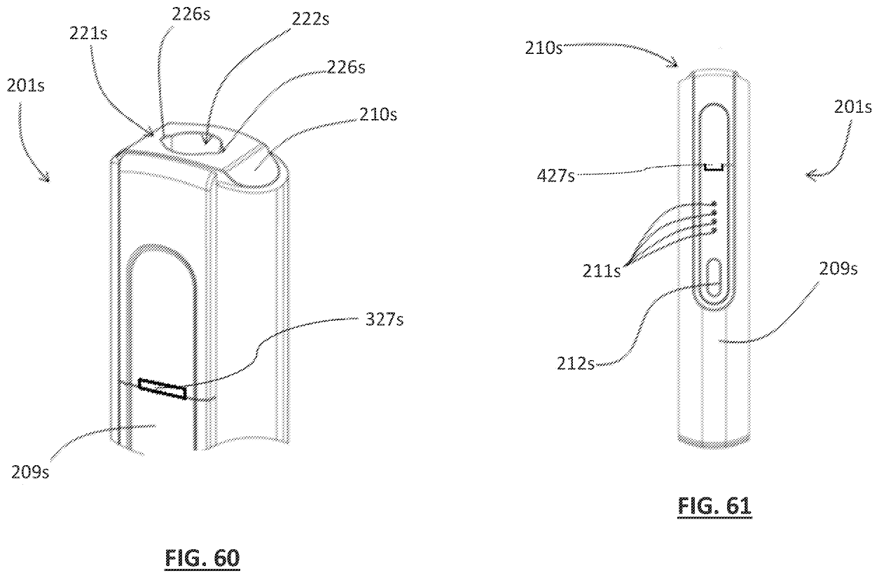

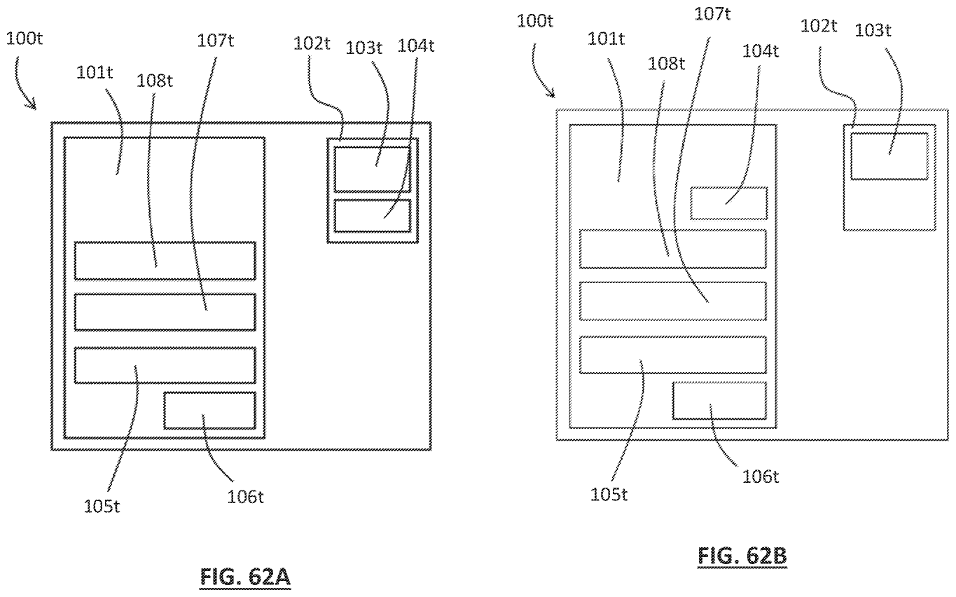

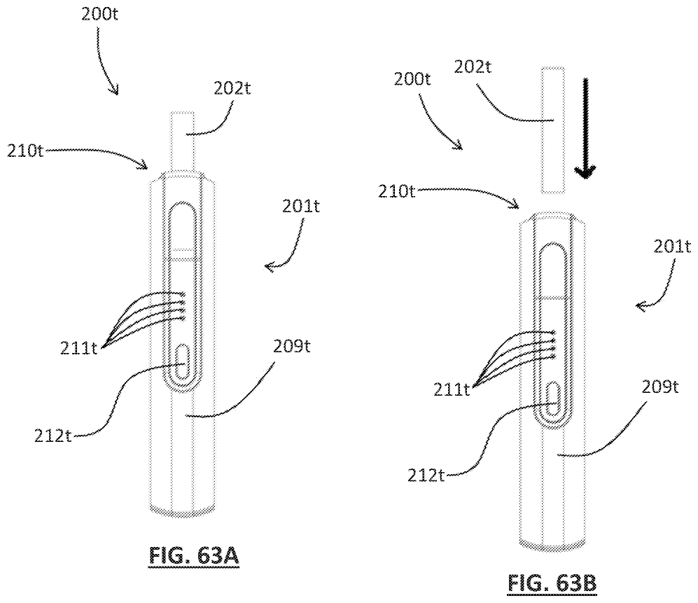

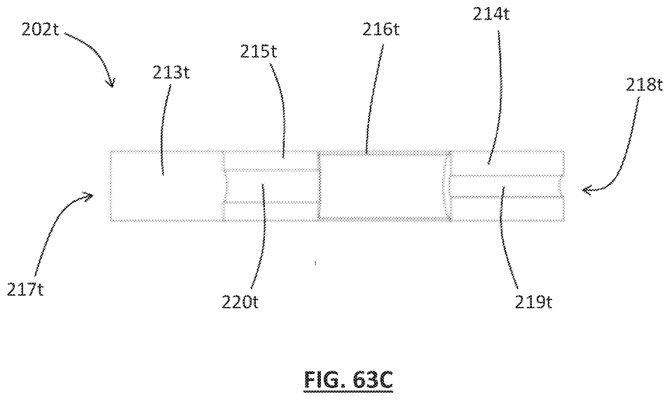

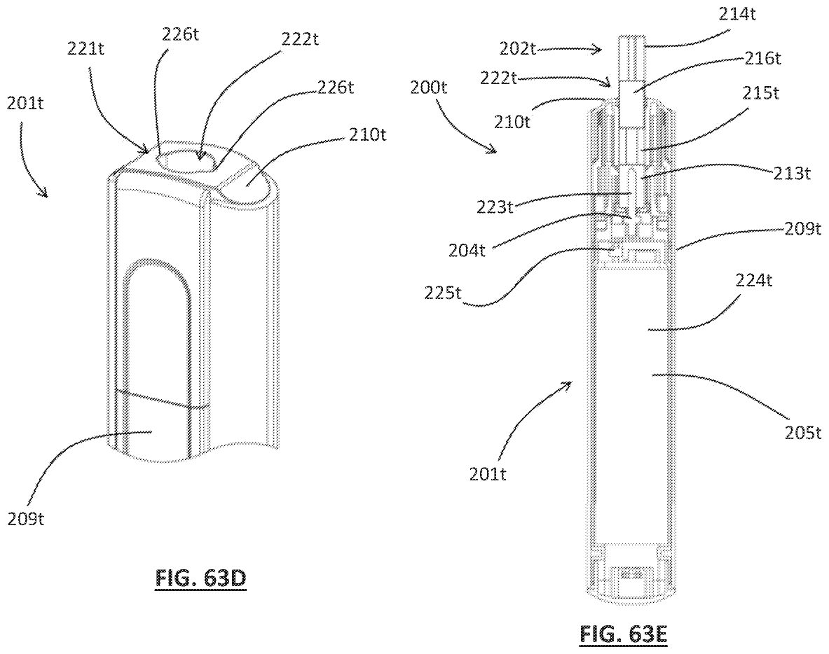

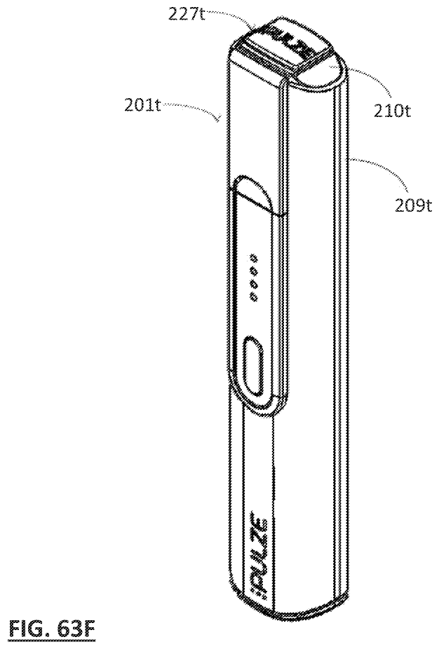

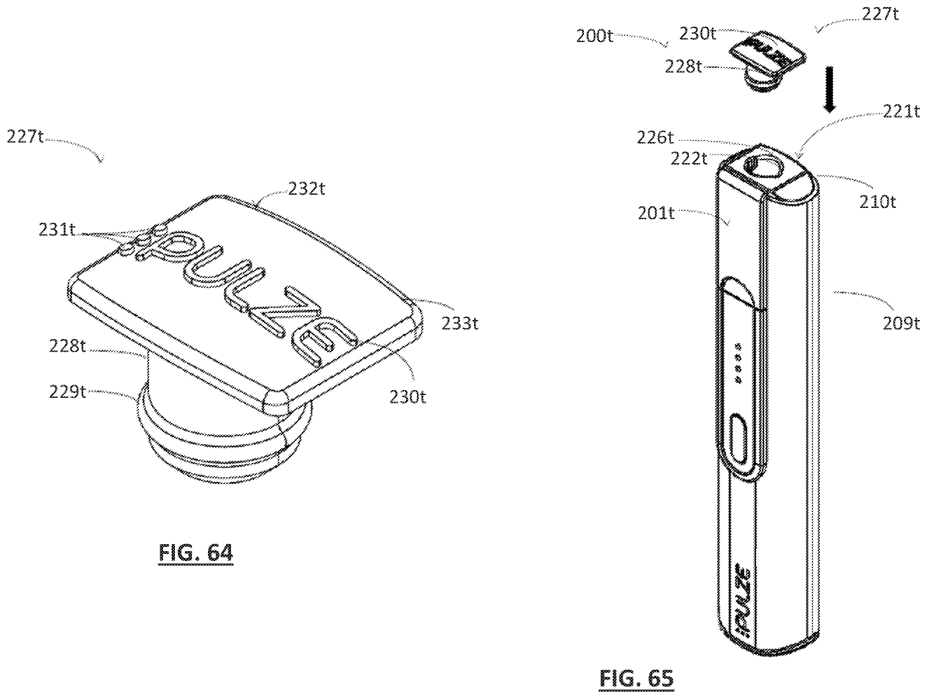

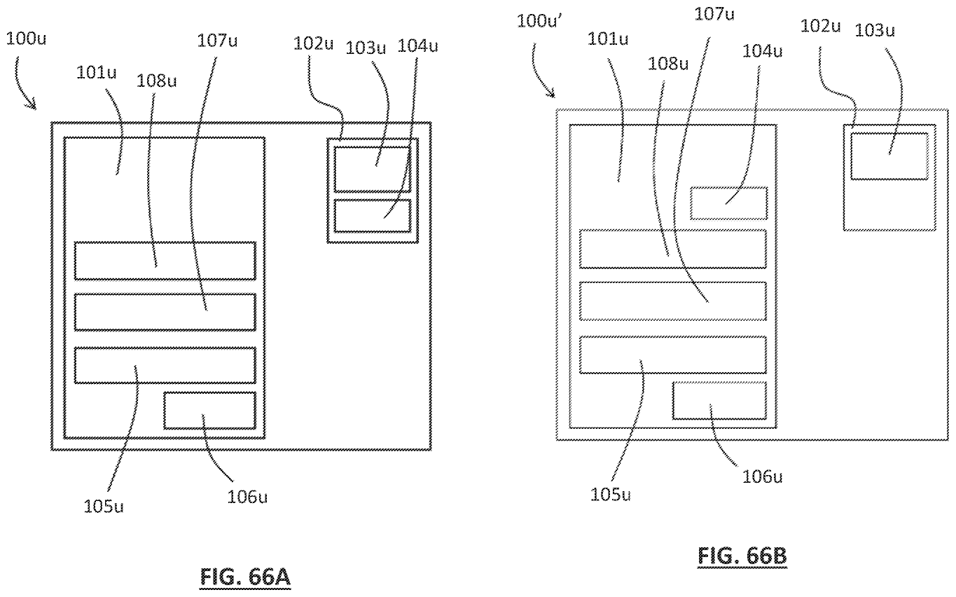

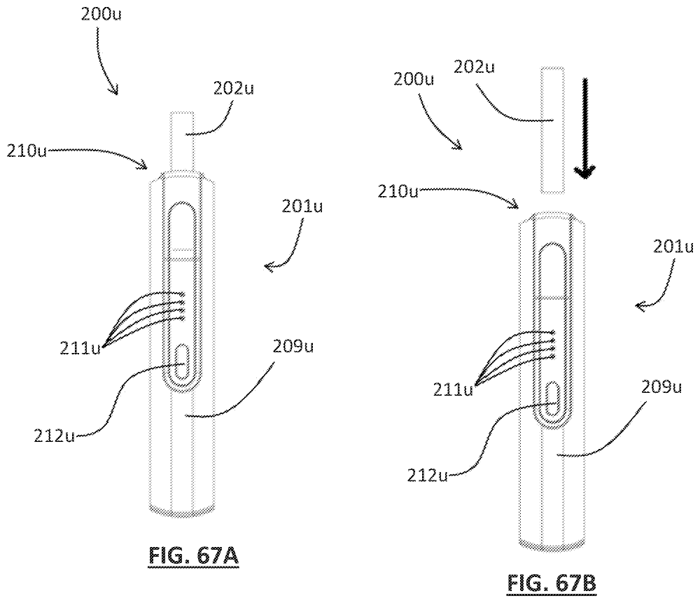

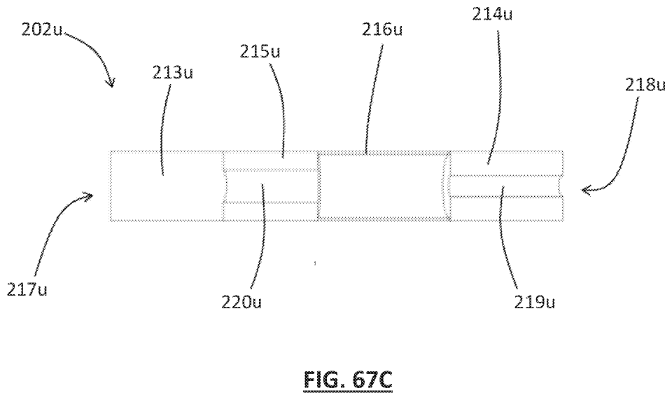

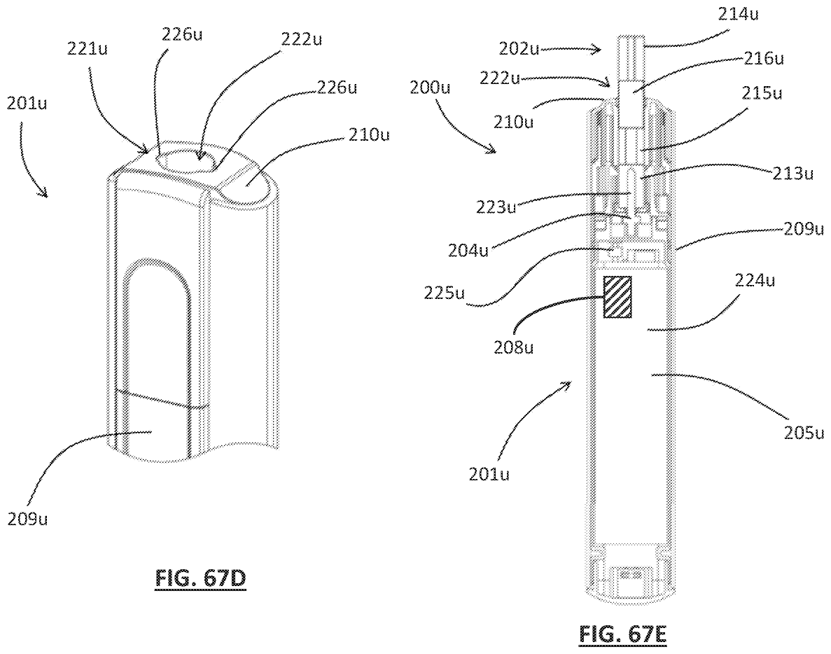

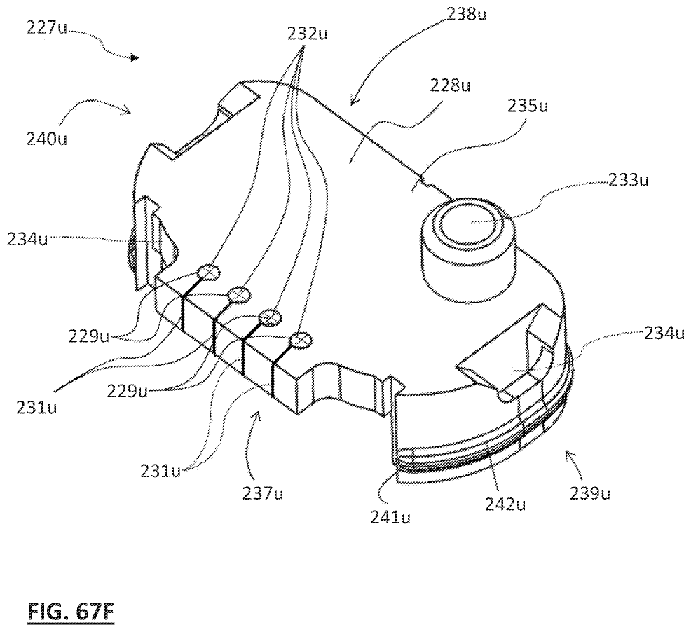

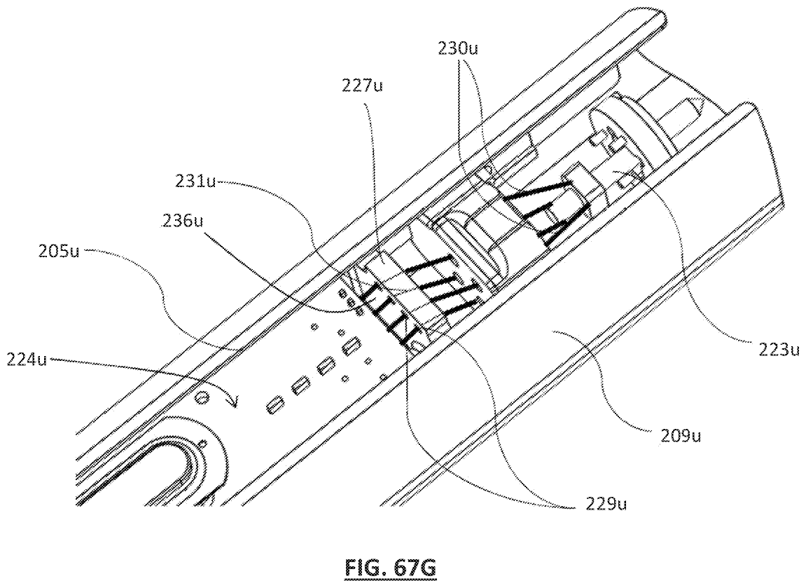

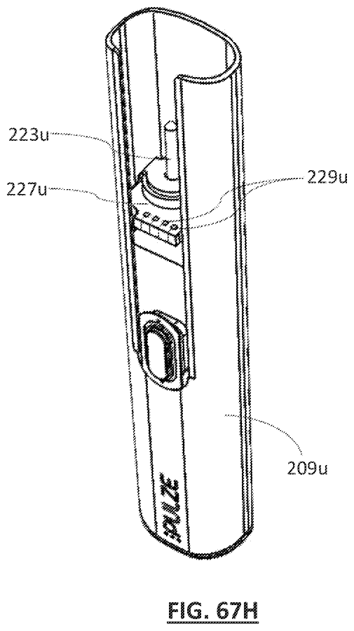

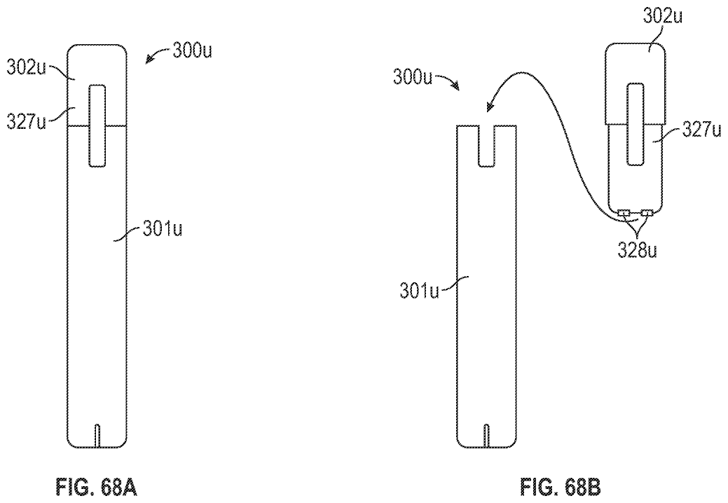

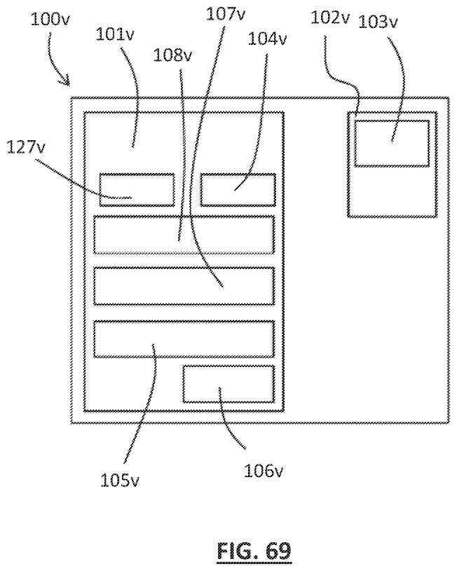

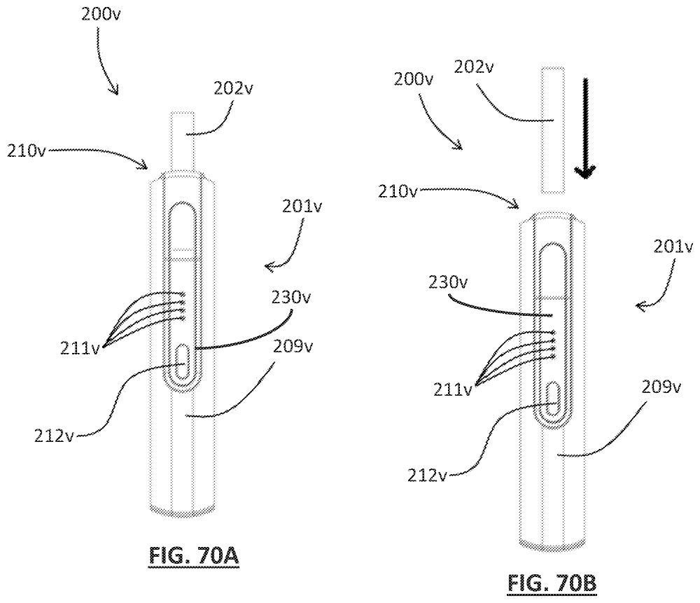

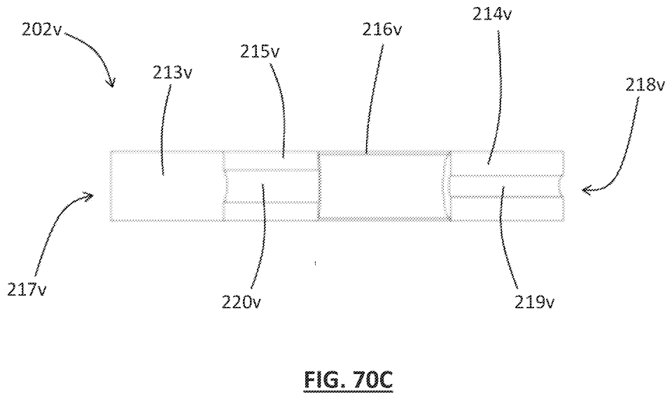

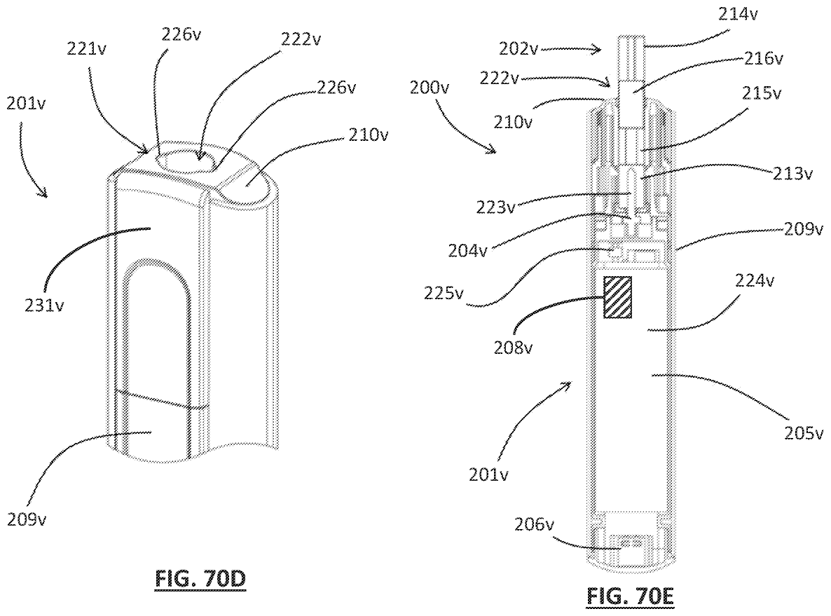

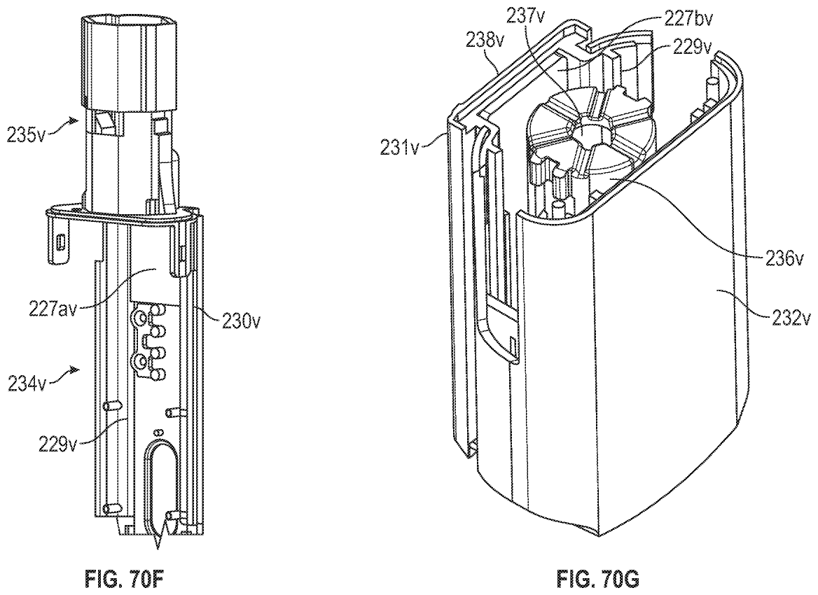

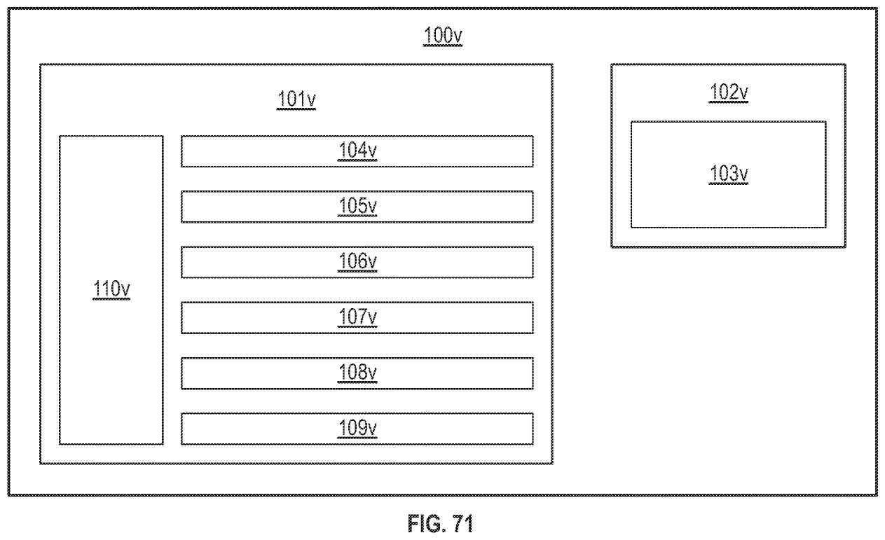

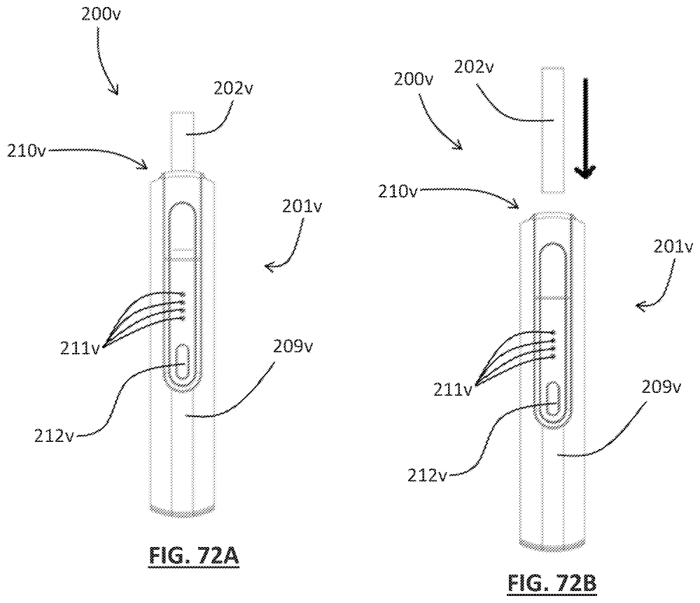

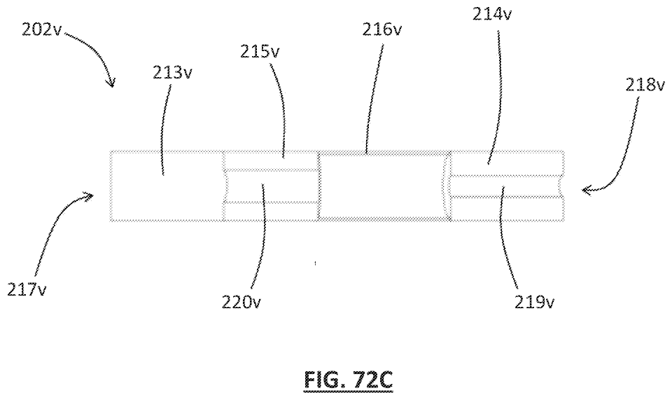

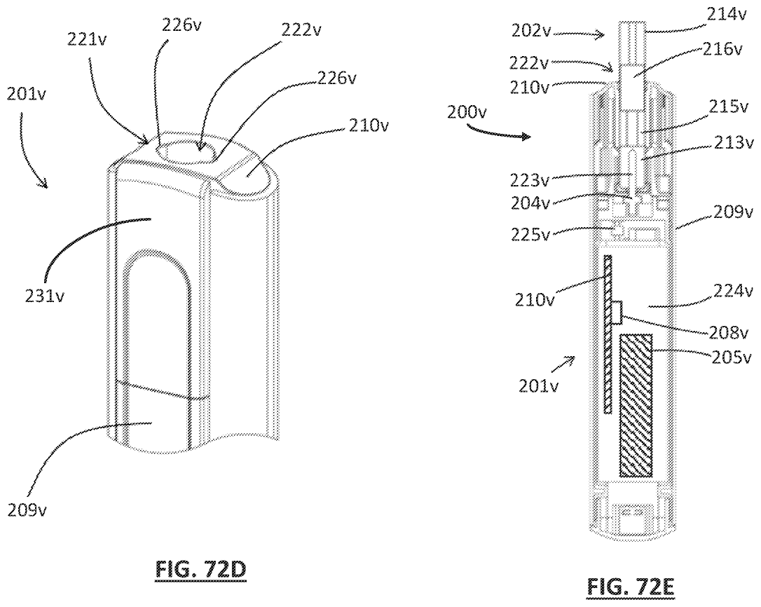

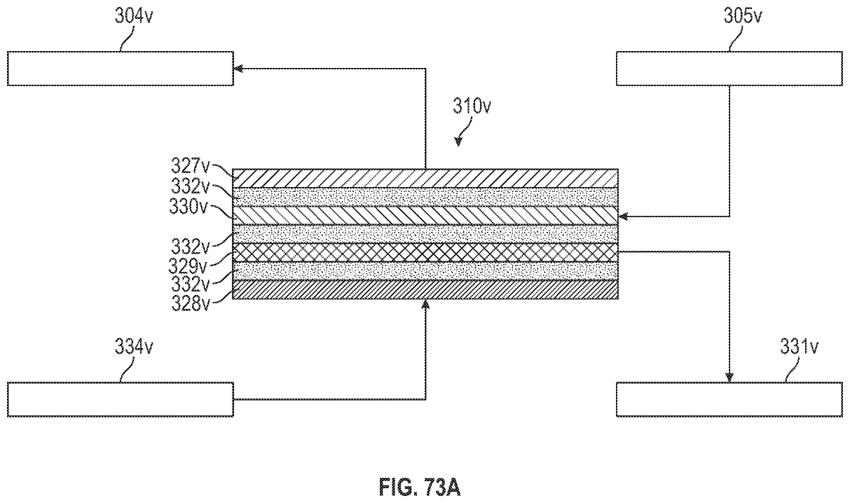

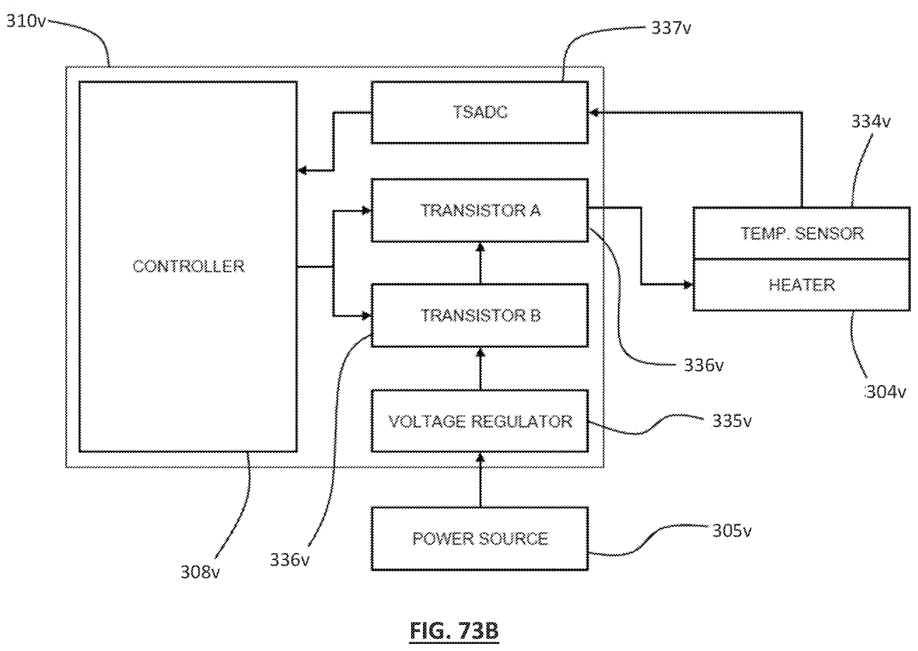

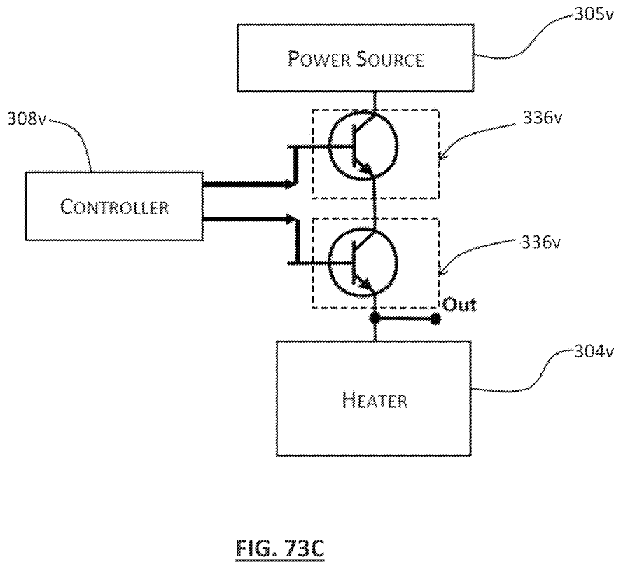

FIG. 10 illustrates a tool 250b inserted into the cap 210b for removing the cap 210b from the body of the device. The tool 250b is configured to interact with the retainer mechanism (i.e., the flexure bearing 232b). The flexure bearings 232b are deformed to move outwardly away from the deformable region 230b and the heating element 223b of the device when the tool is engaged. This outward movement of the flexure bearing 232b facilitates the movement of the cap 210b beyond the second position and its disengagement from the body 209b (as seen in FIG. 11).

### CONCLUSION

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilized for realizing the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

1.-53. (canceled)

54. A smoking substitute device comprising: a body;
a heating element extending from the body;
a cap removably attached to the body, the cap being movable between a first position and a second position along a longitudinal axis of the body, wherein in the first position the heating element is concealed in the cap and in the second position the heating element is at least partially exposed.

55. The smoking substitute device of claim 54, wherein
the cap is retainable on the body in the second position by a retaining means.

56. The smoking substitute device of claim 55, wherein
the retaining means comprises:
at least one flexible locking arm extending from the body; and
a locking protrusion disposed on the at least one locking arm, the locking protrusion is configured to engage a slot defined in the cap to retain the cap on the body.

57. The smoking substitute device of claim 56, wherein the locking protrusion is retained in the slot when the cap moves between the first position and the second position.

58. The smoking substitute device of claim 54, wherein the body defines a transverse cavity that opens through a first side wall of the body, the transverse cavity juxtaposed with a base of the heating element to at least partially expose the base of the heating element when the cap is in the second position.

59. The smoking substitute device of claim 58, wherein the transverse cavity extends from the first side wall of the body to and through a second side wall opposite to the first side wall.

60. The smoking substitute device of claim 56, wherein the locking protrusion is configured to prevent separation of the cap from the device by abutting an end of the slot once the cap has moved to the second position, as such blocking further movement of the cap.

61. The smoking substitute device of claim 60, wherein the cap defines a cavity for receiving at least a portion of an aerosol-forming article.

62. A smoking substitute system, comprising:
the smoking substitute device of claim 54; and
an aerosol-forming article for the smoking substitute device.

63.-178. (canceled)

179. A smoking substitute device comprising:
a body;
a heating element projecting from the body along a longitudinal axis; and
a cap engageable with the body for at least partly enclosing the heating element, the cap moveable away from the body along the longitudinal axis, whilst remaining engaged with the body, so as to define an aperture for accessing the heating element.

180. The smoking substitute device according to claim 179, wherein the cap defines a cavity to receive a consumable.

181. The smoking substitute device according to claim 179, wherein the heating element is configured to penetrate into at least a portion of the consumable.

182. The smoking substitute device according to claim 179, wherein the cap is movable between a first position and a second position relative to the body, along the longitudinal axis, wherein the first position corresponds to a fully engaged condition of the cap with the body, and the second position corresponds to a lifted condition of the cap with respect to the body, defining the aperture.

183. The smoking substitute device according to claim 182, wherein movement of the cap from the first position to the second position facilitates lifting of at least a portion of the consumable away from the heating element along the longitudinal axis.

184. The smoking substitute device according to claim 182, wherein the displacement of the cap between the first position and the second position ranges from about 2 mm to about 15 mm, preferably from about 5 mm to about 8 mm.

185. The smoking substitute device according to claim 182, further comprising a retainer mechanism to retain the cap in one or more of the first position and the second position.

186. The smoking substitute device according to claim 185, wherein the retainer mechanism is configured to restrict movement of the cap beyond the second position.

187. The smoking substitute device according to claim 185 wherein the retainer mechanism is at least one of a detent mechanism and a magnetic lock mechanism.

188. The smoking substitute device according to claim 179, wherein the cap is engageable with the body by a sliding mechanism or a threading mechanism.

189. The smoking substitute device according to claim 179, wherein the cap is removable from the body when in the second position by means of a tool.

190. The smoking substitute device according to claim 179, wherein at least a portion of the cap is configured with a tactile finish to facilitate gripping of the cap for movement of the cap between the first position and the second position.

191. A smoking substitute system, comprising:
a device according to claim 179; and an aerosol-forming article.

192. The smoking substitute system according to claim 191, wherein the article is a heat-not-burn (HNB) consumable.

193.-295. (canceled)
